(19) United States
(12) Patent Application Publication
(10) Pub. No.: US 2022/0267415 A1
(43) Pub. Date: Aug. 25, 2022

(54) MULTIMERIC SARS-COV-2 BINDING MOLECULES AND USES THEREOF

(71) Applicants: The Board of Regents of The University of Texas System, Austin, TX (US); IGM Biosciences, Inc., Mountain View, CA (US)

(72) Inventors: Zhiqiang KU, Houston, TX (US); Xuping XIE, Galveston, TX (US); Paul HINTON, Sunnyvale, CA (US); Ningyan ZHANG, Houston, TX (US); Bruce KEYT, Hillsborough, CA (US); Dean NG, San Francisco, CA (US); Stephen CARROLL, Walnut Creek, CA (US); Pei-Yong SHI, Galveston, TX (US); Zhiqiang AN, Houston, TX (US)

(73) Assignees: The Board of Regents of The University of Texas System, Austin, TX (US); IGM Biosciences, Inc., Mountain View, CA (US)

(21) Appl. No.: 17/671,837

(22) Filed: Feb. 15, 2022

Related U.S. Application Data
(60) Provisional application No. 63/150,570, filed on Feb. 17, 2021.
(60) Provisional application No. 63/151,624, filed on Feb. 19, 2021.
(60) Provisional application No. 63/178,604, filed on Apr. 23, 2021.
(60) Provisional application No. 63/194,572, filed on May 28, 2021.
(60) Provisional application No. 63/291,004, filed on Dec. 17, 2021.

(51) Int. Cl. C07K 16/10; A61P 31/14

(52) U.S. Cl. CPC C07K 16/10; A61P 31/14; A61K 2039/505; C07K 2317/35; C07K 2317/31

(57) ABSTRACT

This disclosure provides multimeric binding molecules that bind to SARS-CoV-2. This disclosure also provides compositions comprising the multimeric binding molecules, polynucleotides that encode the multimeric binding molecules, and host cells that can produce the binding molecules. Further this disclosure provides methods of using the multimeric binding molecules, including methods for treating and preventing coronavirus disease 2019 (COVID-19).

### CROSS-REFERENCE TO RELATED APPLICATIONS

This application claims the benefit of U.S. Provisional Patent Application Ser. No. 63/150,570, filed Feb. 17, 2021, U.S. Provisional Patent Application Ser. No. 63/151,624, filed Feb. 19, 2021, U.S. Provisional Patent Application Ser. No. 63/178,604, filed Apr. 23, 2021, U.S. Provisional Patent Application Ser. No. 63/194,572, filed May 28, 2021, and U.S. Provisional Patent Application Ser. No. 63/291,004, filed Dec. 17, 2021, each of which is incorporated herein by reference in its entirety.

### REFERENCE TO A SEQUENCE LISTING

This application contains references to amino acid sequences and/or nucleic acid sequences which have been submitted concurrently herewith as the sequence listing text file “UTSHP0373US_updated_ST25.txt”, file size 80,031 bytes, created on 28 Mar. 2022. The aforementioned sequence listing is hereby incorporated by reference in its entirety pursuant to 37 C.F.R. § 1.52(e)(5).

### BACKGROUND

Antibodies and antibody-like molecules that can multimerize, such as IgA and IgM antibodies, have emerged as promising drug candidates, e.g., in the fields of immuno-oncology and infectious diseases, allowing for improved specificity, improved avidity, and the ability to bind to multiple binding targets. See, e.g., U.S. Pat. Nos. 9,951,134, 9,938,347, 10,351,631, 10,400,038, and 10,899,835, U.S. Patent Application Publication Nos. US 2019-0100597, US 2018-0009897, US 2019-0330374, US 2019-0330360, US 2019-0338040, US 2019-0338041, US 2019-0185570, US 2018-0265596, US 2018-0118816, US 2018-0118814, and US 2019-0002566, and PCT Publication Nos. WO 2018/187702 and WO 2019/165340, the contents of which are incorporated herein by reference in their entireties.

Severe acute respiratory syndrome coronavirus 2 (SARS-CoV-2) is a single stranded, positive sense enveloped RNA virus. SARS-CoV-2 causes Coronavirus Disease 2019 (COVID-19), which is currently causing a global pandemic. COVID-19 is highly contagious and commonly causes fever, cough, and shortness of breath, and can lead to pneumonia, blood clots, organ failure, and death. The four main structural proteins of the SARS-CoV-2 include spike (S), envelope (E), membrane (M), and nucleic capsid (N).

The trimeric S protein binds the angiotensin-converting enzyme 2 (ACE2) receptor, alters its conformation to a fusogenic protein, which facilitates fusion of the cellular and viral membranes and thereby enables SARS-CoV-2 to enter cells. The S protein comprises two units: 51 and S2, with the 51 domain comprising the receptor-binding domain (RBD). See, e.g., Lan, J., et al., Nature 581:215-220 (2020). The S protein and specifically the RBD are required for entry into cells, which has made the RBD a favored target of potential therapeutic monoclonal antibodies. Unfortunately, even the IgG antibodies having very potent neutralizing activity against the RBD of SARS-CoV-2 need to be administered by infusion at high doses (up to 8 grams per dose) to effectively treat COVID-19 patients (Weinreich, D M, et al., N Engl J Med doi: 10.1056/NEJMoa2035002 I2020 (2021); Chen, et al., N Engl J Med doi: 10.1056/NEJMoa2029849 (2021)).

There remains an urgent need for therapeutics to treat and/or prevent COVID-19.

### SUMMARY

This application provides a multimeric binding molecule that includes two to six bivalent binding units or variants or fragments thereof, where each binding unit includes two IgM or IgA heavy chain constant regions or multimerizing fragments or variants thereof, each associated with a binding domain, where three to twelve of the binding domains are identical immunoglobulin antigen binding domains that specifically bind to the SARS-CoV-2 spike (S) protein receptor binding domain (RBD), where each identical immunoglobulin antigen binding domain includes a heavy chain variable region (VH) and a light chain variable region (VL) that includes six immunoglobulin complementarity determining regions HCDR1, HCDR2, HCDR3, LCDR1, LCDR2, and LCDR3, where the HCDR1, HCDR2, HCDR3, LCDR1, LCDR2, and LCDR3, include, respectively, the amino acid sequences SEQ ID NO: 15, SEQ ID NO: 16, SEQ ID NO: 17, SEQ ID NO 19, SEQ ID NO. 20, and SEQ ID NO: 21; SEQ ID NO: 23, SEQ ID NO: 24, SEQ ID NO: 25, SEQ ID NO 27, SEQ ID NO. 28, and SEQ ID NO: 29; SEQ ID NO: 31, SEQ ID NO: 32, SEQ ID NO: 33, SEQ ID NO 35, SEQ ID NO. 36, and SEQ ID NO: 37; SEQ ID NO: 39, SEQ ID NO: 40, SEQ ID NO: 41, SEQ ID NO 43, SEQ ID NO. 44, and SEQ ID NO: 45; SEQ ID NO: 47, SEQ ID NO: 48, SEQ ID NO: 49, SEQ ID NO 51, SEQ ID NO. 52, and SEQ ID NO: 53; or SEQ ID NO: 55, SEQ ID NO: 56, SEQ ID NO: 57, SEQ ID NO 59, SEQ ID NO. 60, and SEQ ID NO: 61; where the CDR regions are defined according to Kabat; or where each identical immunoglobulin antigen binding domain includes a heavy chain variable region (VH) and a light chain variable region (VL) that includes six immunoglobulin complementarity determining regions HCDR1, HCDR2, HCDR3, LCDR1, LCDR2, and LCDR3, where the HCDR1, HCDR2, HCDR3, LCDR1, LCDR2, and LCDR3, include, respectively, the amino acid sequences SEQ ID NO: 62, SEQ ID NO: 63, SEQ ID NO: 64, SEQ ID NO: 65, SEQ ID NO: 66, and SEQ ID NO: 67; SEQ ID NO: 68, SEQ ID NO: 69, SEQ ID NO: 70, SEQ ID NO: 71, SEQ ID NO: 72, and SEQ ID NO: 73; SEQ ID NO: 74, SEQ ID NO: 75, SEQ ID NO: 76, SEQ ID NO: 77, SEQ ID NO: 78, and SEQ ID NO: 79; SEQ ID NO: 80, SEQ ID NO: 81, SEQ ID NO: 82, SEQ ID NO: 83, SEQ ID NO: 84, and SEQ ID NO: 85; SEQ ID NO: 86, SEQ ID NO: 87, SEQ ID NO: 88, SEQ ID NO: 89, SEQ ID NO: 90, and SEQ ID NO: 91; or SEQ ID NO: 92, SEQ ID NO: 93, SEQ ID NO: 94, SEQ ID NO: 95, SEQ ID NO: 96, and SEQ ID NO: 97; where the CDR regions are defined according to IMGT. In certain embodiments the provided multimeric binding molecule has greater antiviral potency against SARS-CoV-2 than a bivalent reference IgG antibody that includes two of the binding domains that specifically bind to the SARS-CoV-2 S protein RBD. In certain embodiments the bivalent reference IgG antibody includes two identical antigen binding domains each including the VH and VL amino acid sequences SEQ ID NO: 14 and SEQ ID NO: 18, SEQ ID NO: 22 and SEQ ID NO: 26, SEQ ID NO: 30 and SEQ ID NO: 34, SEQ ID NO: 38 and SEQ ID NO: 42, SEQ ID NO: 46 and SEQ ID NO: 50, or SEQ ID NO: 54 and SEQ ID NO: 58, respectively.

In certain embodiments each VH and VL of the provided multimeric binding molecule include the amino acid sequences SEQ ID NO: 14 and SEQ ID NO: 18, SEQ ID NO: 22 and SEQ ID NO: 26, SEQ ID NO: 30 and SEQ ID NO: 34, SEQ ID NO: 38 and SEQ ID NO: 42, SEQ ID NO: 46 and SEQ ID NO: 50, or SEQ ID NO: 54 and SEQ ID NO: 58, respectively.

In certain embodiments each HCDR1, HCDR2, HCDR3, LCDR1, LCDR2, and LCDR3 of the provided multimeric binding molecule include, respectively, the amino acid sequences SEQ ID NO: 15, SEQ ID NO: 16, SEQ ID NO: 17, SEQ ID NO 19, SEQ ID NO. 20, and SEQ ID NO: 21, or SEQ ID NO: 39, SEQ ID NO: 40, SEQ ID NO: 41, SEQ ID NO 43, SEQ ID NO. 44, and SEQ ID NO: 45; where the CDR regions are defined according to Kabat, or the amino acid sequences SEQ ID NO: 62, SEQ ID NO: 63, SEQ ID NO: 64, SEQ ID NO: 65, SEQ ID NO: 66, and SEQ ID NO: 67; or SEQ ID NO: 80, SEQ ID NO: 81, SEQ ID NO: 82, SEQ ID NO: 83, SEQ ID NO: 84, and SEQ ID NO: 85; where the CDR regions are defined according to IMGT. In certain embodiments each VH and VL regions of the provided multimeric binding molecule include the amino acid sequences SEQ ID NO: 14 and SEQ ID NO: 18 or SEQ ID NO: 38 and SEQ ID NO: 42, respectively.

In certain embodiments each VH and VL of the provided multimeric binding molecule include the amino acid sequences SEQ ID NO: 14 and SEQ ID NO: 18, respectively, and each VH and VL of the bivalent reference IgG antibody include the amino acid sequences SEQ ID NO: 14 and SEQ ID NO: 18, respectively. In certain embodiments each VH and VL of the multimeric binding molecule include the amino acid sequences SEQ ID NO: 38 and SEQ ID NO: 42, respectively, and each VH and VL of the bivalent reference IgG antibody include the amino acid sequences SEQ ID NO: 38 and SEQ ID NO: 42, respectively.

In certain embodiments the greater antiviral potency of the provided multimeric binding molecule against SARS-CoV-2 includes a) inhibition of binding of the SARS-CoV-2 spike protein to its receptor angiotensin-converting enzyme 2 (ACE2) at a lower 50% effective concentration (EC₅₀) than the bivalent reference IgG antibody, b) inhibition of binding of the SARS-CoV-2 spike protein to ACE2 under conditions where the bivalent reference IgG antibody cannot inhibit binding, c) neutralization of SARS-CoV-2 infectivity at a lower EC₅₀ than the bivalent reference IgG antibody, d) neutralization of SARS-CoV-2 infectivity under conditions where the bivalent reference IgG antibody cannot neutralize SARS-CoV-2 infectivity, e) protection against SARS-CoV-2 infection in a therapeutic animal model at a lower 50% effective dose (ED₅₀) than the bivalent IgG antibody, f) protection against SARS-CoV-2 infection in the therapeutic animal model under conditions where the bivalent reference IgG antibody cannot protect, g) protection against SARS-CoV-2 infection in a prophylactic animal model at a lower ED₅₀ than the bivalent IgG antibody, h) protection against SARS-CoV-2 infection in the prophylactic animal model under conditions where the bivalent reference IgG antibody cannot protect, or i) any combination thereof.

In certain embodiments the provided multimeric molecule can neutralize infectivity SARS-CoV-2 at a lower EC₅₀ than the bivalent reference IgG antibody or can neutralize infectivity of SARS-CoV-2 under conditions where the bivalent reference IgG antibody cannot neutralize. In certain embodiments the EC₅₀ is at least two-fold, at least five-fold, at least ten-fold, at least fifty-fold, at least 100-fold, at least 500-fold, or at least 1000-fold lower than the EC₅₀ of the bivalent IgG antibody. In certain embodiments the provided multimeric binding molecule can neutralize SARS-CoV-2 infectivity under conditions where the bivalent reference IgG antibody cannot neutralize SARS-CoV-2 infectivity. In certain embodiments those conditions can include neutralization of an antibody-resistant variant of SARS-CoV-2 where the bivalent reference IgG antibody cannot neutralize. In certain embodiments the antibody resistant variant of SARS-CoV-2 includes an “escape mutant” of a SARS-CoV-2 virus that arose following contact with the bivalent reference IgG antibody.

In certain embodiments the provided multimeric binding molecule can confer protection against SARS-CoV-2 infection in a therapeutic or prophylactic animal model at a lower 50% effective dose (ED₅₀) than the bivalent reference IgG antibody. In certain embodiments the provided multimeric binding molecule can confer protection against SARS-CoV-2 infection in a therapeutic or prophylactic animal model under conditions where the bivalent reference IgG antibody cannot protect. Conditions where the bivalent reference IgG antibody cannot protect against SARS-CoV-2 infection include, for example, a virus challenge with an antibody-resistant variant of SARS-CoV-2, for example, an “escape mutant” of a SARS-CoV-2 virus that arose following contact with the bivalent reference IgG antibody.

In certain embodiments the provided multimeric binding molecule can reduce, inhibit, or block the SARS-CoV-2 S protein from binding to ACE2 at a lower EC₅₀ than the bivalent reference IgG antibody or can reduce, inhibit, or block the SARS-CoV-2 S protein from binding to ACE2 under conditions where the bivalent reference IgG antibody cannot reduce, inhibit, or block the SARS-CoV-2 S protein from binding to ACE2.

In certain embodiments the immunoglobulin antigen-binding domains of the provided multimeric binding molecule are human immunoglobulin antigen-binding domains. In certain embodiments each binding unit of the provided multimeric binding molecule includes two heavy chains that each include the VH and two light chains that each include the VL. In certain embodiments the provided multimeric binding molecule includes two or four bivalent IgA or

IgA-like binding units and a J chain or functional fragment or variant thereof, where each binding unit includes two IgA heavy chain constant regions or multimerizing fragments or variants thereof, each including an IgA Cα3 domain and an IgA tailpiece domain. In certain embodiments the multimeric binding molecule is a dimeric binding molecule that includes two bivalent IgA or IgA-like binding units. In certain embodiments each IgA heavy chain constant region or multimerizing fragment or variant thereof further includes a Cα1 domain, a Cα2 domain, an IgA hinge region, or any combination thereof. In certain embodiments the IgA heavy chain constant regions or multimerizing fragments or variants thereof are human IgA constant regions including, e.g., the human IgA1 constant region amino acid sequence of SEQ ID NO: 3, the human IgA2 constant region amino acid sequence of SEQ ID NO: 4, or a multimerizing fragment or variant of SEQ ID NO: 3 or SEQ ID NO: 4. In certain embodiments each binding unit includes two IgA heavy chains each including a VH situated amino terminal to the IgA constant region or multimerizing fragment thereof, and two immunoglobulin light chains each including a VL situated amino terminal to an immunoglobulin light chain constant region.

In certain embodiments the provided multimeric binding molecule includes five or six bivalent IgM or IgM-like binding units, where each binding unit includes two IgM heavy chain constant regions or multimerizing fragments or variants thereof, each including an IgM Cμ4 and IgM tailpiece domain. In certain embodiments each IgM heavy chain constant region or multimerizing fragment or variant thereof can further include a Cμ1 domain, a Cμ2 domain, a Cμ3 domain, or any combination thereof. In certain embodiments the IgM heavy chain constant regions or multimerizing fragments or variants thereof are human IgM constant regions that can each include, for example, the amino acid sequence SEQ ID NO: 1, SEQ ID NO: 2, or a multimerizing fragment or variant thereof. In certain embodiments each binding unit includes two IgM heavy chains each including a VH situated amino terminal to the IgM constant region or multimerizing fragment or variant thereof, and two immunoglobulin light chains each including a VL situated amino terminal to an immunoglobulin light chain constant region. In certain embodiments the IgM constant regions each include one or more amino acid substitutions relative to a wild-type human IgM constant region at positions corresponding to amino acids 310, 311, 313, and/or 315 of SEQ ID NO: 1 or SEQ ID NO: 2, and where the multimeric binding molecule exhibits reduced complement-dependent cytotoxicity (CDC) activity to cells in the presence of complement, relative to a reference binding molecule that is identical except for the one or more amino acid substitutions. In certain embodiments the IgM constant regions each include one or more substitutions at positions corresponding to N46, N209, N272, or N440 of SEQ ID NO: 1 or SEQ ID NO: 2, where the one or more amino acid substitutions prevent asparagine (N)-linked glycosylation. In certain embodiments the provided multimeric binding molecule is pentameric, and further includes a J-chain or functional fragment or variant thereof.

In certain embodiments, the provided multimeric binding molecule can transport across vascular endothelial cells via J-chain binding to the polymeric Ig receptor (PIgR). In certain embodiments the provided multimeric binding molecule further includes a secretory component, or fragment or variant thereof.

In certain embodiments, the J-chain or functional fragment or variant thereof of the provided multimeric binding molecule further includes a heterologous polypeptide, where the heterologous polypeptide is directly or indirectly fused to the J-chain or functional fragment or variant thereof, for example, via a peptide linker. In certain embodiments a heterologous polypeptide can be fused to the N-terminus of the J-chain or fragment or variant thereof, the C-terminus of the J-chain or fragment or variant thereof, or to both the N-terminus and C-terminus of the J-chain or fragment or variant thereof, where the heterologous polypeptides fused to both the N-terminus and C-terminus can be the same or different. In certain embodiments the heterologous polypeptide can influence the absorption, distribution, metabolism and/or excretion (ADME) of the multimeric binding molecule. For example, the heterologous polypeptide can be an albumin or an albumin binding domain, e.g., human serum albumin.

This disclosure further provides a composition that includes the provided multimeric binding molecule. This disclosure further provides a composition that includes two or more nonidentical multimeric binding molecules as provided herein, where the two or more multimeric binding molecules bind to different epitopes of the SARS-CoV-2 spike (S) protein receptor binding domain (RBD).

The disclosure further provides a polynucleotide that includes a nucleic acid sequence that encodes a polypeptide subunit of the provided multimeric binding molecule, a vector that includes the polynucleotide, and/or a host cell that includes the polynucleotide or the vector, where the host cell can express the provided multimeric binding molecule.

The disclosure further provides a method of producing the provided multimeric binding molecule, where the method includes culturing the provided host cell and recovering the multimeric binding molecule. The provided method can further include contacting the multimeric binding molecule with a secretory component, or fragment or variant thereof.

The disclosure further provides a method for treating SARS-CoV-2 infection in a subject, where the method includes administering to a subject in need of treatment an effective amount of the provided multimeric binding molecule, where the multimeric binding molecule has greater antiviral potency against SARS-CoV-2 than a bivalent reference IgG antibody that includes two of the binding domains that specifically bind to the SARS-CoV-2 S protein RBD. The disclosure further provides a method for preventing SARS-CoV-2 infection in a subject, where the method includes administering to a subject susceptible to SARS-CoV-2 infection an effective amount of the provided multimeric binding molecule, where the multimeric binding molecule has greater antiviral potency against SARS-CoV-2 than a bivalent reference IgG antibody that includes two of the binding domains that specifically bind to the SARS-CoV-2 S protein RBD. In certain embodiments the SARS-CoV-2 infection is coronavirus disease 2019 (COVID-19). In certain embodiments the subject is human. In certain embodiments the administration can be intravenous, subcutaneous, intramuscular, intranasal, and/or inhalation administration. In one embodiment the administration includes intranasal administration. In certain embodiments the administration includes inhalation administration. In certain embodiments the administration includes intravenous infusion.

### BRIEF DESCRIPTION OF THE DRAWINGS/FIGURES

FIGS. 1A-1G show binding of anti-SARS-CoV2-06 IgM, IgA1, IgA2m2, and IgG (FIG. 1A), anti-SARS-CoV2-09 IgM, IgA1, IgA2m2, and IgG (FIG. 1B), anti-SARS-CoV2-12 IgM, IgA1, IgA2m2, and IgG (FIG. 1C), anti-SARS-CoV2-14 IgM, IgA1, IgA2m2, and IgG (FIG. 1D), anti-SARS-CoV2-16 IgM, IgA1, IgA2m2, and IgG (FIG. 1E), anti-SARS-CoV2-26 IgA1, IgA2m2, and IgG (FIG. 1F), CR3022 IgM, IgA1, IgA2m2, and IgG (FIG. 1G) to SARS-CoV-2 RBD in an ELISA assay.

FIG. 2A shows the ability of the disclosed anti-SARS-CoV2 antibodies and control antibodies to neutralize the infectivity of SARS-CoV-2 at a concentration of 1 μg/mL.

FIG. 2B shows the ability of the disclosed anti-SARS-CoV2 antibodies and control antibodies to neutralize the infectivity of SARS-CoV-2 at a concentration of 0.1 μg/mL.

FIG. 2C shows neutralization titration of SARS-CoV-2 live virus by anti-SARS-CoV2-06 IgM (squares) and anti-SARS-CoV2-06 IgG (circles).

FIG. 2D shows neutralization titration of SARS-CoV-2 live virus by anti-SARS-CoV2-14 IgM (squares) and anti-SARS-CoV2-14 IgG (circles).

FIG. 3A-3C shows the enhanced ability of selected anti-SARS-CoV2 IgM antibodies to block the binding of ACE2 to the RBD region of the SARS-CoV2 spike protein as compared to the corresponding IgG antibodies. FIG. 3A is a schematic diagram showing the BLI-based method for IgM or IgG blocking of the RBD-ACE2 interaction. FIG. 3B compares the dose dependent blocking of RBD binding to ACE2 by anti-SARS-CoV2-06 IgM (squares) and anti-SARS-CoV2-06 IgG (circles). FIG. 3C compares the dose dependent blocking of RBD binding to ACE2 by anti-SARS-CoV2-14 IgM (squares) and anti-SARS-CoV2-14 IgG (circles). The dashed lines in FIGS. 3B and 3C indicate 100% blocking.

FIG. 4A-4F show that anti-SARS-CoV2-14 IgM can neutralize a SARS-CoV2 escape mutant that arose upon exposure to anti-SARS-CoV2-14 IgG (E484A); an escape mutant that arose upon exposure to anti-SARS-CoV2-06 IgG (K444R), and an escape mutant comprising both mutations. FIGS. 4A-4C show virus neutralization by anti-SARS-CoV2-06 IgM (squares) and IgG (circles) against SARS-CoV2 with RBD mutations at K444R (arose upon exposure to anti-SARS-CoV2-06 IgG) (FIG. 4A), E484A (arose upon exposure to anti-SARS-CoV2-14 IgG) (FIG. 4B) and K444R+E484A (FIG. 4C). FIGS. 4D-4F show virus neutralization by anti-SARS-CoV2-14 IgM (squares) and IgG (circles) against SARS-CoV2 with RBD mutations at K444R (FIG. 4D), E484A (FIG. 4E) and K444R+E484A (FIG. 4F).

FIG. 5A-5H show that nasal delivery of anti-SARS-CoV2-14 IgM confers effective prophylactic and therapeutic protection from SARS-CoV-2 infection in three different studies in mice. For study #1, FIG. 5A is a schematic diagram showing a prophylactic pretreatment and challenge strategy, and FIG. 5B shows the results. Significance was calculated via the two-tailed unpaired t-test. “DPBS” is Dulbecco's phosphate-buffered saline. For study #2, FIG. 5C is a schematic diagram showing the prophylactic and therapeutic treatment strategies, FIG. 5D shows the results for prophylactic treatment, and FIG. 5E shows the results for therapeutic treatment. Significance was calculated via one-way ANOVA with Sidak's multiple comparisons. For study #3, FIG. 5F is a schematic diagram showing the prophylactic and therapeutic treatment strategies, FIG. 5G shows the results for prophylactic treatment with CoV2-14 IgM at lower doses compared to previously tested doses, and FIG. 5H shows comparative results for therapeutic treatment with CoV2-14 IgM and CoV2-14 IgG.

FIG. 6A-6F show biodistribution of CoV2-14 IgM in a mouse model. FIG. 6A is a schematic diagram showing bio-distribution evaluations of IgM-14 in mice by near-infrared fluorescence (NIRF) imaging. FIG. 6B shows representative live body images at indicated time points. FIGS. 6C-6F show quantitation of fluorescence signals in different organs at 24 hours (FIG. 6C), 48 hours (FIG. 6D), 96 hours (FIG. 6E) and 168 hours (FIG. 6F).

FIG. 7A-7J show that SARS-CoV2-14 IgM can neutralize emerging variants at higher potency than SARS-CoV2-14 IgG in a plaque reduction assay. Neutralization studies were performed using recombinant versions of the wild-type WA1 SARS-CoV-2 virus comprising spike proteins containing all of the reported mutations of variant viruses. FIG. 7A: Linear representation of SARS-CoV-2 spike protein showing some of the key subunits involved with mutations as well as antibodies, including the N-terminal domain (NTD), the receptor binding domain (RBD), receptor binding motif (RBM), S1/S2 region around the furin protease cleavage site, the S2 domain, and the transmembrane (TM) region at the C-terminus of S2. FIG. 7B: A complete list of all the World Health Organization (WHO) designated Variants of Concern (VOC) and the Variants of Interest (VOI) (as of Dec. 8, 2021) as well as the “original variant” D614G, and the mutations each variant carries in each of the major domains. All amino acids are noted by their single letter designation. Abbreviations: VOC, VOI, NTD, and RBD, as noted above; NC, not categorized; Deletions are noted by Δ followed by the deleted amino acids; Symbols: \hat{ } D614G was found in many sequences very soon after sequencing efforts began in early 2020; # the VOIs Epsilon, Theta, Eta, Kappa, Iota, and Zeta, were declassified as VOIs, so those names are provided parenthetically; @ mutant positions in parentheses (e.g., (S13I)) indicate mutations that are only sometimes associated with the variant listed. Data are from the World Health Organization: who.int/en/activities/tracking-SARS-CoV-2-variants/ (last visited on Dec. 14, 2021) and US Centers for Disease Control (US CDC) cdc.gov/coronavirus/2019-ncov/variants/variant-info.html (last visited on Dec. 14, 2021). FIG. 7C: neutralization of wild-type clinical strain USA-WA1/2020 (“WA1”); FIG. 7D: neutralization of WA1 comprising the B.1.1.7 “Alpha” variant spike protein; FIG. 7E: neutralization of WA1 comprising the P.1 “Gamma” variant spike protein; FIG. 7F: neutralization of WA1 comprising the B.1.351 “Beta” variant spike protein. FIG. 7G: neutralization of WA1 comprising the B.1.617.2 “Delta” variant spike protein. FIG. 7H: neutralization of WA1 comprising the C.37 “Lambda” variant spike protein. FIG. 7I: neutralization of WA1 comprising the B.1.621 “Mu” variant spike protein. FIG. 7J: neutralization of WA1 comprising the B.1.1.529 “Omicron” variant spike protein.

FIG. 8A-8B show that nasal delivery of anti-SARS-CoV2-14 IgM confers effective prophylactic and therapeutic protection from SARS-CoV-2 infection using alternative viral RNA load assay. FIG. 8A shows the results for prophylactic treatment, and FIG. 8B shows the results for therapeutic treatment. Significance was calculated via one-way ANOVA with Sidak's multiple comparisons.

FIG. 9A-9B show that nasal delivery of anti-SARS-CoV2-14 IgM confers effective therapeutic protection from infection with SARS-CoV-2 carrying the Gamma or Beta variant spike proteins. FIG. 9A shows the results for the Gamma variant, FIG. 9B shows the results for the Beta variant. Significance was calculated via one-way ANOVA with Sidak's multiple comparisons.

FIG. 10A-10E show that nasal delivery of anti-SARS-CoV2-14 IgM confers effective therapeutic protection from infection with SARS-CoV-2 carrying the Delta variant spike protein in the K18-hACE2 mouse model. FIG. 10A shows schematic of the in vivo study. FIG. 10B shows weight loss results upon infection with SARS-CoV-2 USA-WA1/2020. FIG. 10C shows viral RNA copies at day 7 for animals infected with SARS-CoV-2 USA-WA1/2020 and treated with anti-SARS-CoV2-14 IgM. FIG. 10D shows weight loss results upon infection with recombinant SARS-CoV-2 USA-WA1/2020 carrying the Delta variant spike protein. FIG. 10E shows viral RNA copies at day 7 for animals infected with recombinant SARS-CoV-2 USA-WA1/2020 carrying the Delta variant spike protein and treated with anti-SARS-CoV2-14 IgM. Significance was calculated via one-way ANOVA with Sidak's multiple comparisons.

### DETAILED DESCRIPTION

### Definitions

As used herein, the term “a” or “an” entity refers to one or more of that entity; for example, “a binding molecule,” is understood to represent one or more binding molecules. As such, the terms “a” (or “an”), “one or more,” and “at least one” can be used interchangeably herein.

Furthermore, “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Unless defined otherwise, technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, amino acid sequences are written left to right in amino to carboxy orientation. The headings provided herein are not limitations of the various embodiments or embodiments of the disclosure, which can be had by reference to the specification as a whole. Accordingly, the terms defined immediately below are more fully defined by reference to the specification in its entirety.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids and does not refer to a specific length of the product. Thus, peptides, dipeptides, tripeptides, oligopeptides, “protein,” “amino acid chain,” or any other term used to refer to a chain or chains of two or more amino acids are included within the definition of “polypeptide,” and the term “polypeptide” can be used instead of any of these terms. The term “polypeptide” is also intended to refer to the products of post-expression modifications of the polypeptide, including without limitation glycosylation, acetylation, phosphorylation, amidation, and derivatization by known protecting/blocking groups, proteolytic cleavage, or modification by non-naturally occurring amino acids. A polypeptide can be derived from a biological source or produced by recombinant technology but is not necessarily translated from a designated nucleic acid sequence. It can be generated in any manner, including by chemical synthesis.

A polypeptide as disclosed herein can be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides can have a defined three-dimensional structure, although they do not necessarily have such structure. Polypeptides with a defined three-dimensional structure are referred to as folded, and polypeptides which do not possess a defined three-dimensional structure, but rather can adopt many different conformations and are referred to as unfolded. As used herein, the term glycoprotein refers to a protein coupled to at least one carbohydrate moiety that is attached to the protein via an oxygen-containing or a nitrogen-containing side chain of an amino acid, e.g., a serine or an asparagine. Asparagine (N)-linked glycans are described in more detail elsewhere in this disclosure.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated as disclosed herein, as are native or recombinant polypeptides which have been separated, fractionated, or partially or substantially purified by any suitable technique.

As used herein, the term “a non-naturally occurring polypeptide” or any grammatical variants thereof, is a conditional definition that explicitly excludes, but only excludes, those forms of the polypeptide that are, or might be, determined or interpreted by a judge or an administrative or judicial body, to be “naturally-occurring.”

Other polypeptides disclosed herein are fragments, derivatives, analogs, or variants of the foregoing polypeptides, and any combination thereof. The terms “fragment,” “variant,” “derivative” and “analog” as disclosed herein include any polypeptides which retain at least some of the properties of the corresponding native antibody or polypeptide, for example, specifically binding to an antigen. Fragments of polypeptides include, for example, proteolytic fragments, as well as deletion fragments, in addition to specific antibody fragments discussed elsewhere herein. Variants of, e.g., a polypeptide include fragments as described above, and also polypeptides with altered amino acid sequences due to amino acid substitutions, deletions, or insertions. In certain embodiments, variants can be non-naturally occurring. Non-naturally occurring variants can be produced using art-known mutagenesis techniques. Variant polypeptides can comprise conservative or non-conservative amino acid substitutions, deletions, or additions. Derivatives are polypeptides that have been altered so as to exhibit additional features not found on the original polypeptide. Examples include fusion proteins. As used herein a “derivative” of a polypeptide can also refer to a subject polypeptide having one or more amino acids chemically derivatized by reaction of a functional side group. Also included as “derivatives” are those polypeptides that contain one or more derivatives of the twenty standard amino acids. For example, 4-hydroxyproline can be substituted for proline; 5-hydroxylysine can be substituted for lysine; 3-methylhistidine can be substituted for histidine; homoserine can be substituted for serine; and ornithine can be substituted for lysine.

A “conservative amino acid substitution” is one in which one amino acid is replaced with another amino acid having a similar side chain. Families of amino acids having similar side chains have been defined in the art, including basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. In certain embodiments, conservative substitutions in the sequences of the polypeptides, binding molecules, and antibodies of the present disclosure do not abrogate the binding of the polypeptide, binding molecule, or antibody containing the amino acid sequence, to the antigen to which the antibody binds. Methods of identifying nucleotide and amino acid conservative substitutions which do not eliminate antigen-binding are well-known in the art (see, e.g., Brummell et al., Biochem. 32: 1180-1 187 (1993); Kobayashi et al., Protein Eng. 12:879-884 (1999); and Burks et al., Proc. Natl. Acad. Sci. USA 94:412-417 (1997)).

The term “polynucleotide” is intended to encompass a singular nucleic acid as well as plural nucleic acids and refers to an isolated nucleic acid molecule or construct, e.g., messenger RNA (mRNA), cDNA, or plasmid DNA (pDNA). A polynucleotide can comprise a conventional phosphodiester bond or a non-conventional bond (e.g., an amide bond, such as found in peptide nucleic acids (PNA)). The terms “nucleic acid” or “nucleic acid sequence” refer to any one or more nucleic acid segments, e.g., DNA or RNA fragments, present in a polynucleotide.

By an “isolated” nucleic acid or polynucleotide is intended any form of the nucleic acid or polynucleotide that is separated from its native environment. For example, gel-purified polynucleotide, or a recombinant polynucleotide encoding a polypeptide contained in a vector would be considered to be “isolated.” Also, a polynucleotide segment, e.g., a PCR product, which has been engineered to have restriction sites for cloning is considered to be “isolated.” Further examples of an isolated polynucleotide include recombinant polynucleotides maintained in heterologous host cells or purified (partially or substantially) polynucleotides in a non-native solution such as a buffer or saline. Isolated RNA molecules include in vivo or in vitro RNA transcripts of polynucleotides, where the transcript is not one that would be found in nature. Isolated polynucleotides or nucleic acids further include such molecules produced synthetically. In addition, polynucleotide or a nucleic acid can be or can include a regulatory element such as a promoter, ribosome binding site, or a transcription terminator.

As used herein, the term “a non-naturally occurring polynucleotide” or any grammatical variants thereof, is a conditional definition that explicitly excludes, but only excludes, those forms of the nucleic acid or polynucleotide that are, or might be, determined or interpreted by a judge, or an administrative or judicial body, to be “naturally-occurring.”

As used herein, a “coding region” is a portion of nucleic acid which consists of codons translated into amino acids. Although a “stop codon” (TAG, TGA, or TAA) is not translated into an amino acid, it can be considered to be part of a coding region, but any flanking sequences, for example promoters, ribosome binding sites, transcriptional terminators, introns, and the like, are not part of a coding region. Two or more coding regions can be present in a single polynucleotide construct, e.g., on a single vector, or in separate polynucleotide constructs, e.g., on separate (different) vectors. Furthermore, any vector can contain a single coding region, or can comprise two or more coding regions, e.g., a single vector can separately encode an immunoglobulin heavy chain variable region and an immunoglobulin light chain variable region. In addition, a vector, polynucleotide, or nucleic acid can include heterologous coding regions, either fused or unfused to another coding region. Heterologous coding regions include without limitation, those encoding specialized elements or motifs, such as a secretory signal peptide or a heterologous functional domain.

In certain embodiments, the polynucleotide or nucleic acid is DNA. In the case of DNA, a polynucleotide comprising a nucleic acid which encodes a polypeptide normally can include a promoter and/or other transcription or translation control elements operably associated with one or more coding regions. An operable association is when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter can be a cell-specific promoter that directs substantial transcription of the DNA in predetermined cells. Other transcription control elements, besides a promoter, for example enhancers, operators, repressors, and transcription termination signals, can be operably associated with the polynucleotide to direct cell-specific transcription.

A variety of transcription control regions are known to those skilled in the art. These include, without limitation, transcription control regions that function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit ß-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to ribosome binding sites, translation initiation and termination codons, and elements derived from picornaviruses (particularly an internal ribosome entry site, or IRES, also referred to as a CITE sequence).

In other embodiments, a polynucleotide can be RNA, for example, in the form of messenger RNA (mRNA), transfer RNA, or ribosomal RNA.

Polynucleotide and nucleic acid coding regions can be associated with additional coding regions which encode secretory or signal peptides, which direct the secretion of a polypeptide encoded by a polynucleotide as disclosed herein. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells can have a signal peptide fused to the N-terminus of the polypeptide, which is cleaved from the complete or “full length” polypeptide to produce a secreted or “mature” form of the polypeptide. In certain embodiments, the native signal peptide, e.g., an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, can be used. For example, the wild-type leader sequence can be substituted with the leader sequence of human tissue plasminogen activator (TPA) or mouse ß-glucuronidase.

As used herein, the term “binding molecule” refers in its broadest sense to a molecule that specifically binds to a receptor or target, e.g., an epitope or an antigenic determinant. As described further herein, a binding molecule can comprise one of more “binding domains,” e.g., “antigen-binding domains” described herein. A non-limiting example of a binding molecule is an antibody or antibody-like molecule as described in detail herein that retains antigen-specific binding. In certain embodiments a “binding molecule” comprises an antibody or antibody-like or antibody-derived molecule as described in detail herein.

As used herein, the terms “binding domain” or “antigen-binding domain” (can be used interchangeably) refer to a region of a binding molecule, e.g., an antibody or antibody-like, or antibody-derived molecule, that is necessary and sufficient to specifically bind to a target, e.g., an epitope, a polypeptide, a cell, or an organ. For example, an “Fv,” e.g., a heavy chain variable region and a light chain variable region of an antibody, either as two separate polypeptide subunits or as a single chain, is considered to be a “binding domain.” Other antigen-binding domains include, without limitation, a single domain heavy chain variable region (VHH) of an antibody derived from a camelid species, or six immunoglobulin complementarity determining regions (CDRs) expressed in a fibronectin scaffold. A “binding molecule,” e.g., an “antibody” as described herein can include one, two, three, four, five, six, seven, eight, nine, ten, eleven, twelve, or more “antigen-binding domains.”

The terms “antibody” and “immunoglobulin” can be used interchangeably herein. An antibody (or a fragment, variant, or derivative thereof as disclosed herein, e.g., an IgM-like antibody) includes at least the variable domain of a heavy chain (e.g., from a camelid species) or at least the variable domains of a heavy chain and a light chain. Basic immunoglobulin structures in vertebrate systems are relatively well understood. See, e.g., Harlow et al., Antibodies: A Laboratory Manual, (Cold Spring Harbor Laboratory Press, 2nd ed. 1988). Unless otherwise stated, the term “antibody” encompasses anything ranging from a small antigen-binding fragment of an antibody to a full sized antibody, e.g., an IgG antibody that includes two complete heavy chains and two complete light chains, an IgA antibody that includes four complete heavy chains and four complete light chains and includes a J-chain and/or a secretory component, or an IgM-derived binding molecule, e.g., an IgM antibody or IgM-like antibody, that includes ten or twelve complete heavy chains and ten or twelve complete light chains and optionally includes a J-chain or functional fragment or variant thereof.

The term “immunoglobulin” comprises various broad classes of polypeptides that can be distinguished biochemically. Those skilled in the art will appreciate that heavy chains are classified as gamma, mu, alpha, delta, or epsilon, (γ, μ, α, δ, ε) with some subclasses among them (e.g., γ1-γ4 or α1-α2)). It is the nature of this chain that determines the “isotype” of the antibody as IgG, IgM, IgA IgD, or IgE, respectively. The immunoglobulin subclasses (subtypes) e.g., IgG₁, IgG₂, IgG₃, IgG₄, IgA₁, IgA₂, etc. are well characterized and are known to confer functional specialization. Modified versions of each of these immunoglobulins are readily discernible to the skilled artisan in view of the instant disclosure and, accordingly, are within the scope of this disclosure.

Light chains are classified as either kappa or lambda (κ, λ). Each heavy chain class can be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are expressed, e.g., by hybridomas, B cells or genetically engineered host cells. In the heavy chain, the amino acid sequences run from an N-terminus at the forked ends of the Y configuration to the C-terminus at the bottom of each chain. The basic structure of certain antibodies, e.g., IgG antibodies, includes two heavy chain subunits and two light chain subunits covalently connected via disulfide bonds to form a “Y” structure, also referred to herein as an “H2L2” structure, or a “binding unit.”

The term “binding unit” is used herein to refer to the portion of a binding molecule, e.g., an antibody, antibody-like molecule, or antibody-derived molecule, antigen-binding fragment thereof, or multimerizing fragment thereof, which corresponds to a standard “H2L2” immunoglobulin structure, i.e., two heavy chains or fragments thereof and two light chains or fragments thereof. In certain embodiments, e.g., where the binding molecule is a bivalent IgG antibody or antigen-binding fragment thereof, the terms “binding molecule” and “binding unit” are equivalent. Such binding molecules are also referred to herein as “monomeric.” In other embodiments, e.g., where the binding molecule is a “multimeric binding molecule,” e.g., a dimeric or tetrameric IgA antibody, a dimeric or tetrameric IgA-like antibody, a dimeric or tetrameric IgA-derived binding molecule, a pentameric or hexameric IgM antibody, a pentameric or hexameric IgM-like antibody, or a pentameric or hexameric IgM-derived binding molecule or any derivative thereof, the binding molecule comprises two or more “binding units.” Two in the case of an IgA dimer, four in the case of an IgA tetramer, or five or six in the case of an IgM pentamer or hexamer, respectively. A binding unit need not include full-length antibody heavy and light chains, but will typically be bivalent, i.e., will include two “antigen-binding domains,” as defined above. As used herein, certain binding molecules provided in this disclosure are “dimeric,” and include two bivalent binding units that include IgA constant regions or multimerizing fragments thereof. Certain binding molecules provided in this disclosure are “pentameric” or “hexameric,” and include five or six bivalent binding units that include IgM constant regions or multimerizing fragments or variants thereof. A binding molecule, e.g., an antibody or antibody-like molecule or antibody-derived binding molecule, comprising two or more, e.g., two, five, or six binding units, is referred to herein as “multimeric.”

The term “J-chain” as used herein refers to the J-chain of IgM or IgA antibodies of any animal species, any functional fragment thereof, derivative thereof, and/or variant thereof, including a mature human J-chain, the amino acid sequence of which is presented as SEQ ID NO: 7. Various J-chain variants and modified J-chain derivatives are disclosed herein. As persons of ordinary skill in the art will recognize, “a functional fragment” or “a functional variant” includes those fragments and variants that can associate with IgM heavy chain constant regions to form a pentameric IgM antibody or can associate with IgA heavy chain constant regions to form a dimeric IgA antibody.

The term “modified J-chain” is used herein to refer to a derivative of a J-chain polypeptide comprising a heterologous moiety, e.g., a heterologous polypeptide, e.g., an extraneous binding domain or functional domain introduced into or attached to the J-chain sequence. The introduction can be achieved by any means, including direct or indirect fusion of the heterologous polypeptide or other moiety or by attachment through a peptide or chemical linker. The term “modified human J-chain” encompasses, without limitation, a native sequence human J-chain comprising the amino acid sequence of SEQ ID NO: 7 or functional fragment thereof, or functional variant thereof, modified by the introduction of a heterologous moiety, e.g., a heterologous polypeptide, e.g., an extraneous binding domain. In certain embodiments the heterologous moiety does not interfere with efficient polymerization of IgM into a pentamer or IgA into a multimer, e.g., a dimer or tetramer, and binding of such polymers to a target. Exemplary modified J-chains can be found, e.g., in U.S. Pat. Nos. 9,951,134, 10,400,038, and 10,618,978, and in U.S. Patent Application Publication No. US-2019-0185570, each of which is incorporated herein by reference in its entirety.

As used herein the term “IgM-derived binding molecule” refers collectively to native IgM antibodies, IgM-like antibodies, as well as other IgM-derived binding molecules comprising non-antibody binding and/or functional domains instead of an antibody antigen binding domain or subunit thereof, and any fragments, e.g., multimerizing fragments, variants, or derivatives thereof.

As used herein, the term “IgM-like antibody” refers generally to a variant antibody or antibody-derived binding molecule that still retains the ability to form hexamers or pentamers, e.g., in association with a J-chain. An IgM-like antibody or other IgM-derived binding molecule typically includes at least the Cμ4-tp domains of the IgM constant region but can include heavy chain constant region domains from other antibody isotypes, e.g., IgG, from the same species or from a different species. An IgM-like antibody or other IgM-derived binding molecule can likewise be an antibody fragment in which one or more constant regions are deleted, as long as the IgM-like antibody is capable of forming hexamers and/or pentamers. Thus, an IgM-like antibody or other IgM-derived binding molecule can be, e.g., a hybrid IgM/IgG antibody or can be a “multimerizing fragment” of an IgM antibody.

As used herein the term “IgA-derived binding molecule” refers collectively to native IgA antibodies, IgA-like antibodies, as well as other IgA-derived binding molecules comprising non-antibody binding and/or functional domains instead of an antibody antigen binding domain or subunit thereof, and any fragments, e.g., multimerizing fragments, variants, or derivatives thereof.

As used herein, the term “IgA-like antibody” refers generally to a variant antibody or antibody-derived binding molecule that still retains the ability to form multimers, e.g., dimers, trimers, tetramers, and/or pentamers e.g., dimers and/or tetramers, e.g., in association with a J-chain. An IgA-like antibody or other IgA-derived binding molecule typically includes at least the Cα3-tp domains of the IgA constant region but can include heavy chain constant region domains from other antibody isotypes, e.g., IgG, from the same species or from a different species. An IgA-like antibody or other IgA-derived binding molecule can likewise be an antibody fragment in which one or more constant regions are deleted, as long as the IgA-like antibody is capable of forming multimers, e.g., dimers and/or tetramers. Thus, an IgA-like antibody or other IgA-derived binding molecule can be, e.g., a hybrid IgA/IgG antibody or can be a “multimerizing fragment” of an IgA antibody.

The terms “valency,” “bivalent,” “multivalent” and grammatical equivalents, refer to the number of binding domains, e.g., antigen-binding domains in given binding molecule, e.g., antibody, antibody-derived, or antibody-like molecule, or in a given binding unit. As such, the terms “bivalent”, “tetravalent”, and “hexavalent” in reference to a given binding molecule, e.g., an IgM antibody, IgM-like antibody, other IgM-derived binding molecule, or multimerizing fragment thereof, denote the presence of two antigen-binding domains, four antigen-binding domains, and six antigen-binding domains, respectively. A typical IgM antibody, IgM-like antibody, or other IgM-derived binding molecule, where each binding unit is bivalent, can have 10 or 12 valencies. A bivalent or multivalent binding molecule, e.g., antibody or antibody-derived molecule, can be monospecific, i.e., all of the antigen-binding domains are the same, or can be bispecific or multispecific, e.g., where two or more antigen-binding domains are different, e.g., bind to different epitopes on the same antigen, or bind to entirely different antigens.

The term “epitope” includes any molecular determinant capable of specific binding to an antigen-binding domain of an antibody, antibody-like, or antibody-derived molecule. In certain embodiments, an epitope can include chemically active surface groupings of molecules such as amino acids, sugar side chains, phosphoryl groups, or sulfonyl groups, and, in certain embodiments, can have three-dimensional structural characteristics, and or specific charge characteristics. An epitope is a region of a target that is bound by an antigen-binding domain of an antibody.

The term “target” is used in the broadest sense to include substances that can be bound by a binding molecule, e.g., antibody, antibody-like, or antibody-derived molecule. A target can be, e.g., a polypeptide, a nucleic acid, a carbohydrate, a lipid, or other molecule, or a minimal epitope on such molecule. Moreover, a “target” can, for example, be a cell, an organ, or an organism, e.g., an animal, plant, microbe, or virus, that comprises an epitope that can be bound by a binding molecule, e.g., antibody, antibody-like, or antibody-derived molecule.

Both the light and heavy chains of antibodies, antibody-like, or antibody-derived molecules are divided into regions of structural and functional homology. The terms “constant” and “variable” are used functionally. In this regard, it will be appreciated that the variable domains of both the variable light (VL) and variable heavy (VH) chain portions determine antigen recognition and specificity. Conversely, the constant region domains of the light chain (CL) and the heavy chain (e.g., CH1, CH2, CH3, or CH4) confer biological properties such as secretion, transplacental mobility, Fc receptor binding, complement binding, and the like. By convention, the numbering of the constant region domains increases as they become more distal from the antigen-binding site or amino-terminus of the antibody. The N-terminal portion is a variable region and at the C-terminal portion is a constant region; the CH3 (or CH4, e.g., in the case of IgM) and CL domains actually comprise the carboxy-terminus of the heavy and light chain, respectively.

A “full length IgM antibody heavy chain” is a polypeptide that includes, in N-terminal to C-terminal direction, an antibody heavy chain variable domain (VH), an antibody heavy chain constant domain 1 (CM1 or Cμ1), an antibody heavy chain constant domain 2 (CM2 or Cμ2), an antibody heavy chain constant domain 3 (CM3 or Cμ3), and an antibody heavy chain constant domain 4 (CM4 or Cμ4) that can include a tailpiece.

A “full length IgA antibody heavy chain” is a polypeptide that includes, in N-terminal to C-terminal direction, an antibody heavy chain variable domain (VH), an antibody heavy chain constant domain 1 (CA1 or Cα1), an IgA hinge region, an antibody heavy chain constant domain 2 (CA2 or Cα2), and an antibody heavy chain constant domain 3 (CA3 or Cα3) that can include an IgA tailpiece.

As indicated above, variable region(s) allow a binding molecule, e.g., antibody, antibody-like, or antibody-derived molecule, to selectively recognize and specifically bind epitopes on antigens. That is, the VL domain and VH domain, or subset of the complementarity determining regions (CDRs), of a binding molecule, e.g., an antibody, antibody-like, or antibody-derived molecule, combine to form the antigen-binding domain. More specifically, an antigen-binding domain can be defined by three CDRs on each of the VH and VL chains. Certain antibodies form larger structures. For example, IgA can form a molecule that includes two H2L2 binding units and a J-chain covalently connected via disulfide bonds, which can be further associated with a secretory component, and IgM can form a pentameric or hexameric molecule that includes five or six H2L2 binding units and optionally a J-chain covalently connected via disulfide bonds.

The six “complementarity determining regions” or “CDRs” present in an antibody antigen-binding domain are short, non-contiguous sequences of amino acids that are specifically positioned to form the antigen-binding domain as the antibody assumes its three-dimensional configuration in an aqueous environment. The remainder of the amino acids in the antigen-binding domain, referred to as “framework” regions, show less inter-molecular variability. The framework regions largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions. The antigen-binding domain formed by the positioned CDRs defines a surface complementary to the epitope on the immunoreactive antigen. This complementary surface promotes the non-covalent binding of the antibody to its cognate epitope. The amino acids that make up the CDRs and the framework regions, respectively, can be readily identified for any given heavy or light chain variable region by one of ordinary skill in the art, since they have been defined in various different ways (see, “Sequences of Proteins of Immunological Interest,” Kabat, E., et al., U.S. Department of Health and Human Services, (1983); and Chothia and Lesk, J. Mol. Biol., 196:901-917 (1987), which are incorporated herein by reference in their entireties).

In the case where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. These particular regions have been described, for example, by Kabat et al., U.S. Dept. of Health and Human Services, “Sequences of Proteins of Immunological Interest” (1983) and by Chothia et al., J. Mol. Biol. 196:901-917 (1987), which are incorporated herein by reference. The Kabat and Chothia definitions include overlapping or subsets of amino acids when compared against each other. Nevertheless, application of either definition (or other definitions known to those of ordinary skill in the art) to refer to a CDR of an antibody or variant thereof is intended to be within the scope of the term as defined and used herein, unless otherwise indicated. The appropriate amino acids which encompass the CDRs as defined by each of the above cited references are set forth below in Table 1 as a comparison. The exact amino acid numbers which encompass a particular CDR will vary depending on the sequence and size of the CDR. Those skilled in the art can routinely determine which amino acids comprise a particular CDR given the variable region amino acid sequence of the antibody.

TABLE 1
CDR Definitions*

| | Kabat | Chothia |
|---|---|---|
| VH CDR1 | 31-35 | 26-32 |
| VH CDR2 | 50-65 | 52-58 |
| VH CDR3 | 95-102 | 95-102 |
| VL CDR1 | 24-34 | 26-32 |
| VL CDR2 | 50-56 | 50-52 |
| VL CDR3 | 89-97 | 91-96 |

*Numbering of all CDR definitions in Table 1 is according to the numbering conventions set forth by Kabat et al. (see below).

Antibody variable domains can also be analyzed, e.g., using the IMGT information system (imgt.cines.fr (visited Jan. 24, 2021)) (IMGT®/V-Quest) to identify variable region segments, including CDRs. See, e.g., Brochet et al., Nucl. Acids Res. 36:W503-508, 2008. IMGT uses a different numbering system than Kabat. See, e.g., Lefranc, M.-P. et al., Dev. Comp. Immunol. 27:55-77 (2003). Correspondences are listed, for example, at imgt.org/IMGTScientificChart/Numbering/CDR1-IMGTgaps.html (visited Jan. 24, 2021).

Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless use of the Kabat numbering system is explicitly noted, however, consecutive numbering is used for all amino acid sequences in this disclosure.

The Kabat numbering system for the human IgM constant domain can be found in Kabat, et. al. “Tabulation and Analysis of Amino acid and nucleic acid Sequences of Precursors, V-Regions, C-Regions, J-Chain, T-Cell Receptors for Antigen, T-Cell Surface Antigens, β-2 Microglobulins, Major Histocompatibility Antigens, Thy-1, Complement, C-Reactive Protein, Thymopoietin, Integrins, Post-gamma Globulin, α-2 Macroglobulins, and Other Related Proteins,” U.S. Dept. of Health and Human Services (1991). IgM constant regions can be numbered sequentially (i.e., amino acid #1 starting with the first amino acid of the constant region, or by using the Kabat numbering scheme. A comparison of the numbering of two alleles of the human IgM constant region sequentially (presented herein as SEQ ID NO: 1 (allele IGHM*03) and SEQ ID NO: 2 (allele IGHM*04)) and by the Kabat system is set out below. The underlined amino acid residues are not accounted for in the Kabat system (“X,” double underlined below, can be serine (S) (SEQ ID NO: 1) or glycine (G) (SEQ ID NO: 2)):

Sequential (SEQ ID NO: 1 or SEQ ID NO: 2)/KABAT
numbering key for IgM heavy chain
(SEQ ID NO: 107)

| | |
|---|---|
| 1/127 | GSASAPTLFP LVSCENSPSD TSSVAVGCLA |
| | QDFLPDSITF SWKYKNNSDI |
| 51/176 | SSTRGFPSVL RGGKYAATSQ VLLPSKDVMQ |
| | GTDEHVVCKV QHPNGNKEKN |
| 101/226 | VPLPVIAELP PKVSVFVPPR DGFFGNPRKS |
| | KLICQATGFS PRQIQVSWLR |
| 151/274 | EGKQVGSGVT TDQVQAEAKE SGPTTYKVTS |
| | TLTIKESDWL XQSMFTCRVD |
| 201/324 | HRGLTFQQNA SSMCVPDQDT AIRVFAIPPS |
| | FASIFLTKST KLTCLVTDLT |
| 251/374 | TYDSVTISWT RQNGEAVKTH TNISESHPNA |
| | TFSAVGEASI CEDDWNSGER |
| 301/424 | FTCTVTHTDL PSPLKQTISR PKGVALHRPD |
| | VYLLPPAREQ LNLRESATIT |
| 351/474 | CLVTGFSPAD VFVQWMQRGQ PLSPEKYVTS |
| | APMPEPQAPG RYFAHSILTV |
| 401/524 | SEEEWNTGET YTCVVAHEAL PNRVTERTVD |
| | KSTGKPTLYN VSLVMSDTAG |
| 451/574 | TCY |

Binding molecules, e.g., antibodies, antibody-like, or antibody-derived molecules, antigen-binding fragments, variants, or derivatives thereof, and/or multimerizing fragments thereof include, but are not limited to, polyclonal, monoclonal, human, humanized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a VL or VH domain, fragments produced by a Fab expression library. ScFv molecules are known in the art and are described, e.g., in U.S. Pat. No. 5,892,019.

By “specifically binds,” it is generally meant that a binding molecule, e.g., an antibody or fragment, variant, or derivative thereof binds to an epitope via its antigen-binding domain, and that the binding entails some complementarity between the antigen-binding domain and the epitope. According to this definition, a binding molecule, e.g., antibody, antibody-like, or antibody-derived molecule, is said to “specifically bind” to an epitope when it binds to that epitope, via its antigen-binding domain more readily than it would bind to a random, unrelated epitope. The term “specificity” is used herein to qualify the relative affinity by which a certain binding molecule binds to a certain epitope. For example, binding molecule “A” can be deemed to have a higher specificity for a given epitope than binding molecule “B,” or binding molecule “A” can be said to bind to epitope “C” with a higher specificity than it has for related epitope “D.”

A binding molecule, e.g., an antibody or fragment, variant, or derivative thereof disclosed herein can be said to bind a target antigen with an off rate (k(off)) of less than or equal to 5×10⁻² sec⁻¹, 10⁻² sec⁻¹, 5×10⁻³ sec⁻¹, 10⁻³ sec⁻¹, 5×10⁻⁴ sec⁻¹, 10⁻⁴ sec⁻¹, 5×10⁻⁵ sec⁻¹, or 10⁻⁵ sec⁻¹ 5×10⁻⁶ sec⁻¹, 10⁻⁶ sec⁻¹, 5×10⁻⁷ sec⁻¹ or 10⁻⁷ sec⁻¹.

A binding molecule, e.g., an antibody or antigen-binding fragment, variant, or derivative disclosed herein can be said to bind a target antigen with an on rate (k(on)) of greater than or equal to 10³ M⁻¹ sec⁻¹, 5×10³ M⁻¹ sec⁻¹, 10⁴ M⁻¹ sec⁻¹, 5×10⁴ M⁻¹ sec⁻¹, 10⁵ M⁻¹ sec⁻¹, 5×10⁵ M⁻¹ sec⁻¹, 10⁶ M⁻¹ sec⁻¹, or 5×10⁶ M⁻¹ sec⁻¹ or 10⁷ M⁻¹ sec⁻¹.

A binding molecule, e.g., an antibody or fragment, variant, or derivative thereof is said to competitively inhibit binding of a reference antibody or antigen-binding fragment to a given epitope if it preferentially binds to that epitope to the extent that it blocks, to some degree, binding of the reference antibody or antigen-binding fragment to the epitope. Competitive inhibition can be determined by any method known in the art, for example, competition ELISA assays. A binding molecule can be said to competitively inhibit binding of the reference antibody or antigen-binding fragment to a given epitope by at least 90%, at least 80%, at least 70%, at least 60%, or at least 50%.

As used herein, the term “affinity” refers to a measure of the strength of the binding of an individual epitope with one or more antigen-binding domains, e.g., of an immunoglobulin molecule. See, e.g., Harlow et al., Antibodies: A Laboratory Manual, (Cold Spring Harbor Laboratory Press, 2nd ed. 1988) at pages 27-28. As used herein, the term “avidity” refers to the overall stability of the complex between a population of antigen-binding domains and an antigen. See, e.g., Harlow at pages 29-34. Avidity is related to both the affinity of individual antigen-binding domains in the population with specific epitopes, and also the valencies of the immunoglobulins and the antigen. For example, the interaction between a bivalent monoclonal antibody and an antigen with a highly repeating epitope structure, such as a polymer, would be one of high avidity. An interaction between a bivalent monoclonal antibody with a receptor present at a high density on a cell surface would also be of high avidity.

Binding molecules, e.g., antibodies or fragments, variants, or derivatives thereof as disclosed herein can also be described or specified in terms of their cross-reactivity. As used herein, the term “cross-reactivity” refers to the ability of a binding molecule, e.g., an antibody or fragment, variant, or derivative thereof, specific for one antigen, to react with a second antigen; a measure of relatedness between two different antigenic substances. Thus, a binding molecule is cross reactive if it binds to an epitope other than the one that induced its formation. The cross-reactive epitope generally contains many of the same complementary structural features as the inducing epitope, and in some cases, can actually fit better than the original.

A binding molecule, e.g., an antibody or fragment, variant, or derivative thereof can also be described or specified in terms of their binding affinity to an antigen. For example, a binding molecule can bind to an antigen with a dissociation constant or K_{D} no greater than 5×10⁻²M, 10⁻²M, 5×10⁻³M, 10⁻³M, 5×10⁻⁴M, 10⁴ M, 5×10⁻⁵M, 10⁻⁵M, 5×10⁻⁶M, 10⁻⁶M, 5×10⁻⁷M, 10⁻⁷ M, 5×10⁻⁸M, 10⁻⁸M, 5×10⁻⁹M, 10⁻⁹M, 5×10⁻¹⁰ M, 10⁻¹⁰ M, 5×10⁻¹¹ M, 10⁻¹¹ M, 5×10⁻¹² M, 10⁻¹² M, 5×10⁻¹³M, 10⁻¹³M, 5×10⁻¹⁴M, 10⁻¹⁴ M, 5×10⁻¹⁵M, or 10⁻¹⁵M.

“Antigen-binding antibody fragments” including single-chain antibodies or other antigen-binding domains can exist alone or in combination with one or more of the following: hinge region, CH1, CH2, CH3, or CH4 domains, J-chain, or secretory component. Also included are antigen-binding fragments that can include any combination of variable region(s) with one or more of a hinge region, CH1, CH2, CH3, or CH4 domains, a J-chain, or a secretory component. Binding molecules, e.g., antibodies, or antigen-binding fragments thereof can be from any animal origin including birds and mammals. The antibodies can be human, murine, donkey, rabbit, goat, guinea pig, camel, llama, horse, or chicken antibodies. In another embodiment, the variable region can be condricthoid in origin (e.g., from sharks). As used herein, “human” antibodies include antibodies having the amino acid sequence of a human immunoglobulin and include antibodies isolated from human immunoglobulin libraries or from animals transgenic for one or more human immunoglobulins and can in some instances express endogenous immunoglobulins and some not, as described infra and, for example in, U.S. Pat. No. 5,939,598 by Kucherlapati et al. According to embodiments of the present disclosure, an IgM antibody, IgM-like antibody, or other IgM-derived binding molecule as provided herein can include an antigen-binding fragment of an antibody, e.g., a scFv fragment, so long as the IgM antibody, IgM-like antibody, or other IgM-derived binding molecule is able to form a multimer, e.g., a hexamer or a pentamer, and an IgA antibody, IgA-like antibody, or other IgA-derived binding molecule as provided herein can include an antigen-binding fragment of an antibody, e.g., a scFv fragment, so long as the IgA antibody, IgA-like antibody, or other IgA-derived binding molecule is able to form a multimer, e.g., a dimer and/or a tetramer. As used herein such a fragment comprises a “multimerizing fragment.”

As used herein, the term “heavy chain subunit” includes amino acid sequences derived from an immunoglobulin heavy chain, a binding molecule, e.g., an antibody, antibody-like, or antibody-derived molecule comprising a heavy chain subunit can include at least one of: a VH domain, a CH1 domain, a hinge (e.g., upper, middle, and/or lower hinge region) domain, a CH2 domain, a CH3 domain, a CH4 domain, or a variant or fragment thereof. For example, a binding molecule, e.g., an antibody, antibody-like, or antibody-derived molecule, or fragment, e.g., multimerizing fragment, variant, or derivative thereof can include without limitation, in addition to a VH domain: a CH1 domain; a CH1 domain, a hinge, and a CH2 domain; a CH1 domain and a CH3 domain; a CH1 domain, a hinge, and a CH3 domain; or a CH1 domain, a hinge domain, a CH2 domain, and a CH3 domain. In certain embodiments a binding molecule, e.g., an antibody, antibody-like, or antibody-derived molecule, or fragment, e.g., multimerizing fragment, variant, or derivative thereof can include, in addition to a VH domain, a CH3 domain and a CH4 domain; or a CH3 domain, a CH4 domain, and a J-chain. Further, a binding molecule, e.g., an antibody, antibody-like, or antibody-derived molecule, for use in the disclosure can lack certain constant region portions, e.g., all or part of a CH2 domain. It will be understood by one of ordinary skill in the art that these domains (e.g., the heavy chain subunit) can be modified such that they vary in amino acid sequence from the original immunoglobulin molecule. According to embodiments of the present disclosure, an IgM antibody, IgM-like antibody, or other IgM-derived binding molecule as provided herein comprises sufficient portions of an IgM heavy chain constant region to allow the IgM antibody, IgM-like antibody, or other IgM-derived binding molecule to form a multimer, e.g., a hexamer or a pentamer. As used herein such a fragment comprises a “multimerizing fragment.” According to embodiments of the present disclosure, an IgA antibody, IgA-like antibody, or other IgA-derived binding molecule as provided herein comprises sufficient portions of an IgA heavy chain constant region to allow the IgA antibody, IgA-like antibody, or other IgA-derived binding molecule to form a multimer, e.g., a dimer or a tetramer. As used herein such a fragment comprises a “multimerizing fragment.”

As used herein, the term “light chain subunit” includes amino acid sequences derived from an immunoglobulin light chain. The light chain subunit includes at least a VL, and can further include a CL (e.g., Cκ or Cλ) domain.

Binding molecules, e.g., antibodies, antibody-like molecules, antibody-derived molecules, antigen-binding fragments, variants, or derivatives thereof, or multimerizing fragments thereof can be described or specified in terms of the epitope(s) or portion(s) of a target, e.g., a target antigen that they recognize or specifically bind. The portion of a target antigen that specifically interacts with the antigen-binding domain of an antibody is an “epitope,” or an “antigenic determinant.” A target antigen can comprise a single epitope or at least two epitopes, and can include any number of epitopes, depending on the size, conformation, and type of antigen.

As used herein, the term “hinge region” includes the portion of a heavy chain molecule that joins the CH1 domain to the CH2 domain in IgG, IgA, and IgD heavy chains, and provides flexibility to the molecule.

As used herein the term “disulfide bond” includes the covalent bond formed between two sulfur atoms, e.g., in cysteine residues of a polypeptide. The amino acid cysteine comprises a thiol group that can form a disulfide bond or bridge with a second thiol group. Disulfide bonds can be “intra-chain,” i.e., linking to cysteine residues in a single polypeptide or polypeptide subunit, or can be “inter-chain,” i.e., linking two separate polypeptide subunits, e.g., an antibody heavy chain and an antibody light chain, to antibody heavy chains, or an IgM or IgA antibody heavy chain constant region and a J-chain.

As used herein, the term “chimeric antibody” refers to an antibody in which the immunoreactive region or site is obtained or derived from a first species and the constant region (which can be intact, partial, or modified) is obtained from a second species. In some embodiments the target binding region or site will be from a non-human source (e.g., mouse or primate) and the constant region is human.

The terms “multispecific antibody” or “bispecific antibody” refer to an antibody, antibody-like, or antibody-derived molecule that has antigen-binding domains for two or more different epitopes within a single antibody molecule. Other binding molecules in addition to the canonical antibody structure can be constructed with two binding specificities. Epitope binding by bispecific or multispecific antibodies can be simultaneous or sequential. Triomas and hybrid hybridomas are two examples of cell lines that can secrete bispecific antibodies. Bispecific antibodies can also be constructed by recombinant means. (Strohlein and Heiss, Future Oncol. 6:1387-94 (2010); Mabry and Snavely, IDrugs. 13:543-9 (2010)). A bispecific antibody can also be a diabody.

As used herein, the term “engineered antibody” refers to an antibody in which a variable domain, constant region, and/or J-chain is altered by at least partial replacement of one or more amino acids. In certain embodiments entire CDRs from an antibody of known specificity can be grafted into the framework regions of a heterologous antibody. Although alternate CDRs can be derived from an antibody of the same class or even subclass as the antibody from which the framework regions are derived, CDRs can also be derived from an antibody of different class, e.g., from an antibody from a different species. An engineered antibody in which one or more “donor” CDRs from a non-human antibody of known specificity are grafted into a human heavy or light chain framework region is referred to herein as a “humanized antibody.” In certain embodiments not all of the CDRs are replaced with the complete CDRs from the donor variable region and yet the antigen-binding capacity of the donor can still be transferred to the recipient variable domains. Given the explanations set forth in, e.g., U.S. Pat. Nos. 5,585,089, 5,693,761, 5,693,762, and 6,180,370, it will be well within the competence of those skilled in the art, either by carrying out routine experimentation or by trial-and-error testing, to obtain a functional engineered or humanized antibody.

As used herein the term “engineered” includes manipulation of nucleic acid or polypeptide molecules by synthetic means (e.g., by recombinant techniques, in vitro peptide synthesis, by enzymatic or chemical coupling of peptides, nucleic acids, or glycans, or some combination of these techniques).

As used herein, the terms “linked,” “fused” or “fusion” or other grammatical equivalents can be used interchangeably. These terms refer to the joining together of two more elements or components, by whatever means including chemical conjugation or recombinant means. An “in-frame fusion” refers to the joining of two or more polynucleotide open reading frames (ORFs) to form a continuous longer ORF, in a manner that maintains the translational reading frame of the original ORFs. Thus, a recombinant fusion protein is a single protein containing two or more segments that correspond to polypeptides encoded by the original ORFs (which segments are not normally so joined in nature.) Although the reading frame is thus made continuous throughout the fused segments, the segments can be physically or spatially separated by, for example, in-frame linker sequence. For example, polynucleotides encoding the CDRs of an immunoglobulin variable region can be fused, in-frame, but be separated by a polynucleotide encoding at least one immunoglobulin framework region or additional CDR regions, as long as the “fused” CDRs are co-translated as part of a continuous polypeptide.

In the context of polypeptides, a “linear sequence” or a “sequence” is an order of amino acids in a polypeptide in an amino to carboxyl terminal direction in which amino acids that neighbor each other in the sequence are contiguous in the primary structure of the polypeptide. A portion of a polypeptide that is “amino-terminal” or “N-terminal” to another portion of a polypeptide is that portion that comes earlier in the sequential polypeptide chain. Similarly, a portion of a polypeptide that is “carboxy-terminal” or “C-terminal” to another portion of a polypeptide is that portion that comes later in the sequential polypeptide chain. For example, in a typical antibody, the variable domain is “N-terminal” to the constant region, and the constant region is “C-terminal” to the variable domain.

The term “expression” as used herein refers to a process by which a gene produces a biochemical, for example, a polypeptide. The process includes any manifestation of the functional presence of the gene within the cell including, without limitation, gene knockdown as well as both transient expression and stable expression. It includes without limitation transcription of the gene into RNA, e.g., messenger RNA (mRNA), and the translation of such mRNA into polypeptide(s). If the final desired product is a biochemical, expression includes the creation of that biochemical and any precursors. Expression of a gene produces a “gene product.” As used herein, a gene product can be either a nucleic acid, e.g., a messenger RNA produced by transcription of a gene, or a polypeptide that is translated from a transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like.

The terms “neutralizing” or “neutralize” as used herein refer to the ability of a therapeutic, e.g., a therapeutic antibody, to reduce and/or prevent viral infectivity. The term “infectivity” as used herein refers to the ability of the virus to do one or more of attach to cells, enter cells, release its nucleic acid, replicate its nucleic acid and synthesize viral proteins, and package its nucleic acid into new virions that can be released from the infected cell. A virus can be neutralized, e.g., by a therapeutic antibody, via the antibody's ability to specifically bind to the virion and inhibit its ability to attach to a host cell receptor, thereby preventing entry into the host cell.

The terms “potency” or “antiviral potency” used herein refer to the amount of a therapeutic agent, e.g., a multimeric binding molecule as provided by the disclosure, required to produce an effect, e.g., inhibition of binding of the SARS-CoV-2 spike protein to its receptor angiotensin-converting enzyme 2 (ACE2), measured, e.g., as a 50% effective concentration (EC₅₀), neutralization of SARS-CoV2 infectivity, measured, e.g., as a 50% effective concentration (EC₅₀), therapeutic protection of a subject infected with SARS-CoV-2, measured, e.g., as a 50% effective dose (ED₅₀), or prophylactic protection of a subject susceptible to SARS-CoV-2 infection, measured, e.g., as a 50% effective dose (ED₅₀). Other factors that can contribute to antiviral potency of an anti-SARS-CoV2 antibody include, without limitation, a reduced risk of testing COVID-positive, a reduced risk of needing hospitalization, a reduced hospitalization time, a reduced time in an intensive care unit (ICU), a reduced viral load in an organ or tissue, e.g., the lungs, and/or a reduced need for supplemental oxygen.

The phrase “structural protein” of a virus as used herein refer to a protein that is a component of a mature assembled viral particle and includes synthetic and/or naturally occurring variants. The four main structural proteins of the SARS-CoV-2 virus include spike (S), envelope (E), membrane (M), and nucleic capsid (N). See, e.g., Sadia and Basra M. A. R., Drug Dev. Res. DOI: 10.1002/ddr.21710 (2020).

The phrase “escape mutant” as used herein refers a variant of an initial strain of SARS-CoV-2 that arises following contact of the initial strain of SARS-CoV-2, or cells infected with the initial strain of SARS-CoV-2, with an antibody capable of neutralizing the initial strain of SARS-CoV-2, where the escape mutant is more resistant to neutralization by the antibody or is no longer capable of being neutralized by the antibody. SARS-CoV-2 escape mutants can include one or more mutations, such as an amino acid substitutions, additions, or deletions, typically in the spike (S) protein, and typically in the receptor binding domain (RBD) of the S protein.

Terms such as “treating” or “treatment” or “to treat” or “alleviating” or “to alleviate” refer to therapeutic measures that cure, slow down, lessen symptoms of, lessen the severity of symptoms of, and/or halt or slow the progression of an existing diagnosed pathologic condition or disorder. Terms such as “prevent,” “prevention,” “avoid,” “deterrence,” “prophylactic,” and the like refer to prophylactic or preventative measures that can prevent the development of, or can reduce the symptoms of, a targeted pathologic condition or disorder in a subject who has not yet contracted the targeted pathologic condition or disorder. The targeted pathologic condition or disorder can be, for example, COVID-19. Thus, “those in need of treatment” can include those already infected with SARS-CoV-2 as well as those who wish to prevent infection, or reduce or alleviate COVID-19 symptoms should they become infected.

The terms “protect,” “protection,” “protective,” and other related terms, as used herein, refer to the ability of a therapeutic or prophylactic agent to confer a desirable effect on a subject diagnosed with or susceptible to an infectious disease such as COVID-19. Protection can include, for example, alleviation of or a reduction in COVID-19 symptoms in a subject infected with SARS-CoV-2, such that, for example, the subject does not need to be hospitalized or put on a ventilator. Protection can also include, for example, preventing healthcare workers, family members, or other contacts of COVID-19 patients from becoming infected with SARS-CoV-2, or if they do become infected, reducing the symptoms of COVID-19. As it applies to a therapeutic or prophylactic animal model, “protection” can include a lower 50% effective dose (ED₅₀) among a group of animal subjects challenged with the therapeutic agent either before or after challenge with SARS-CoV-2. Data points that can be used to measure ED₅₀ vary, e.g., with the animal model or the amount of SARS-CoV-2 used to challenge the animal subjects. Data points can include, e.g., measurement of the virus titer in the lungs of the animals, quantitative measurement of viral RNA present in infected subjects, weight loss, death, or disease symptoms such as fever or difficulty breathing.

The terms “antibody-dependent enhancement” and “ADE” refer to the situation where the binding of an antibody or related binding molecule can increase infectivity of an infectious virus, including coronaviruses. See, e.g., Wen, J., et al., Int. J. Infect. Dis. 100:483-489 (2020).

As used herein the terms “serum half-life” or “plasma half-life” refer to the time it takes (e.g., in minutes, hours, or days) following administration for the serum or plasma concentration of a drug, e.g., a binding molecule such as an antibody, antibody-like, or antibody-derived molecule or fragment, e.g., multimerizing fragment thereof as described herein, to be reduced by 50%. Two half-lives can be described: the alpha half-life, α half-life, or t_{1/2}α, which is the rate of decline in plasma concentrations due to the process of drug redistribution from the central compartment, e.g., the blood in the case of intravenous delivery, to a peripheral compartment (e.g., a tissue or organ), and the beta half-life, β half-life, or t_{1/2}β which is the rate of decline due to the processes of excretion or metabolism.

As used herein the term “area under the plasma drug concentration-time curve” or “AUC” reflects the actual body exposure to drug after administration of a dose of the drug and is expressed in mg*h/L. This area under the curve can be measured, e.g., from time 0 (t₀) to infinity (∞) and is dependent on the rate of elimination of the drug from the body and the dose administered.

As used herein, the term “mean residence time” or “MRT” refers to the average length of time the drug remains in the body.

By “subject” or “individual” or “animal” or “patient” is meant any subject. In certain embodiments the subject is a mammalian subject, for whom diagnosis, prognosis, or therapy is desired. Mammalian subjects include humans, domestic animals, farm animals, and zoo, sports, or pet animals such as dogs, cats, guinea pigs, rabbits, rats, mice, horses, swine, cows, bears, and so on.

As used herein, as the term “a subject that would benefit from therapy” refers to a subset of subjects, from amongst all prospective subjects, which would benefit from administration of a given therapeutic agent, e.g., a binding molecule such as an antibody, comprising one or more antigen-binding domains. Such binding molecules, e.g., antibodies, can be used, e.g., for a diagnostic procedure and/or for treatment or prevention of a disease.

### SARS-CoV-2 Binding Molecules

This disclosure provides a multimeric binding molecule comprising two to six bivalent binding units or variants or fragments thereof, where each binding unit comprises two IgM or IgA heavy chain constant regions or multimerizing fragments or variants thereof, each associated with an antigen binding domain, where three to twelve of the antigen binding domains are identical and specifically bind to the SARS-CoV-2 spike (S) protein receptor binding domain (RBD). In some embodiments, each identical immunoglobulin antigen binding domain comprises a heavy chain variable region (VH) and a light chain variable region (VL) comprising six immunoglobulin complementarity determining regions HCDR1, HCDR2, HCDR3, LCDR1, LCDR2, and LCDR3, wherein the HCDR1, HCDR2, HCDR3, LCDR1, LCDR2, and LCDR3, comprise, respectively, the amino acid sequences SEQ ID NO: 15, SEQ ID NO: 16, SEQ ID NO: 17, SEQ ID NO 19, SEQ ID NO. 20, and SEQ ID NO: 21; SEQ ID NO: 23, SEQ ID NO: 24, SEQ ID NO: 25, SEQ ID NO 27, SEQ ID NO. 28, and SEQ ID NO: 29; SEQ ID NO: 31, SEQ ID NO: 32, SEQ ID NO: 33, SEQ ID NO 35, SEQ ID NO. 36, and SEQ ID NO: 37; SEQ ID NO: 39, SEQ ID NO: 40, SEQ ID NO: 41, SEQ ID NO 43, SEQ ID NO. 44, and SEQ ID NO: 45; SEQ ID NO: 47, SEQ ID NO: 48, SEQ ID NO: 49, SEQ ID NO 51, SEQ ID NO. 52, and SEQ ID NO: 53; or SEQ ID NO: 55, SEQ ID NO: 56, SEQ ID NO: 57, SEQ ID NO 59, SEQ ID NO. 60, and SEQ ID NO: 61; where the CDR regions are defined according to Kabat. In some embodiments, each identical immunoglobulin antigen binding domain comprises a heavy chain variable region (VH) and a light chain variable region (VL) comprising six immunoglobulin complementarity determining regions HCDR1, HCDR2, HCDR3, LCDR1, LCDR2, and LCDR3, wherein the HCDR1, HCDR2, HCDR3, LCDR1, LCDR2, and LCDR3, comprise, respectively, the amino acid sequences SEQ ID NO: 62, SEQ ID NO: 63, SEQ ID NO: 64, SEQ ID NO: 65, SEQ ID NO: 66, and SEQ ID NO: 67; SEQ ID NO: 68, SEQ ID NO: 69, SEQ ID NO: 70, SEQ ID NO: 71, SEQ ID NO: 72, and SEQ ID NO: 73; SEQ ID NO: 74, SEQ ID NO: 75, SEQ ID NO: 76, SEQ ID NO: 77, SEQ ID NO: 78, and SEQ ID NO: 79; SEQ ID NO: 80, SEQ ID NO: 81, SEQ ID NO: 82, SEQ ID NO: 83, SEQ ID NO: 84, and SEQ ID NO: 85; SEQ ID NO: 86, SEQ ID NO: 87, SEQ ID NO: 88, SEQ ID NO: 89, SEQ ID NO: 90, and SEQ ID NO: 91; or SEQ ID NO: 92, SEQ ID NO: 93, SEQ ID NO: 94, SEQ ID NO: 95, SEQ ID NO: 96, and SEQ ID NO: 97; where the CDRs are defined according to IMGT. In some embodiments, the multimeric binding molecule has greater antiviral potency against SARS-CoV-2 than a bivalent reference IgG antibody comprising two of the binding domains that specifically bind to the SARS-CoV-2 S protein RBD. In some embodiments, the bivalent reference IgG antibody comprises two identical antigen binding domains comprising the VH and VL amino acid sequences SEQ ID NO: 14 and SEQ ID NO: 18, SEQ ID NO: 22 and SEQ ID NO: 26, SEQ ID NO: 30 and SEQ ID NO: 34, SEQ ID NO: 38 and SEQ ID NO: 42, SEQ ID NO: 46 and SEQ ID NO: 50, or SEQ ID NO: 54 and SEQ ID NO: 58, respectively. In some embodiments, the provided binding molecules can be used to treat or prevent Coronavirus Disease 2019 (COVID-19).

In some embodiments, the VH and VL comprise the amino acid sequences SEQ ID NO: 14 and SEQ ID NO: 18, SEQ ID NO: 22 and SEQ ID NO: 26, SEQ ID NO: 30 and SEQ ID NO: 34, SEQ ID NO: 38 and SEQ ID NO: 42, SEQ ID NO: 46 and SEQ ID NO: 50, or SEQ ID NO: 54 and SEQ ID NO: 58, respectively.

In some embodiments the HCDR1, HCDR2, HCDR3, LCDR1, LCDR2, and LCDR3, comprise, respectively, the amino acid sequences SEQ ID NO: 15, SEQ ID NO: 16, SEQ ID NO: 17, SEQ ID NO 19, SEQ ID NO. 20, and SEQ ID NO: 21; or SEQ ID NO: 39, SEQ ID NO: 40, SEQ ID NO: 41, SEQ ID NO 43, SEQ ID NO. 44, and SEQ ID NO: 45; wherein the CDR regions are defined according to Kabat. In some embodiments the HCDR1, HCDR2, HCDR3, LCDR1, LCDR2, and LCDR3, comprise, respectively, the amino acid sequences SEQ ID NO: 62, SEQ ID NO: 63, SEQ ID NO: 64, SEQ ID NO: 65, SEQ ID NO: 66, and SEQ ID NO: 67; or SEQ ID NO: 80, SEQ ID NO: 81, SEQ ID NO: 82, SEQ ID NO: 83, SEQ ID NO: 84, and SEQ ID NO: 85; wherein the CDR regions are defined according to IMGT. In certain embodiments the VH and VL comprise the amino acid sequences SEQ ID NO: 14 and SEQ ID NO: 18 or SEQ ID NO: 38 and SEQ ID NO: 42, respectively.

In some embodiments, the VH and VL of the multimeric binding molecule comprise the amino acid sequences SEQ ID NO: 14 and SEQ ID NO: 18, respectively, and the VH and VL of the bivalent reference IgG antibody comprise the amino acid sequences SEQ ID NO: 14 and SEQ ID NO: 18, respectively.

In some embodiments, the VH and VL of the multimeric binding molecule comprise the amino acid sequences SEQ ID NO: 38 and SEQ ID NO: 42, respectively, and the VH and VL of the bivalent reference IgG antibody comprise the amino acid sequences SEQ ID NO: 38 and SEQ ID NO: 42, respectively.

In certain embodiments, the greater antiviral potency of the multimeric binding molecule relative to the reference IgG can be measured, e.g., as inhibition of binding of the SARS-CoV-2 spike protein to its receptor angiotensin-converting enzyme 2 (ACE2) at a lower 50% effective concentration (EC₅₀) than the bivalent reference IgG antibody, inhibition of binding of the SARS-CoV-2 spike protein to ACE2 under conditions where the bivalent reference IgG antibody cannot inhibit binding, neutralization of SARS-CoV-2 infectivity at a lower EC₅₀ than the bivalent reference IgG antibody, neutralization of SARS-CoV-2 infectivity under conditions where the bivalent reference IgG antibody cannot neutralize SARS-CoV-2 infectivity, protection in a therapeutic animal model at a lower 50% effective dose (ED₅₀) than the bivalent IgG antibody, protection in the therapeutic animal model under conditions where the bivalent reference IgG antibody cannot protect, protection in a prophylactic animal model at a lower ED₅₀ than the bivalent IgG antibody, protection in the prophylactic animal model under conditions where the bivalent reference IgG antibody cannot protect, or any combination thereof.

In some embodiments, the provided multimeric binding molecule can neutralize infectivity SARS-CoV-2 at a lower EC₅₀ than the bivalent reference IgG antibody or can neutralize infectivity of SARS-CoV-2 under conditions where the bivalent reference IgG antibody cannot neutralize. In certain embodiments, the EC₅₀ is at least two-fold, at least five-fold, at least ten-fold, at least fifty-fold, at least 100-fold, at least 500-fold, or at least 1000-fold, or at least 10,000-fold lower than the EC₅₀ of the bivalent IgG antibody. The EC₅₀ can be measured either as mass per volume, e.g., μg/ml, or as the number of molecules present, e.g., moles/liter. In certain embodiments, the conditions where the bivalent reference IgG antibody cannot neutralize comprises neutralization of an antibody-resistant variant of SARS-CoV-2. In certain embodiments, the antibody resistant variant of SARS-CoV-2 comprises an “escape mutant” of a SARS-CoV-2 virus that arose following contact with the bivalent reference IgG antibody. By “SARS-CoV-2 virus that arose following contact with the bivalent reference IgG antibody” is meant a variant virus that arises in response to selective pressure from the bivalent reference IgG antibody. For example, an escape mutant can arise during an in vitro neutralization assay in which virus are contacted with the bivalent reference IgG antibody and then used to infect ACE2-expressing host cells, or during in in vivo infection of a subject anima, where the subject animal is administered the bivalent reference IgG antibody either prior to or subsequent to the virus infection. During viral replication in the host cells or subject animal mutations may arise that confer resistance to the bivalent reference IgG antibody.

In some embodiments the provided multimeric binding molecule can confer protection against SARS-CoV-2 infection in a therapeutic or prophylactic animal model at a lower 50% effective dose (ED₅₀) than the bivalent reference IgG antibody, or wherein the binding molecule can confer protection against SARS-CoV-2 infection in a therapeutic or prophylactic animal model under conditions where the bivalent reference IgG antibody cannot protect. As used herein, measurements of “protection” against SARS-CoV-2 infection in an animal model can include a reduced SARS-CoV-2 viral load in the subject animals, e.g., in the animals' lungs, survival of the subject animals from a lethal SARS-CoV-2 infection, and/or a reduction on symptoms typical of SARS-CoV-2 infection in the animal model, e.g., weight loss, fever, difficulty breathing, or neurological symptoms. The ED₅₀ can be measured either as mass per volume, e.g., μg/ml, or as the number of molecules present, e.g., moles/liter. In certain embodiments, the conditions where the bivalent reference IgG antibody cannot protect comprises a virus challenge with an antibody-resistant variant of SARS-CoV-2. In certain embodiments, the antibody resistant variant of SARS-CoV-2 comprises an “escape mutant” of a SARS-CoV-2 virus that arose following contact with the bivalent reference IgG antibody. In certain embodiments, the antibody resistant variant of SARS-CoV-2 comprises an “escape mutant” of a SARS-CoV-2 virus that arose following contact with the bivalent reference IgG antibody.

In some embodiments the multimeric binding molecule reduces, inhibits, or blocks the SARS-CoV-2 S protein from binding to ACE2 at a lower EC₅₀ than the bivalent reference IgG antibody or reduces, inhibits, or blocks the SARS-CoV-2 S protein from binding to ACE2 under conditions where the bivalent reference IgG antibody cannot reduce, inhibit, or block the SARS-CoV-2 S protein from binding to ACE2. In certain embodiments the ACE2 is human ACE2. In certain embodiments, ACE2 is expressed on the surface of a cell, e.g., a cultured host cell, e.g., a Vero cell, or a cell in a susceptible subject, e.g., a human subject. In some embodiments, the binding molecule inhibits SARS-CoV-2 binding to its receptor, e.g., ACE2, at a lower 50% effective concentration (EC₅₀) than the bivalent reference IgG antibody. In some embodiments, the EC₅₀ is at least two-fold, at least three-fold, at least four-fold, at least five-fold, at least ten-fold, at least twenty-fold, at least thirty-fold, at least forty-fold, or at least fifty-fold lower than the EC₅₀ of the bivalent reference IgG antibody.

The ability of an antibody to neutralize SARS-CoV-2 can readily be determined by one of skill in the art, such as by measuring infectivity in vitro using a viral or pseudoviral infectivity assay, such as an assay adapted from Richman et al. (PNAS, 2003, 100(7): 4144-4149) or described in Yuan et al., Science 10.1126/science.abb7269; (2020) or Muruato et al. (2020, Nat Comm. 11(1):4059. doi: 10.1038/s41467-020-17892-0).

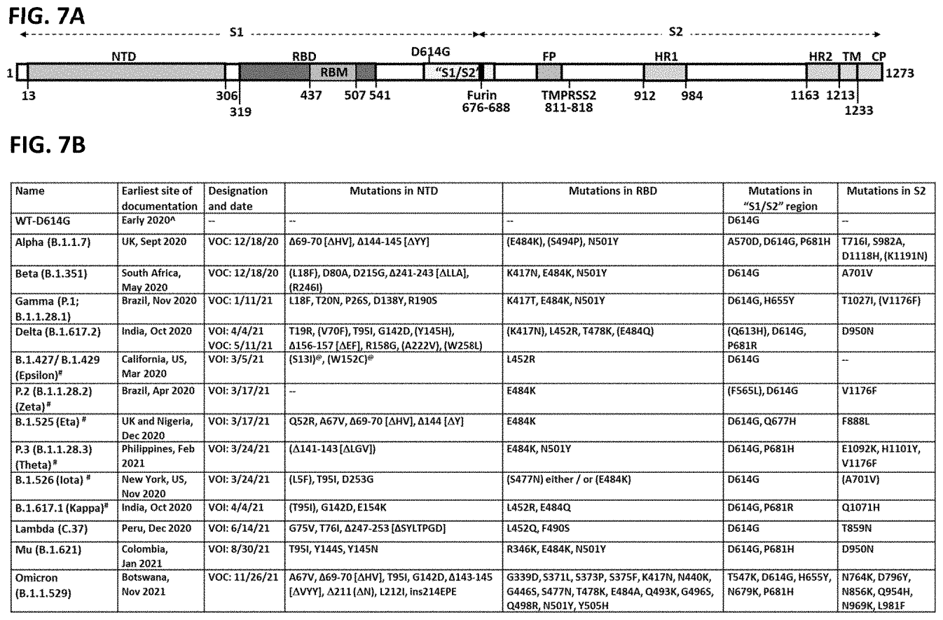

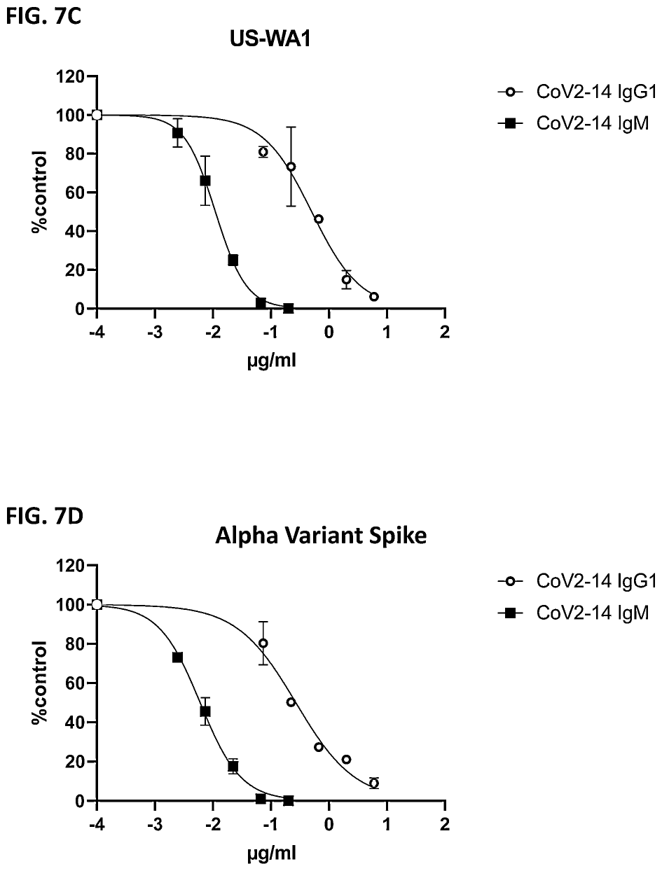

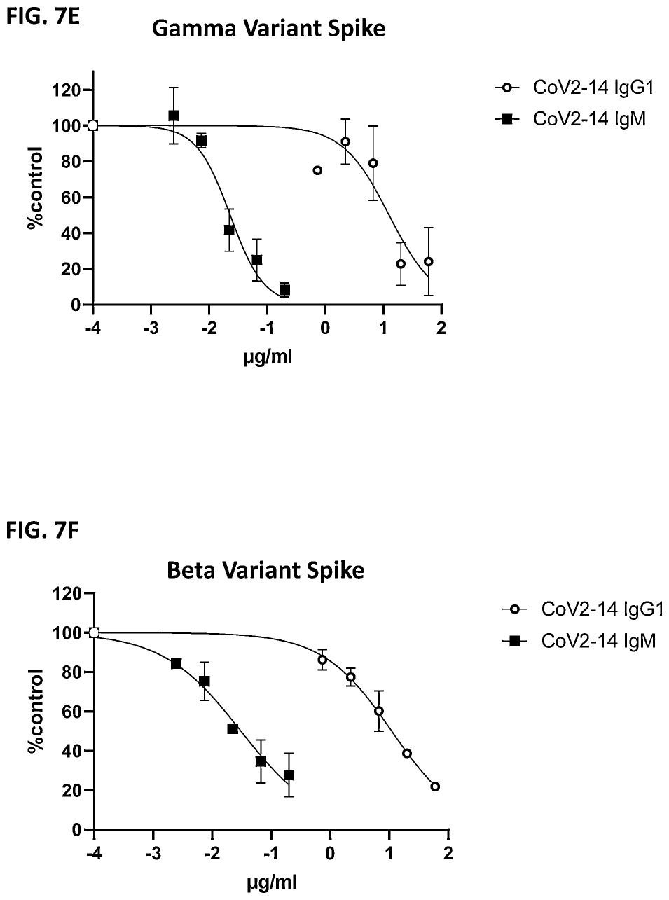

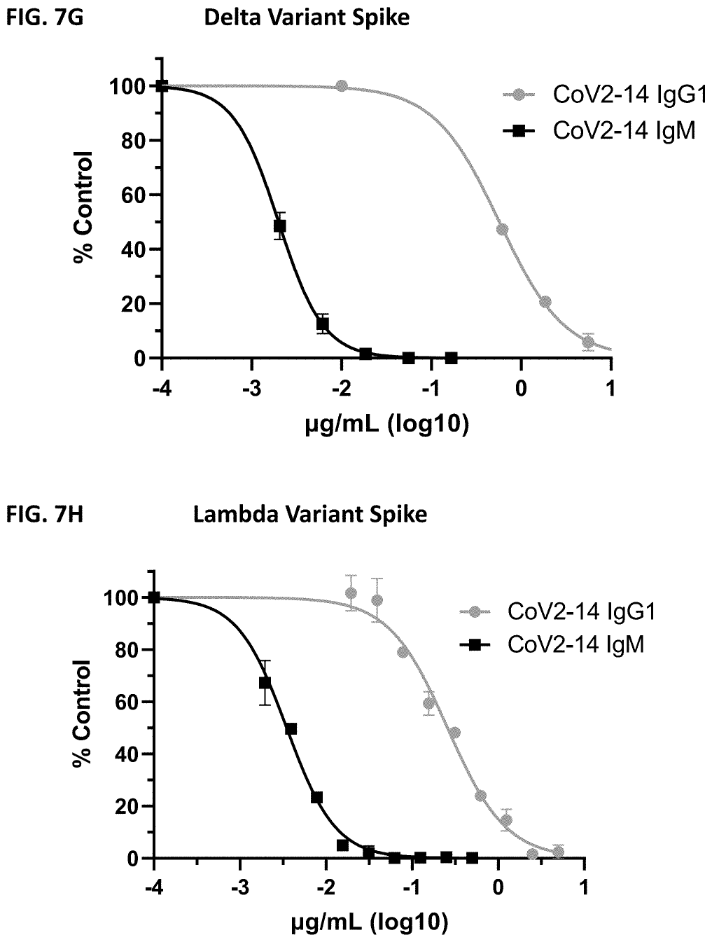

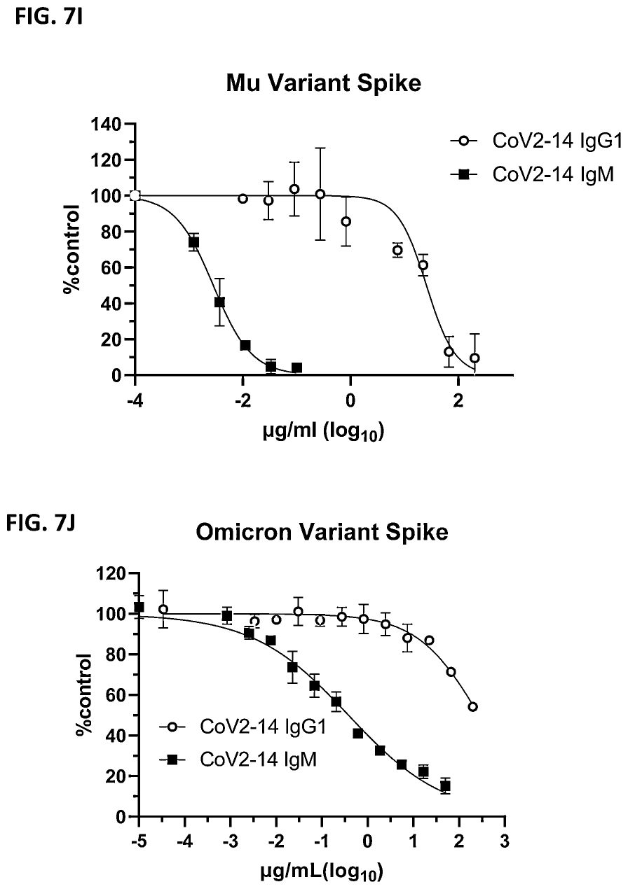

A reference SARS-CoV-2 S protein (UniProtKB—P0DTC2 (SPIKE_SARS2)) is presented herein as SEQ ID NO: 102. Myriad variant S proteins have been sequenced and are available in the literature but share the common structure of SEQ ID NO: 102. Certain Variants of Concern (VOC) and Variants of Interest (VOI) as identified by the World Health Organization are listed, along with their mutations, in FIGS. 7A and 7B. The spike protein is a single-pass type I membrane protein. The signal peptide of the SARS-CoV-2 S protein corresponds to amino acid 1 to amino acid 12 of SEQ ID NO: 102. The extracellular portion of the SARS-CoV-2 S protein corresponds to amino acids 13 to 1213 of SEQ ID NO: 2. The transmembrane domain of the SARS-CoV-2 S protein corresponds to amino acids 1214 to 1233 of SEQ ID NO: 2. The cytoplasmic domain of the SARS-CoV-2 S protein corresponds to amino acids 1234 to 1273 of SEQ ID NO: 102. The S protein RBD corresponds to amino acids 319 to 541 of SEQ ID NO: 102, underlined below (Yan, R. et al., Science 367:1444-1448 (2020)). As persons of ordinary skill in the art will recognize, the amino acid sequences SARS-CoV-2 S proteins, including the RBDs amino acid sequences of various SARS-CoV-2 S proteins, present in the environment have mutated to include amino acid substitutions, amino acid deletions, and amino acid insertions. See, e.g., FIG. 7A. Thus, a given SARS-CoV-2 S protein region or domain, e.g., an RBD that “corresponds” to amino acids 319 to 541 of SEQ ID NO: 102 may not be identical to amino acids 319 to 541 of SEQ ID NO: 102. The furin cleavage site between the 51 and S2 subunits is between amino acids 685 and 686, and is indicated by a vertical line (Hoffmann, M. et al., Cell 181:271-280 (2020)). The person of ordinary skill in the art will understand that the amino acid coordinates of the various regions and domains of the SARS-CoV-2 spike protein can be interpreted slightly differently by different investigators and different laboratories, and thus should be considered approximate.

```
SEQ ID NO: 102: SARS-CoV-2 Spike Protein, UniProt: P0DTC2
                10 20 30 40 50
MFVFLVLLPL VSSQCVNLTT RTQLPPAYTN SFTRGVYYPD KVFRSSVLHS
                60 70 80 90 100
TQDLFLPFFS NVTWFHAIHV SGTNGTKRFD NPVLPFNDGV YFASTEKSNI
              110 120 130 140 150
IRGWIFGTTL DSKTQSLLIV NNATNVVIKV CEFQFCNDPF LGVYYHKNNK
              160 170 180 190 200
SWMESEFRVY SSANNCTFEY VSQPFLMDLE GKQGNFKNLR EFVFKNIDGY
              210 220 230 240 250
FKIYSKHTPI NLVRDLPQGF SALEPLVDLP IGINITRFQT LLALHRSYLT
              260 270 280 290 300
PGDSSSGWTA GAAAYYVGYL QPRTFLLKYN ENGTITDAVD CALDPLSETK
              310 320 330 340 350
CTLKSFTVEK GIYQTSNFRV QPTESIVRFP NITNLCPFGE VFNATRFASV
              360 370 380 390 400
YAWNRKRISN CVADYSVLYN SASFSTFKCY GVSPTKLNDL CFTNVYADSF
              410 420 430 440 450
VIRGDEVRQI APGQTGKIAD YNYKLPDDFT GCVIAWNSNN LDSKVGGNYN
              460 470 480 490 500
YLYRLFRKSN LKPFERDIST EIYQAGSTPC NGVEGFNCYF PLQSYGFQPT
              510 520 530 540 550
NGVGYQPYRV VVLSFELLHA PATVCGPKKS TNLVKNKCVN FNFNGLTGTG
              560 570 580 590 600
VLTESNKKFL PFQQFGRDIA DTTDAVRDPQ TLEILDITPC SFGGVSVITP
              610 620 630 640 650
GTNTSNQVAV LYQDVNCTEV PVAIHADQLT PTWRVYSTGS NVFQTRAGCL
              660 670 680 690 700

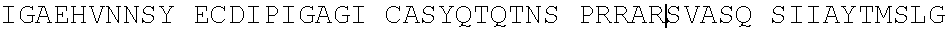

              710 720 730 740 750
AENSVAYSNN SIAIPTNFTI SVTTEILPVS MTKTSVDCTM YICGDSTECS
              760 770 780 790 800
NLLLQYGSFC TQLNRALTGI AVEQDKNTQE VFAQVKQIYK TPPIKDFGGF
              810 820 830 840 850
NFSQILPDPS KPSKRSFIED LLFNKVTLAD AGFIKQYGDC LGDIAARDLI
              860 870 880 890 900
CAQKFNGLTV LPPLLTDEMI AQYTSALLAG TITSGWTFGA GAALQIPFAM
              910 920 930 940 950
QMAYRFNGIG VTQNVLYENQ KLIANQFNSA IGKIQDSLSS TASALGKLQD
              960 970 980 990 1000
VVNQNAQALN TLVKQLSSNF GAISSVLNDI LSRLDKVEAE VQIDRLITGR
            1010 1020 1030 1040 1050
LQSLQTYVTQ QLIRAAEIRA SANLAATKMS ECVLGQSKRV DFCGKGYHLM
            1060 1070 1080 1090 1100
SFPQSAPHGV VFLHVTYVPA QEKNFTTAPA ICHDGKAHFP REGVFVSNGT
            1110 1120 1130 1140 1150
HWFVTQRNFY EPQIITTDNT FVSGNCDVVI GIVNNTVYDP LQPELDSFKE
            1160 1170 1180 1190 1200
ELDKYFKNHT SPDVDLGDIS GINASVVNIQ KEIDRLNEVA KNLNESLIDL
            1210 1220 1230 1240 1250
QELGKYEQYI KWPWYIWLGF IAGLIAIVMV TIMLCCMTSC CSCLKGCCSC
            1260 1270
GSCCKFDEDD SEPVLKGVKL HYT
```

Human coronaviruses are known to develop one or more mutations, particularly in the receptor binding domain (RBD) of the spike (S) protein over time that may alter the behavior of the virus. The variants are identified, for example, by nomenclature referred to as “pango” lineages (Rambaut, A., et al., Nature Microbiol. 5:1403-1407 (2020)), and “Variants of Interest” (VOI) or “Variants of Concern” (VOC) have been assigned Greek letter nomenclature by the World Health Organization (who.int/en/activities/tracking-SARS-CoV-2-variants/ (last visited Dec. 14, 2021). Variants of clinical relevance are cataloged by the Centers for Disease Control, e.g., at cdc.gov/coronavirus/2019-ncov/variants/variant-info.html (last visited Dec. 14, 2021). The CDC catalog is updated regularly. Those of skill in the art will understand that these lineages do not correspond to exact sequences and are generally characterized by the amino acid variations noted below, particularly in the RBD region, but may be mixtures and may include additional or fewer amino acid deletions, insertions, and/or substitutions relative to SEQ ID NO: 102. Key variants, along with their mutations corresponding to SEQ ID NO: 2, are listed in FIG. 7B.

For example, the pango lineage B.1.1.7 or WHO “Alpha” variant first identified in the UK includes an RBD substitution of tyrosine (Y) for asparagine (N) at a position corresponding to amino acid 501 (N501Y) in SEQ ID NO: 102, and can include additional spike protein alterations such as amino acid deletions at positions corresponding to amino acids 69, 70, 144, and 145 of SEQ ID NO: 102, and amino acid substitutions A570D, D614G, P681H, T716I, S982A, and D1118H corresponding to the indicated positions in SEQ ID NO: 102. Optional substitutions in subvariants can include, e.g., E484K, S494P, and K1191N (all positions corresponding to SEQ ID NO: 102). By “an amino acid corresponding to amino acid 501 in SEQ ID NO: 102 (and other spike protein mutations described herein) is meant the amino acid in the sequence of any given SARS-CoV-2 spike protein, which is homologous to N501 in SEQ ID NO: 102. Variant viruses carrying the “N501Y” mutation, including the Alpha variant, have been shown to be more highly transmissible than the non-variant virus. See Leung, K., et al., Euro Surveill. 26:2002106. doi: 10.2807/1560-7917.ES.2020.26.1.2002106 (2021).

The pango lineage B.1.351 or WHO “Beta” variant first identified in South Africa includes K417N, E484K and N501Y RBD substitutions corresponding to the indicated positions in SEQ ID NO: 102, and can include additional spike protein substitutions such as D80A, D215G, D614G, and A701V and amino acid deletions corresponding to amino acids 241-243 (all positions corresponding to SEQ ID NO: 102). Optional substitutions in subvariants can include, e.g., L18F and R246I (all positions corresponding to SEQ ID NO: 102). The Beta variant is believed to be more highly transmissible. See Wibmer, C K et al., Nature Med.:27:622-625, doi: 10.1038/s41591-021-01285-x (2021).

The pango lineage P.1 or WHO “Gamma” variant first identified in Brazil includes K417T, E484K, and N501Y RBD substitutions corresponding to the indicated positions in SEQ ID NO: 102, and can include additional spike protein substitutions such as L18F, T20N, P26S, D138Y, R1905, D614G, H655Y, and T10271 (all positions corresponding to SEQ ID NO: 102). Optional substitutions in subvariants can include, e.g., V1176F (corresponding to SEQ ID NO: 102). The Gamma variant is believed to be more highly transmissible. See Faria, Nuno R., et al., Virological (2021) (available at icpcovid.com, visited Feb. 19, 2021). Additionally, a SARS-CoV-2 variant with a D614G mutation is believed to have increased infectivity and transmissibility (Korber B., et al., Cell 182:812-827 (2020)).

The pango lineage B.1.525 or WHO “Eta” variant first identified in Nigeria includes an E484K RBD substitution corresponding to the indicated position in SEQ ID NO: 102, and can include additional spike protein alterations such as amino acid deletions at positions corresponding to amino acids 69, 70, and 144 of SEQ ID NO: 102, and spike protein substitutions Q52R, A67V, D614G, Q677H, F888L (all positions corresponding to SEQ ID NO: 102).

The pango lineage B.1.617.1 or WHO “Kappa” variant first identified in India includes L452R and E484Q RBD substitutions corresponding to the indicated positions in SEQ ID NO: 102, and can include spike protein substitutions such as G142D, E154K, D614G, P681R, and Q1071H, (all positions corresponding to SEQ ID NO: 102). Optional substitutions in subvariants can include, e.g., T95I (corresponding to SEQ ID NO: 102).

The pango lineage B.1.617.2. or WHO “Delta” variant first identified in India includes L452R and T478K RBD substitutions corresponding to the indicated positions in SEQ ID NO: 102, and can include additional spike protein alterations such as amino acid deletions at positions corresponding to amino acids 156 and 157 of SEQ ID NO: 102, and spike protein substitutions such as T19R, T95I, G142D, R158G, D614G, P681R, and D950N (all positions corresponding to SEQ ID NO: 102). Optional substitutions in subvariants can include, e.g., V70F, Y145H, A222V, W258L, K417N, E484Q, and Q613H (all positions corresponding to SEQ ID NO: 102). A particular subvariant of Delta called AY.4.3 carries, in addition to the standard Delta mutations, Y145H and A222V corresponding to SEQ ID NO: 102.

The pango lineage C.37 or WHO “Lambda” variant first identified in Peru includes L452Q and F490S RBD substitutions corresponding to the indicated positions in SEQ ID NO: 102, and can include additional spike protein alterations such as amino acid deletions at positions corresponding to amino acids 247-253 of SEQ ID NO: 102, and spike protein substitutions such as G75V, T76I, D614G, and T859N (all positions corresponding to SEQ ID NO: 102).

The pango lineage B.1.621 or WHO “Mu” variant first identified in Colombia includes R346K, E484K, and N501Y RBD substitutions corresponding to the indicated positions in SEQ ID NO: 102, and can include additional spike protein substitutions such as T95I, Y144S, Y145N, D614G, P681H, and D950N (all positions corresponding to SEQ ID NO: 102).

The pango lineage B.1.1.529 or WHO “Omicron” variant first identified in Botswana includes G339D, S371L, S373P, S375F, K417N, N440K, G446S, S477N, T478K, E484A, Q493K, G496S, Q498R, N501Y, and Y505H RBD substitutions corresponding to the indicated positions in SEQ ID NO: 102, and can include additional spike protein alterations such as amino acid deletions at positions corresponding to amino acids 69-70, 143-145, and 211 of SEQ ID NO: 102, an insertion of the amino acids EPE following position 214 in SEQ ID NO: 102, and can include additional spike protein substitutions such as A67V, T95I, G142D, L212I, T547K, D614G, H655Y, N679K, P681H, N764K, D796Y, N856K, Q954H, N969K, and L981F (all positions corresponding to SEQ ID NO: 102).

The pango lineage B.1.618 variant first identified in India includes an E484K RBD substitution corresponding to the indicated position in SEQ ID NO: 102, and can include additional spike protein alterations such as amino acid deletions at positions corresponding to amino acids 145 and 146 of SEQ ID NO: 102, and spike protein substitutions such as H49Y and D614G (all positions corresponding to SEQ ID NO: 102).

Of particular concern is the potential development of escape mutations that can reduce or prevent neutralization by a therapy, such as an antibody therapy. The multimeric binding molecules disclosed herein may be able to maintain the ability to bind and neutralize strains of SARS-CoV-2 that are escape mutants for the corresponding IgG antibody. For example, CoV2-14 IgM can neutralize a SARS-CoV-2 virus isolate comprising an E484A escape mutation isolated following contact with CoV2-14 IgG, where the IgG antibody does not. Additionally, the multimeric binding molecules disclosed herein may be less prone to generating escape mutants.

Additional prevalent escape mutations to published SARS-CoV-2 neutralizing antibodies include, without limitation, N439K, S477N and N501Y, which are three prevalent RBD mutations in circulation and are associated with resistance to several neutralizing mAbs (Thomson, E. C., et al. Cell 184(5):1171-1187.e20. doi: 10.1016/j.cell.2021.01.037 (2021); Liu, Z., et al. Cell Host Microb. 29:477-488 doi: 10.1016/j.chom.2021.01.014 (2021); Weisblum, Y., et al., eLife 9:e61312 doi: 10.7554/eLife.61312 (Oct. 28, 2020)). Other RBD mutations are associated with resistance to three approved mAbs, Bamlanivimab (E484K, F490S, Q493R, S494P), Casirivimab (REGN-10933, K417E, Y453F, L455F, G476S, F486V, Q493K) and Imdevimab (REGN-10987, K444Q, V445A, G446V) (Fact Sheet for Health Care Providers Emergency Use Authorization (EUA) of Bamlanivimab, (2020); Fact Sheet for Health Care Providers Emergency Use Authorization (EUA) Of Casirivimab and Imdevimab, (2020), both available at fda.gov (visited Jan. 25, 2021)). Additional escape mutations in the SARS-CoV-2 RBD are reported in Greaney, A J., et al., Nature Comm. 12:4196 doi: 10.1038/s41467-021-24435-8 (2021).

Antibody-dependent enhancement (ADE) of diseases caused by human coronaviruses is a concern (Houser, K. V., et al., PLoS Pathog. 13: Article e1006565 (2017) (MERS-CoV); Weiss, R. C., and F. W. Scott Comp. Immunol. Microbiol. Infect. Dis 4:175-189 (1981) (feline infectious peritonitis virus); and Kam, Y. W., et al., Vaccine 25:729-740 (2007) (SARS-CoV)). It is believed that Fey receptors may mediate antibody dependent entry into cells (Kam et al., supra). Fcγ receptors do not bind IgA or IgM antibodies. Accordingly, the multimeric binding molecules disclosed herein can have a reduced risk of ADE than the reference IgG antibody. In some embodiments, the multimeric binding molecule cannot cause ADE.

In some embodiments, the multimeric binding molecules are dimeric and comprise two bivalent binding units or variants or fragments thereof. In some embodiments, the multimeric binding molecules are dimeric, comprise two bivalent binding units or variants or fragments thereof, and further comprise a J-chain or functional fragment or variant thereof as described herein. In some embodiments, the multimeric binding molecules are dimeric, comprise two bivalent binding units or variants or fragments thereof, and further comprise a J-chain or functional fragment or variant thereof as described herein, where each binding unit comprises two IgA heavy chain constant regions or multimerizing fragments or variants thereof.

In some embodiments, the multimeric binding molecules are tetrameric and comprise four bivalent binding units or variants or fragments thereof. In some embodiments, the multimeric binding molecules are tetrameric, comprise four bivalent binding units or variants or fragments thereof, and further comprise a J-chain or functional fragment or variant thereof as described herein. In some embodiments, the multimeric binding molecules are tetrameric, comprise four bivalent binding units or variants or fragments thereof, and further comprise a J-chain or functional fragment or variant thereof as described herein, where each binding unit comprises two IgA heavy chain constant regions or multimerizing fragments or variants thereof.

In some embodiments, the multimeric binding molecules are pentameric and comprise five bivalent binding units or variants or fragments thereof. In some embodiments, the multimeric binding molecules are pentameric and comprise five bivalent binding units or variants or fragments thereof, and further comprise a J-chain or functional fragment or variant thereof as described herein. In some embodiments, the multimeric binding molecules are pentameric and comprise five bivalent binding units or variants or fragments thereof, and further comprise a J-chain or functional fragment or variant thereof as described herein, where each binding unit comprises two IgM heavy chain constant regions or multimerizing fragments or variants thereof.

In some embodiments, the multimeric binding molecules are hexameric and comprise six bivalent binding units or variants or fragments thereof. In some embodiments, the multimeric binding molecules are hexameric and comprise six bivalent binding units or variants or fragments thereof, and where each binding unit comprises two IgM heavy chain constant regions or multimerizing fragments or variants thereof.

In certain embodiments, heavy chain constant regions in the provided binding molecules are each associated with a VH subunit of an antibody antigen-binding domain. The multimeric binding molecule discloses herein can comprise three to twelve binding domains that are SARS-CoV-2 binding domains. In some embodiments, the multimeric binding molecule, such as an IgA antibody, an IgA-like antibody, or an IgA-derived binding molecule comprises three to eight binding domains that specifically bind to SARS-CoV-2. In some embodiments, the multimeric binding molecule, such as an IgA antibody, an IgA-like antibody, or an IgA-derived binding molecule comprises four binding domains that specifically bind to SARS-CoV-2. In some embodiments, the multimeric binding molecule, such as an IgA antibody, an IgA-like antibody, or an IgA-derived binding molecule comprises eight binding domains that specifically bind to SARS-CoV-2. In some embodiments, the multimeric binding molecule, such as an IgM antibody, an IgM-like antibody, or an IgM-derived binding molecule comprises ten or twelve binding domains that specifically bind to SARS-CoV-2.

In certain embodiments, the provided multimeric binding molecule is multispecific, e.g., bispecific, trispecific, or tetraspecific, where two or more binding domains associated with the heavy chain constant regions of the binding molecule specifically bind to different targets. In certain embodiments, the binding domains of the multimeric binding molecule all specifically bind to SARS-CoV-2. In certain embodiments, the binding domains of the multimeric binding molecule are identical. In such cases, the multimeric binding molecule can still be bispecific, if, for example, a binding domain with a different specificity is part of a modified J-chain as described elsewhere herein. In certain embodiments, the binding domains are antibody-derived antigen-binding domains, e.g., a scFv associated with the heavy chain constant regions or a VH subunit of an antibody binding domain associated with the heavy chain constant regions.

In certain embodiments, each binding unit comprises two heavy chains each comprising a VH situated amino terminal to the heavy chain constant region, and two immunoglobulin light chains each comprising a light chain variable domain (VL) situated amino terminal to an immunoglobulin light chain constant region, e.g., a kappa or lambda constant region. The provided VH and VL combine to form an antigen-binding domain that specifically binds to the target. In certain embodiments each antigen-binding domain of each binding molecule binds to SARS-CoV-2. In certain embodiments, each antigen-binding domain of each binding molecule is identical.

In certain embodiments, the three to twelve identical binding domains of the multimeric binding molecule that specifically bind to the SARS-CoV-2 spike (S) protein receptor binding domain (RBD) are identical, where each identical immunoglobulin antigen binding domain comprises a heavy chain variable region (VH) and a light chain variable region (VL) comprising six immunoglobulin complementarity determining regions HCDR1, HCDR2, HCDR3, LCDR1, LCDR2, and LCDR3, wherein the HCDR1, HCDR2, HCDR3, LCDR1, LCDR2, and LCDR3, comprise, respectively, the amino acid sequences SEQ ID NO: 15, SEQ ID NO: 16, SEQ ID NO: 17, SEQ ID NO 19, SEQ ID NO. 20, and SEQ ID NO: 21; SEQ ID NO: 23, SEQ ID NO: 24, SEQ ID NO: 25, SEQ ID NO 27, SEQ ID NO. 28, and SEQ ID NO: 29; SEQ ID NO: 31, SEQ ID NO: 32, SEQ ID NO: 33, SEQ ID NO 35, SEQ ID NO. 36, and SEQ ID NO: 37; SEQ ID NO: 39, SEQ ID NO: 40, SEQ ID NO: 41, SEQ ID NO 43, SEQ ID NO. 44, and SEQ ID NO: 45; SEQ ID NO: 47, SEQ ID NO: 48, SEQ ID NO: 49, SEQ ID NO 51, SEQ ID NO. 52, and SEQ ID NO: 53; or SEQ ID NO: 55, SEQ ID NO: 56, SEQ ID NO: 57, SEQ ID NO 59, SEQ ID NO. 60, and SEQ ID NO: 61, where the CDR regions are defined according to Kabat. In certain embodiments, the three to twelve identical binding domains of the multimeric binding molecule that specifically bind to the SARS-CoV-2 spike (S) protein receptor binding domain (RBD) are identical, where each identical immunoglobulin antigen binding domain comprises a heavy chain variable region (VH) and a light chain variable region (VL) comprising six immunoglobulin complementarity determining regions HCDR1, HCDR2, HCDR3, LCDR1, LCDR2, and LCDR3, wherein the HCDR1, HCDR2, HCDR3, LCDR1, LCDR2, and LCDR3, comprise, respectively, the amino acid sequences SEQ ID NO: 62, SEQ ID NO: 63, SEQ ID NO: 64, SEQ ID NO: 65, SEQ ID NO: 66, and SEQ ID NO: 67; SEQ ID NO: 68, SEQ ID NO: 69, SEQ ID NO: 70, SEQ ID NO: 71, SEQ ID NO: 72, and SEQ ID NO: 73; SEQ ID NO: 74, SEQ ID NO: 75, SEQ ID NO: 76, SEQ ID NO: 77, SEQ ID NO: 78, and SEQ ID NO: 79; SEQ ID NO: 80, SEQ ID NO: 81, SEQ ID NO: 82, SEQ ID NO: 83, SEQ ID NO: 84, and SEQ ID NO: 85; SEQ ID NO: 86, SEQ ID NO: 87, SEQ ID NO: 88, SEQ ID NO: 89, SEQ ID NO: 90, and SEQ ID NO: 91; or SEQ ID NO: 92, SEQ ID NO: 93, SEQ ID NO: 94, SEQ ID NO: 95, SEQ ID NO: 96, and SEQ ID NO: 97; where the CDR regions are defined according to IMGT.

In some embodiments, the VH and VL comprise the amino acid sequences SEQ ID NO: 14 and SEQ ID NO: 18, SEQ ID NO: 22 and SEQ ID NO: 26, SEQ ID NO: 30 and SEQ ID NO: 34, SEQ ID NO: 38 and SEQ ID NO: 42, SEQ ID NO: 46 and SEQ ID NO: 50, or SEQ ID NO: 54 and SEQ ID NO: 58, respectively. In some embodiments, the HCDR1, HCDR2, HCDR3, LCDR1, LCDR2, and LCDR3, comprise, respectively, the amino acid sequences SEQ ID NO: 15, SEQ ID NO: 16, SEQ ID NO: 17, SEQ ID NO 19, SEQ ID NO. 20, and SEQ ID NO: 21, or SEQ ID NO: 39, SEQ ID NO: 40, SEQ ID NO: 41, SEQ ID NO 43, SEQ ID NO. 44, and SEQ ID NO: 45; where the CDR regions are defined according to Kabat. In some embodiments, the HCDR1, HCDR2, HCDR3, LCDR1, LCDR2, and LCDR3, comprise, respectively, the amino acid sequences SEQ ID NO: 62, SEQ ID NO: 63, SEQ ID NO: 64, SEQ ID NO: 65, SEQ ID NO: 66, and SEQ ID NO: 67; or SEQ ID NO: 80, SEQ ID NO: 81, SEQ ID NO: 82, SEQ ID NO: 83, SEQ ID NO: 84, and SEQ ID NO: 85; where the CDR regions are defined according to IMGT. In some embodiments, the VH and VL comprise the amino acid sequences SEQ ID NO: 14 and SEQ ID NO: 18 or SEQ ID NO: 38 and SEQ ID NO: 42, respectively.

### IgM Antibodies, IgM-Like Antibodies, Other IgM-Derived Binding Molecules

IgM is the first immunoglobulin produced by B cells in response to stimulation by antigen. Naturally-occurring IgM is naturally present at around 1.5 mg/ml in serum with a half-life of about 5 days. IgM is a pentameric or hexameric molecule and thus includes five or six binding units. An IgM binding unit typically includes two light and two heavy chains. While an IgG heavy chain constant region contains three heavy chain constant domains (CH1, CH2 and CH3), the heavy (μ) constant region of IgM additionally contains a fourth constant domain (CH4) and includes a C-terminal “tailpiece.” The human IgM constant region typically comprises the amino acid sequence SEQ ID NO: 1 (identical to, e.g., GenBank Accession Nos. pir∥S37768, CAA47708.1, and CAA47714.1, allele IGHM*03) or SEQ ID NO: 2 (identical to, e.g., GenBank Accession No. sp|P01871.4, allele IGHM*04). The human Cμ1 region ranges from about amino acid 5 to about amino acid 102 of SEQ ID NO: 1 or SEQ ID NO: 2; the human Cμ2 region ranges from about amino acid 114 to about amino acid 205 of SEQ ID NO: 1 or SEQ ID NO: 2, the human Cμ3 region ranges from about amino acid 224 to about amino acid 319 of SEQ ID NO: 1 or SEQ ID NO: 2, the Cp. 4 region ranges from about amino acid 329 to about amino acid 430 of SEQ ID NO: 1 or SEQ ID NO: 2, and the tailpiece ranges from about amino acid 431 to about amino acid 453 of SEQ ID NO: 1 or SEQ ID NO: 2.

Other forms and alleles of the human IgM constant region with minor sequence variations exist, including, without limitation, GenBank Accession Nos. CAB37838.1, and pir∥MHHU. The amino acid substitutions, insertions, and/or deletions at positions corresponding to SEQ ID NO: 1 or SEQ ID NO: 2 described and claimed elsewhere in this disclosure can likewise be incorporated into alternate human IgM sequences, as well as into IgM constant region amino acid sequences of other species.

Each IgM heavy chain constant region can be associated with a VH region. Exemplary VH regions that bind SARS-CoV-2 are described elsewhere herein.

Five IgM binding units can form a complex with an additional small polypeptide chain (the J-chain), or a functional fragment, variant, or derivative thereof, to form a pentameric IgM antibody or IgM-like antibody, as discussed elsewhere herein. The precursor form of the human J-chain is presented as SEQ ID NO: 6. The signal peptide extends from amino acid 1 to about amino acid 22 of SEQ ID NO: 6, and the mature human J-chain extends from about amino acid 23 to amino acid 159 of SEQ ID NO: 6. The mature human J-chain includes the amino acid sequence SEQ ID NO: 7.

Exemplary variant and modified J-chains are provided elsewhere herein. Without the J-chain, an IgM antibody or IgM-like antibody typically assembles into a hexamer, comprising up to twelve antigen-binding domains. With a J-chain, an IgM antibody or IgM-like antibody typically assembles into a pentamer, comprising up to ten antigen-binding domains, or more, if the J-chain is a modified J-chain comprising one or more heterologous polypeptides comprising additional antigen-binding domain(s). The assembly of five or six IgM binding units into a pentameric or hexameric IgM antibody or IgM-like antibody is thought to involve the Cμ4 and tailpiece domains. See, e.g., Braathen, R., et al., J. Biol. Chem. 277:42755-42762 (2002). Accordingly, a pentameric or hexameric IgM antibody provided in this disclosure typically includes at least the Cμ4 and tailpiece domains (also referred to herein collectively as Cμ4-tp). A “multimerizing fragment” of an IgM heavy chain constant region thus includes at least the Cμ4-tp domains. An IgM heavy chain constant region can additionally include a Cμ3 domain or a fragment thereof, a Cμ2 domain or a fragment thereof, a Cμ1 domain or a fragment thereof, and/or other IgM heavy chain domains. In certain embodiments, an IgM-derived binding molecule, e.g., an IgM antibody, IgM-like antibody, or other IgM-derived binding molecule as provided herein can include a complete IgM heavy (μ) chain constant domain, e.g., SEQ ID NO: 1 or SEQ ID NO: 2, or a variant, derivative, or analog thereof, e.g., as provided herein.

In certain embodiments, the disclosure provides a multimeric binding molecule, e.g., a pentameric or hexameric binding molecule, where the binding molecule includes ten or twelve IgM-derived heavy chains, and where the IgM-derived heavy chains comprise IgM heavy chain constant regions each associated with a binding domain that specifically binds to a target. In certain embodiments, the disclosure provides an IgM antibody, IgM-like antibody, or IgM-derived binding molecule that includes five or six bivalent binding units, where each binding unit includes two IgM or IgM-like heavy chain constant regions or multimerizing fragments or variants thereof, each associated with an antigen-binding domain or subunit thereof. In certain embodiments, the two IgM heavy chain constant regions included in each binding unit are human heavy chain constant regions. In some embodiments, the heavy chains are glycosylated. In some embodiments, the heavy chains can be mutated to affect glycosylation.

Where the IgM antibody, IgM-like antibody, or other IgM-derived binding molecule provided in this disclosure is pentameric, the IgM antibody, IgM-like antibody, or other IgM-derived binding molecule typically further includes a J-chain, or functional fragment or variant thereof. In certain embodiments, the J-chain is a modified J-chain or variant thereof that further comprises one or more heterologous moieties attached to the J-chain, as described elsewhere herein. In certain embodiments, the J-chain can be mutated to affect, e.g., enhance, the serum half-life of the IgM antibody, IgM-like antibody, or other IgM-derived binding molecule provided herein, as discussed elsewhere in this disclosure. In certain embodiments the J-chain can be mutated to affect glycosylation and/or serum half-life of the binding molecule, as discussed elsewhere in this disclosure.

An IgM heavy chain constant region can include one or more of a Cμ1 domain or fragment or variant thereof, a Cμ2 domain or fragment or variant thereof, a Cμ3 domain or fragment or variant thereof, a Cμ4 domain or fragment or variant thereof, and/or an IgM tailpiece, provided that the constant region can serve a desired function in the IgM antibody, IgM-like antibody, or other IgM-derived binding molecule, e.g., associate with second IgM constant region to form a binding unit with one, two, or more antigen-binding domain(s), and/or associate with other binding units (and in the case of a pentamer, a J-chain) to form a hexamer or a pentamer. In certain embodiments the two IgM heavy chain constant regions or fragments or variants thereof within an individual binding unit each comprise a Cμ4 domain or fragment or variant thereof, a tailpiece (tp) or fragment or variant thereof, or a combination of a Cμ4 domain and a TP or fragment or variant thereof. In certain embodiments the two IgM heavy chain constant regions or fragments or variants thereof within an individual binding unit each further comprise a Cμ3 domain or fragment or variant thereof, a Cμ2 domain or fragment or variant thereof, a Cμ1 domain or fragment or variant thereof, or any combination thereof.

In some embodiments, the binding units of the IgM antibody, IgM-like antibody, or other IgM-derived binding molecule each comprise two light chains. In some embodiments, the binding units of the IgM antibody, IgM-like antibody, or other IgM-derived binding molecule each comprise two fragments of light chains. In some embodiments, the light chains are kappa light chains. In some embodiments, the light chains are lambda light chains. In some embodiments, the light chains are hybrid kappa-lambda light chains. In some embodiments, each binding unit comprises two immunoglobulin light chains each comprising a VL situated amino terminal to an immunoglobulin light chain constant region.

### IgA Antibodies, IgA-Like Antibodies, Other IgA-Derived Binding Molecules

IgA plays a critical role in mucosal immunity and comprises about 15% of total immunoglobulin produced. IgA can be monomeric or multimeric, forming primarily dimeric molecules, but can also assemble as trimers, tetramers, and/or pentamers. See, e.g., de Sousa-Pereira, P., and J. M. Woof, Antibodies 8:57 (2019). An IgA binding unit typically includes two light and two heavy chains. IgA contains three heavy chain constant region domains (Cα1, Cα2 and Cα3), a hinge region between Cα1 and Cα2, and includes a C-terminal “tailpiece.” Human IgA has two subtypes, IgA1 and IgA2. The human IgA1 constant region typically includes the amino acid sequence SEQ ID NO: 3. The human Cα1 domain extends from about amino acid 6 to about amino acid 98 of SEQ ID NO: 3; the human IgA1 hinge region extends from about amino acid 102 to about amino acid 124 of SEQ ID NO: 3, the human Cα3 domain extends from about amino acid 228 to about amino acid 330 of SEQ ID NO: 3, and the tailpiece extends from about amino acid 331 to about amino acid 352 of SEQ ID NO: 3. The human IgA2 constant region typically includes the amino acid sequence SEQ ID NO: 4. The human Cα1 domain extends from about amino acid 6 to about amino acid 98 of SEQ ID NO: 4; the human IgA2 hinge region extends from about amino acid 102 to about amino acid 111 of SEQ ID NO: 4, the human Cα2 domain extends from about amino acid 113 to about amino acid 206 of SEQ ID NO: 4, the human Cα3 domain extends from about amino acid 215 to about amino acid 317 of SEQ ID NO: 4, and the tailpiece extends from about amino acid 318 to about amino acid 340 of SEQ ID NO: 4.

Two IgA binding units can form a complex with two additional polypeptide chains, the J-chain (e.g., the mature human J-chain of SEQ ID NO: 7) and the secretory component (precursor, SEQ ID NO: 5, mature: amino acids 19 to 603 of SEQ ID NO: 5) to form a secretory IgA (sIgA) antibody. The assembly of IgA binding units into a dimeric sIgA antibody is thought to involve the Cα3 and tailpiece domains (also referred to herein collectively as the Cα3-tp domain).

Accordingly, a dimeric sIgA antibody provided in this disclosure typically includes IgA constant regions that include at least the Cα3 and tailpiece domains. Four IgA binding units can likewise form a tetramer complex with a J-chain. A sIgA antibody can also form as a higher order multimer, e.g., a pentamer.

An IgA heavy chain constant region can additionally include a Cα2 domain or a fragment thereof, an IgA hinge region, a Cα1 domain or a fragment thereof, and/or other IgA heavy chain domains. In certain aspects, an IgA antibody or IgA-like binding molecule as provided herein can include a complete IgA heavy (a) chain constant domain (e.g., SEQ ID NO: 3 or SEQ ID NO: 4), or a variant, derivative, or analog thereof. In some embodiments, the IgA heavy chain constant regions or multimerizing fragments thereof are human IgA constant regions.

In some embodiments, each binding unit of an IgA antibody, IgA-like antibody, or other IgA-derived binding molecule comprises two light chains. In some embodiments, each binding unit of an IgA antibody, IgA-like antibody, or other IgA-derived binding molecule comprises two fragments of light chains. In some embodiments, the light chains are kappa light chains. In some embodiments, the light chains are lambda light chains. In some embodiments the light chains are hybrid kappa-lambda light chains. In some embodiments, each binding unit comprises two immunoglobulin light chains each comprising a VL situated amino terminal to an immunoglobulin light chain constant region.

### J-Chains and Functional Fragments or Variants Thereof

In certain embodiments, the multimeric binding molecule provided herein comprises a J-chain or functional fragment or variant thereof. In certain embodiments, the multimeric binding molecule provided herein is pentameric and comprises a J-chain or functional fragment or variant thereof. In certain embodiments, the multimeric binding molecule provided herein is a dimeric IgA molecule or a pentameric IgM molecule and comprises a J-chain or functional fragment or variant thereof. In some embodiments, the multimeric binding molecule can comprise a naturally occurring J-chain sequence, such as a mature human J-chain sequence (e.g., SEQ ID NO: 7). Alternatively, in some embodiments, the multimeric binding molecule can comprise a variant J-chain sequence, such as a variant sequence described herein with reduced glycosylation or reduced binding to one or more polymeric Ig receptors (e.g., pIgR, Fc alpha-mu receptor (FcαμR), or Fc mu receptor (FcμR)). See, e.g., U.S. Pat. No. 10,899,835, which is incorporated herein by reference in its entirety. In some embodiments, the multimeric binding molecule can comprise a functional fragment of a naturally occurring or variant J-chain. As persons of ordinary skill in the art will recognize, “a functional fragment” or a “functional variant” in this context includes those fragments and variants that can associate with binding units, e.g., IgM or IgA heavy chain constant regions, to form a pentameric IgM antibody, IgM-like antibody, or IgM-derived binding molecule or a dimeric IgA antibody, IgA-like antibody, or IgA-derived binding molecule, and/or can associate with certain immunoglobulin receptors, e.g., pIgR.

In certain embodiments, the J-chain can be modified, e.g., by introduction of a heterologous moiety, or two or more heterologous moieties, e.g., polypeptides, without interfering with the ability of binding molecule to assemble and bind to its binding target(s). See U.S. Pat. Nos. 9,951,134, 10,400,038, and 10,618,978, and in U.S. Patent Application Publication No. US-2019-0185570, each of which is incorporated herein by reference in its entirety.

Accordingly, a binding molecule provided by this disclosure, including multispecific IgA, IgA-like, IgM, or IgM-like antibodies as described elsewhere herein, can comprise a modified J-chain or functional fragment or variant thereof comprising a heterologous moiety, e.g., a heterologous polypeptide, introduced, e.g., fused or chemically conjugated, into the J-chain or fragment or variant thereof. In certain embodiments, the heterologous polypeptide can be fused to the N-terminus of the J-chain or functional fragment or variant thereof, the C-terminus of the J-chain or functional fragment or variant thereof, or to both the N-terminus and C-terminus of the J-chain or functional fragment or variant thereof. In certain embodiments the heterologous polypeptide can be fused internally within the J-chain or functional fragment or variant thereof. In some embodiments, the heterologous polypeptide can be introduced into the J-chain at or near a glycosylation site. In some embodiments, the heterologous polypeptide can be introduced into the J-chain within about 10 amino acid residues from the C-terminus, or within about 10 amino acids from the N-terminus. In certain embodiments, the heterologous polypeptide can be introduced into the mature human J-chain of SEQ ID NO: 7 between cysteine residues 92 and 101 of SEQ ID NO: 7, or an equivalent location in a J-chain sequence, e.g., a J-chain variant or functional fragment of a J-chain. In a further embodiment, the heterologous polypeptide can be introduced into the mature human J-chain of SEQ ID NO: 7 at or near a glycosylation site. In a further embodiment, the heterologous polypeptide can be introduced into the mature human J-chain of SEQ ID NO: 7 within about 10 amino acid residues from the C-terminus, or within about 10 amino acids from the N-terminus.

In certain embodiments the heterologous moiety can be a peptide or polypeptide sequence fused in frame to the J-chain or chemically conjugated to the J-chain or fragment or variant thereof. In certain embodiments, the heterologous polypeptide is fused to the J-chain or functional fragment thereof via a peptide linker. Any suitable linker can be used, for example the peptide linker can include at least 5 amino acids, at least ten amino acids, and least 20 amino acids, at least 30 amino acids or more, and so on. In certain embodiments, the peptide linker includes least 5 amino acids, but no more than 25 amino acids. In certain embodiments the peptide linker can consist of 5 amino acids, 10 amino acids, 15 amino acids, 20 amino acids, or 25 amino acids. In certain embodiments, the peptide linker consists of GGGGS (SEQ ID NO: 9), GGGGSGGGGS (SEQ ID NO: 10), GGGGSGGGGSGGGGS (SEQ ID NO: 11), GGGGSGGGGSGGGGSGGGGS (SEQ ID NO: 12), or GGGGSGGGGSGGGGSGGGGSGGGGS (SEQ ID NO: 13).

Heterologous moieties to be attached to a J-chain can include, without limitation, a binding moiety, e.g., an antibody or antigen-binding fragment thereof, e.g., a single chain Fv (scFv) molecule, a cytokine, e.g., IL-2 or IL-15 (see, e.g., PCT Application No. PCT US2019/057702, which is incorporated herein by reference in its entirety), a stabilizing peptide that can increase the half-life of the binding molecule, e.g., human serum albumin (HSA) or an HSA binding molecule, or a heterologous chemical moiety such as a polymer.

In some embodiments, a modified J-chain can comprise an antigen-binding domain that can include without limitation a polypeptide capable of specifically binding to a target antigen. In certain embodiments, an antigen-binding domain associated with a modified J-chain can be an antibody or an antigen-binding fragment thereof. In certain embodiments the antigen-binding domain can be a scFv antigen-binding domain or a single-chain antigen-binding domain derived, e.g., from a camelid or condricthoid antibody. In certain embodiments, the target is a target epitope, a target antigen, a target cell, or a target organ.

Variant J-Chains that Confer Increased Serum Half-Life.

In certain embodiments, the J-chain is a functional variant J-chain that includes one or more single amino acid substitutions, deletions, or insertions relative to a reference J-chain identical to the variant J-chain except for the one or more single amino acid substitutions, deletions, or insertions. For example, certain amino acid substitutions, deletions, or insertions can result in the IgM-derived binding molecule exhibiting an increased serum half-life upon administration to a subject animal relative to a reference IgM-derived binding molecule that is identical except for the one or more single amino acid substitutions, deletions, or insertions in the variant J-chain, and is administered using the same method to the same animal species. In certain embodiments the variant J-chain can include one, two, three, or four single amino acid substitutions, deletions, or insertions relative to the reference J-chain. Exemplary J-chains that confer increased serum half-life can be found, e.g., in U.S. Pat. No. 10,899,835, which is incorporated herein by reference in its entirety.

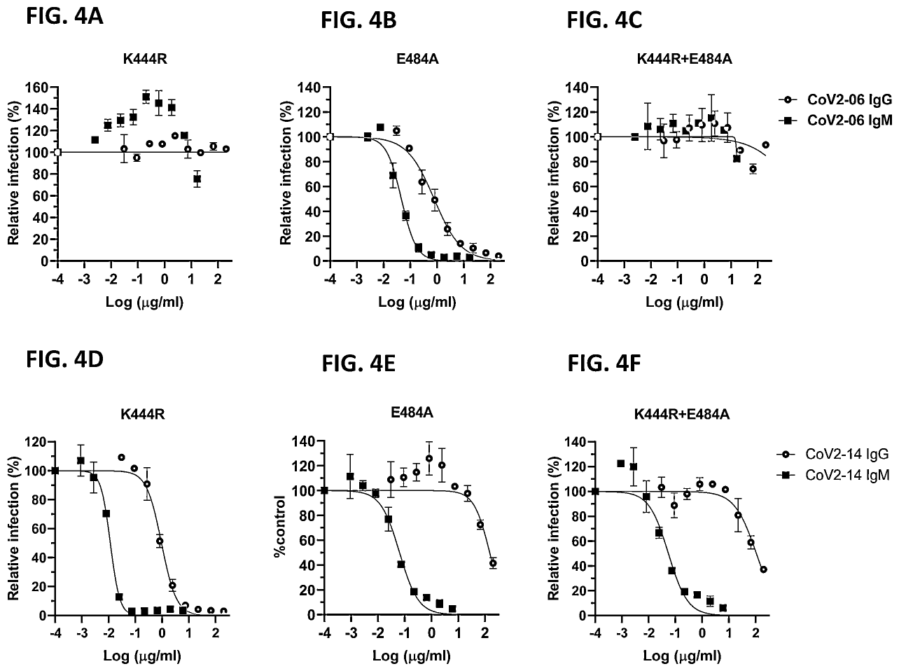

In certain embodiments, the J-chain, such as a modified J-chain, comprises an amino acid substitution at the amino acid position corresponding to amino acid Y102 of the mature wild-type human J-chain (SEQ ID NO: 7). By “an amino acid corresponding to amino acid Y102 of the mature wild-type human J-chain” is meant the amino acid in the sequence of the J-chain, which is homologous to Y102 in the human J-chain. For example, see U.S. Pat. No. 10,899,835, which is incorporated herein by reference in its entirety. The position corresponding to Y102 in SEQ ID NO: 7 is conserved in the J-chain amino acid sequences of at least 43 other species. See FIG. 4 of U.S. Pat. No. 9,951,134, which is incorporated by reference herein. Certain mutations at the position corresponding to Y102 of SEQ ID NO: 7 can inhibit the binding of IgM pentamers comprising the variant J-chain to certain immunoglobulin receptors, e.g., the human or murine Fcαμ receptor, the murine Fcμ receptor, and/or the human or murine polymeric Ig receptor (pIgR).

A multimeric binding molecule comprising a mutation at the amino acid corresponding to Y102 of SEQ ID NO: 7 has an improved serum half-life when administered to an animal than a corresponding multimeric binding molecule that is identical except for the substitution, and which is administered to the same species in the same manner. In certain embodiments, the amino acid corresponding to Y102 of SEQ ID NO: 7 can be substituted with any amino acid. In certain embodiments, the amino acid corresponding to Y102 of SEQ ID NO: 7 can be substituted with alanine (A), serine (S) or arginine (R). In a particular embodiment, the amino acid corresponding to Y102 of SEQ ID NO: 7 can be substituted with alanine. In a particular embodiment the J-chain or functional fragment or variant thereof is a variant human J-chain referred to herein as “J*,” and comprises the amino acid sequence SEQ ID NO: 8.

Wild-type J-chains typically include one N-linked glycosylation site. In certain embodiments, a variant J-chain or functional fragment thereof of a multimeric binding molecule as provided herein includes a mutation within the asparagine(N)-linked glycosylation motif N-X₁-S/T, e.g., starting at the amino acid position corresponding to amino acid 49 (motif N6) of the mature human J-chain (SEQ ID NO: 7) or J* (SEQ ID NO: 8), where N is asparagine, X₁ is any amino acid except proline, and S/T is serine or threonine, and where the mutation prevents glycosylation at that motif. As demonstrated in U.S. Pat. No. 10,899,835, mutations preventing glycosylation at this site can result in the multimeric binding molecule as provided herein, exhibiting an increased serum half-life upon administration to a subject animal relative to a reference multimeric binding molecule that is identical except for the mutation or mutations preventing glycosylation in the variant J-chain, and is administered in the same way to the same animal species.

For example, in certain embodiments the variant J-chain or functional fragment thereof of a pentameric IgM-derived or dimeric IgA-derived binding molecule as provided herein can include an amino acid substitution at the amino acid position corresponding to amino acid N49 or amino acid S51 of SEQ ID NO: 7 or SEQ ID NO: 8, provided that the amino acid corresponding to S51 is not substituted with threonine (T), or where the variant J-chain comprises amino acid substitutions at the amino acid positions corresponding to both amino acids N49 and S51 of SEQ ID NO: 7 or SEQ ID NO: 8. In certain embodiments, the position corresponding to N49 of SEQ ID NO: 7 or SEQ ID NO: 8 is substituted with any amino acid, e.g., alanine (A), glycine (G), threonine (T), serine (S) or aspartic acid (D). In a particular embodiment, the position corresponding to N49 of SEQ ID NO: 7 or SEQ ID NO: 8 can be substituted with alanine (A). In another embodiment, the position corresponding to N49 of SEQ ID NO: 7 or SEQ ID NO: 8 can be substituted with aspartic acid (D). In some embodiments, the position corresponding to S51 of SEQ ID NO: 7 or SEQ ID NO: 8 is substituted with alanine (A) or glycine (G). In some embodiments, the position corresponding to S51 of SEQ ID NO: 7 or SEQ ID NO: 8 is substituted with alanine (A).

### Variant IgM Constant Regions

IgM heavy chain constant regions of a multimeric binding molecule as provided herein can be engineered to confer certain desirable properties to the multimeric binding molecules provided herein. For example, in certain embodiments, IgM heavy chain constant regions can be engineered to confer enhanced serum half-life to multimeric binding molecules as provided herein. Exemplary IgM heavy chain constant region mutations that can enhance serum half-life of an IgM-derived binding molecule are disclosed in U.S. Pat. No. 10,899,835, which is incorporated by reference herein in its entirety. For example, a variant IgM heavy chain constant region of the IgM antibody, IgM-like antibody, or IgM-derived binding molecule as provided herein can include an amino acid substitution at a position corresponding to amino acid S401, E402, E403, R344, and/or E345 of a wild-type human IgM constant region (e.g., SEQ ID NO: 1 or SEQ ID NO: 2). By “an amino acid corresponding to amino acid S401, E402, E403, R344, and/or E345 of a wild-type human IgM constant region” is meant the amino acid in the sequence of the IgM constant region of any species which is homologous to S401, E402, E403, R344, and/or E345 in the human IgM constant region. In certain embodiments, the amino acid corresponding to S401, E402, E403, R344, and/or E345 of SEQ ID NO: 1 or SEQ ID NO: 2 can be substituted with any amino acid, e.g., alanine.

In certain embodiments, an IgM antibody, IgM-like antibody, or other IgM-derived binding molecule as provided herein, can be engineered to exhibit reduced complement-dependent cytotoxicity (CDC) activity to cells in the presence of complement, relative to a reference IgM antibody, IgM-like antibody, or other IgM-derived binding molecule with corresponding reference human IgM constant regions identical, except for the mutations conferring reduced CDC activity. These CDC mutations can be combined with any of the mutations to confer increased serum half-life as provided herein. By “corresponding reference human IgM constant region” is meant a human IgM constant region that is identical to the variant IgM constant region except for the modification or modifications in the constant region affecting CDC activity. In certain embodiments, the variant human IgM constant region includes one or more amino acid substitutions, e.g., in the Cμ3 domain, relative to a wild-type human IgM constant region as described, e.g., in PCT Publication No. WO/2018/187702, which is incorporated herein by reference in its entirety. Assays for measuring CDC are well known to those of ordinary skill in the art, and exemplary assays are described e.g., in PCT Publication No. WO/2018/187702.

In certain embodiments, a variant human IgM constant region conferring reduced CDC activity includes an amino acid substitution corresponding to the wild-type human IgM constant region at position L310, P311, P313, and/or K315 of SEQ ID NO: 1 (human IgM constant region allele IGHM*03) or SEQ ID NO: 2 (human IgM constant region allele IGHM*04). In certain embodiments, a variant human IgM constant region conferring reduced CDC activity includes an amino acid substitution corresponding to the wild-type human IgM constant region at position P311 of SEQ ID NO: 1 or SEQ ID NO: 2. In other embodiments the variant IgM constant region as provided herein contains an amino acid substitution corresponding to the wild-type human IgM constant region at position P313 of SEQ ID NO: 1 or SEQ ID NO: 2. In other embodiments the variant IgM constant region as provided herein contains a combination of substitutions corresponding to the wild-type human IgM constant region at positions P311 of SEQ ID NO: 1 or SEQ ID NO: 2 and P313 of SEQ ID NO: 1 or SEQ ID NO: 2. These proline residues can be independently substituted with any amino acid, e.g., with alanine, serine, or glycine. In certain embodiments, a variant human IgM constant region conferring reduced CDC activity includes an amino acid substitution corresponding to the wild-type human IgM constant region at position K315 of SEQ ID NO: 1 or SEQ ID NO: 2. The lysine residue can be independently substituted with any amino acid, e.g., with alanine, serine, glycine, or aspartic acid. In certain embodiments, a variant human IgM constant region conferring reduced CDC activity includes an amino acid substitution corresponding to the wild-type human IgM constant region at position K315 of SEQ ID NO: 1 or SEQ ID NO: 2 with aspartic acid.

Human and certain non-human primate IgM constant regions typically include five (5) naturally-occurring asparagine (N)-linked glycosylation motifs or sites. As used herein “an N-linked glycosylation motif” comprises or consists of the amino acid sequence N-X₁-S/T, where N is asparagine, X₁ is any amino acid except proline (P), and S/T is serine (S) or threonine (T). The glycan is attached to the nitrogen atom of the asparagine residue. See, e.g., Drickamer K, Taylor M E (2006), Introduction to Glycobiology (2nd ed.). Oxford University Press, USA. N-linked glycosylation motifs occur in the human IgM heavy chain constant regions of SEQ ID NO: 1 or SEQ ID NO: 2 starting at positions 46 (“N1”), 209 (“N2”), 272 (“N3”), 279 (“N4”), and 440 (“N5”). These five motifs are conserved in non-human primate IgM heavy chain constant regions, and four of the five are conserved in the mouse IgM heavy chain constant region. Accordingly, in some embodiments, IgM heavy chain constant regions of a multimeric binding molecule as provided herein comprise 5 N-linked glycosylation motifs: N1, N2, N3, N4, and N5. In some embodiments, at least three of the N-linked glycosylation motifs (e.g., N1, N2, and N3) on each IgM heavy chain constant region are occupied by a complex glycan.

In certain embodiments, at least one, at least two, at least three, or at least four of the N-X₁-S/T motifs can include an amino acid insertion, deletion, or substitution that prevents glycosylation at that motif. In certain embodiments, the IgM-derived multimeric binding molecule can include an amino acid insertion, deletion, or substitution at motif N1, motif N2, motif N3, motif N5, or any combination of two or more, three or more, or all four of motifs N1, N2, N3, or N5, where the amino acid insertion, deletion, or substitution prevents glycosylation at that motif. In some embodiment, the IgM constant region comprises one or more substitutions relative to a wild-type human IgM constant region at positions 46, 209, 272, or 440 of SEQ ID NO: 1 (human IgM constant region allele IGHM*03) or SEQ ID NO: 2 (human IgM constant region allele IGHM*04). See, e.g., U.S. Provisional Application No. 62/891,263, which is incorporated herein by reference in its entirety.

### Polynucleotides and Vectors

In certain embodiments, this disclosure provides a polynucleotide comprising a nucleic acid sequence that encodes a polypeptide subunit of a multimeric binding molecule described herein. In some embodiments, the polynucleotide encodes a polypeptide subunit comprising a heavy chain constant region and at least an antibody VH portion of the SARS-CoV-2-binding domain of a multimeric binding molecule disclosed herein. In some embodiments, the polynucleotide encodes a polypeptide subunit comprising the heavy chain of the multimeric binding molecule. In some embodiments, the polynucleotide encodes a polypeptide subunit comprising a human IgM constant region or fragment thereof fused to the C-terminal end of a VH comprising three immunoglobulin complementarity determining regions HCDR1, HCDR2, and HCDR3, where the HCDR1, HCDR2, and HCDR3 comprise, respectively, the amino acid sequences SEQ ID NO: 15, SEQ ID NO: 16, and SEQ ID NO: 17; SEQ ID NO: 23, SEQ ID NO: 24, and SEQ ID NO: 25; SEQ ID NO: 31, SEQ ID NO: 32, and SEQ ID NO: 33; SEQ ID NO: 39, SEQ ID NO: 40, and SEQ ID NO: 41; SEQ ID NO: 47, SEQ ID NO: 48, and SEQ ID NO: 49; or SEQ ID NO: 55, SEQ ID NO: 56, and SEQ ID NO: 57. In some embodiments, the polynucleotide encodes a polypeptide subunit comprising a human IgM constant region or fragment thereof fused to the C-terminal end of a VH comprising the amino acid sequence SEQ ID NO: 14, SEQ ID NO: 22, SEQ ID NO: 30, SEQ ID NO: 38, SEQ ID NO: 46, or SEQ ID NO: 54, where the CDR regions are defined according to Kabat. In some embodiments, the polynucleotide encodes a polypeptide subunit comprising a human IgM constant region or fragment thereof fused to the C-terminal end of a VH comprising three immunoglobulin complementarity determining regions HCDR1, HCDR2, and HCDR3, where the HCDR1, HCDR2, and HCDR3 comprise, respectively, the amino acid sequences SEQ ID NO: 62, SEQ ID NO: 63, and SEQ ID NO: 64; SEQ ID NO: 68, SEQ ID NO: 69, and SEQ ID NO: 70; SEQ ID NO: 74, SEQ ID NO: 75, and SEQ ID NO: 76; SEQ ID NO: 80, SEQ ID NO: 81, and SEQ ID NO: 82; SEQ ID NO: 86, SEQ ID NO: 87, and SEQ ID NO: 88; or SEQ ID NO: 92, SEQ ID NO: 93, and SEQ ID NO: 94; where the CDR regions are defined according to IMGT. In some embodiments, the polynucleotide encodes a polypeptide subunit comprising a human IgM constant region or fragment thereof fused to the C-terminal end of a VH comprising the amino acid sequence SEQ ID NO: 14, SEQ ID NO: 22, SEQ ID NO: 30, SEQ ID NO: 38, SEQ ID NO: 46, or SEQ ID NO: 54.

In some embodiments, the polynucleotide encodes a polypeptide subunit comprising a light chain constant region and an antibody VL portion of the SARS-CoV-2-binding domain of a multimeric binding molecule disclosed herein. In some embodiments, the polynucleotide encodes a polypeptide subunit comprising the light chain of the multimeric binding molecule. In some embodiments, the polynucleotide encodes a polypeptide subunit comprises a light chain constant region or fragment thereof fused to the C-terminal end of a VL comprising LCDR1, LCDR2, and LCDR3 regions, where the LCDR1, LCDR2, and LCDR3, comprise, respectively, the amino acid sequences SEQ ID NO 19, SEQ ID NO. 20, and SEQ ID NO: 21; SEQ ID NO 27, SEQ ID NO. 28, and SEQ ID NO: 29; SEQ ID NO 35, SEQ ID NO. 36, and SEQ ID NO: 37; SEQ ID NO 43, SEQ ID NO. 44, and SEQ ID NO: 45; SEQ ID NO 51, SEQ ID NO. 52, and SEQ ID NO: 53; or SEQ ID NO 59, SEQ ID NO. 60, and SEQ ID NO: 61; where the CDR regions are defined according to Kabat. In some embodiments, the polynucleotide encodes a polypeptide subunit comprises a light chain constant region or fragment thereof fused to the C-terminal end of a VL comprising LCDR1, LCDR2, and LCDR3 regions, where the LCDR1, LCDR2, and LCDR3, comprise, respectively, the amino acid sequences SEQ ID NO: 65, SEQ ID NO: 66, and SEQ ID NO: 67; SEQ ID NO: 71, SEQ ID NO: 72, and SEQ ID NO: 73; SEQ ID NO: 77, SEQ ID NO: 78, and SEQ ID NO: 79; SEQ ID NO: 83, SEQ ID NO: 84, and SEQ ID NO: 85; EQ ID NO: 89, SEQ ID NO: 90, and SEQ ID NO: 91; or SEQ ID NO: 95, SEQ ID NO: 96, and SEQ ID NO: 97; wherein the CDR regions are defined according to IMGT. In some embodiments, the polynucleotide encodes a polypeptide subunit comprises a light chain constant region or fragment thereof fused to the C-terminal end of a VL comprising the amino acid sequences SEQ ID NO: 18, SEQ ID NO: 26, SEQ ID NO: 34, SEQ ID NO: 42, SEQ ID NO: 50, or SEQ ID NO: 58.

In certain embodiments, this disclosure provides a vector comprising one or more polynucleotides described herein. In some embodiments, the vector further comprises a polynucleotide comprising a nucleic acid sequence that encodes a J-chain or a functional fragment or variant thereof, such as a J-chain, functional fragment or variant thereof described herein.

In certain embodiments, this disclosure provides a composition comprising a first vector and a second vector, where: a) the first vector comprises a polynucleotide comprising a nucleic acid sequence that encodes the heavy chain of the multimeric binding molecule and the second vector comprises a polynucleotide comprising a nucleic acid sequence that encodes the light chain of the multimeric binding molecule, b) the first vector comprises a polynucleotide comprising a nucleic acid sequence that encodes the heavy chain of the multimeric binding molecule and a polynucleotide comprising a nucleic acid sequence that encodes the light chain of the multimeric binding molecule and the second vector comprises a polynucleotide comprising a nucleic acid sequence that encodes a J-chain or a functional fragment or variant thereof, c) the first vector comprises a polynucleotide comprising a nucleic acid sequence that encodes the heavy chain of the multimeric binding molecule and a polynucleotide comprising a nucleic acid sequence that encodes a J-chain or a functional fragment or variant thereof and the second vector comprises a polynucleotide comprising a nucleic acid sequence that encodes the light chain of the multimeric binding molecule, or d) the first vector comprises a polynucleotide comprising a nucleic acid sequence that encodes the light chain of the multimeric binding molecule and a polynucleotide comprising a nucleic acid sequence that encodes a J-chain or a functional fragment or variant thereof and the second vector comprises a polynucleotide comprising a nucleic acid sequence that encodes the heavy chain of the multimeric binding molecule. In certain embodiments, this disclosure provides a composition comprising a first vector, a second vector, and a third vector, where the first vector comprises a polynucleotide comprising a nucleic acid sequence that encodes the heavy chain of the multimeric binding molecule, the second vector comprises a polynucleotide comprising a nucleic acid sequence that encodes the light chain of the multimeric binding molecule, and the third vector comprises a polynucleotide comprising a nucleic acid sequence that encodes a J-chain or a functional fragment or variant thereof.

### Host Cells

In certain embodiments, this disclosure provides a host cell that is capable of producing the multimeric binding molecule as provided herein. In certain embodiments, the host cell comprises one or more vectors, a composition comprising multiple vectors, or polynucleotides disclosed herein. The disclosure also provides a method of producing the multimeric binding molecule as provided herein, where the method comprises culturing the provided host cell, and recovering the multimeric binding molecule.

### Methods of Use

The disclosure further provides a method of treating and/or preventing SARS-CoV-2 infection, e.g., coronavirus disease 2019 (COVID-19) in a subject in need of treatment, where the method includes administering to the subject a therapeutically effective amount of a multimeric binding molecule as provided herein. In certain embodiments the subject is human. By “therapeutically effective dose or amount” or “effective amount” is intended an amount of a multimeric binding molecule that when administered brings about a positive therapeutic response with respect to treatment of subject. Examples of positive therapeutic responses include, without limitation, prevention of respiratory tract colonization or infection by SARS-CoV-2, prevention of SARS-CoV-2 attachment, penetration, and/or replication upon exposure to the virus, prevention of SARS-CoV-2 symptoms, alleviation of SARS-CoV-2 symptoms, reduction of the number of SARS-CoV-2 symptoms, or reduction in the severity of SARS-CoV-2 symptoms. “Symptoms” include, without limitation, one or more of fever, chills, muscle or body aches, fatigue, headache, sore throat, coughing, shortness of breath, difficulty breathing, loss of taste and/or the ability to smell, pneumonia, congestion, nausea, or diarrhea.

Effective doses of the provided multimeric binding molecule depend upon many different factors, including means of administration, target site, physiological state of the subject, whether the subject is human or an animal, other medications administered, and whether treatment is prophylactic or therapeutic. Usually, the subject is a human, but non-human mammals including transgenic mammals can also be treated. Treatment dosages can be titrated using routine methods known to those of skill in the art to optimize safety and efficacy.

In other embodiments, the disclosure provides a method for treating SARS-CoV-2 infection, e.g., Corona Virus Disease 2019 (COVID-19), in a subject in need thereof, where the method includes administering to the subject an effective amount of a multimeric binding molecule as provided herein. In certain embodiments, administration of a multimeric binding molecule as provided herein to a subject results in greater antiviral potency, e.g., greater efficacy at an equivalent dose or the ability to administer a lower dose and achieve equivalent efficacy, than administration of an equivalent amount of a monomeric binding molecule, such as an IgG, binding to the same binding partner. Measurements and indicators of “antiviral potency” are provided elsewhere herein. By “efficacy” is meant the ability of the treatment to, for example, reduce symptoms in an infected subject, reduce the severity of symptoms in an infected subject, prevent symptoms in an infected but asymptomatic subject, reduce the need for hospitalization of an infected subject, reduce the need for auxiliary oxygen in an infected subject or reduce time on a ventilator, reduce the need or the dosage of concomitant medications, reduce the time in intensive care, spare hospital resources, or prevent or reduce transmission from an infected subject to non-infected persons. In certain embodiments the multimeric binding molecule as provided herein can also treat the subject more safely, e.g., by effectively neutralizing naturally-occurring variants with enhanced transmissibility or “escape mutant” viruses. In certain embodiments the monomeric binding molecule includes identical antigen binding domains to the multimeric binding molecule as provided herein. By “an equivalent amount” is meant, e.g., an amount measured by molecular weight, e.g., in total milligrams, or alternatively, a molar equivalent, e.g., where equivalent numbers of molecules are administered.

In other embodiments, the disclosure provides a method for preventing SARS-CoV-2 infection, e.g., Corona Virus Disease 2019 (COVID-19) in a subject in need thereof, e.g., a subject susceptible to SARS-CoV-2 infection or a subject susceptible to more severe COVID-19 symptoms due to proximity to COVID-19 patients, e.g., healthcare providers and/or family members, or due to secondary conditions such as advanced age, diabetes, heart disease, or obesity, where the method includes administering to the subject an effective amount of a multimeric binding molecule as provided herein. In certain embodiments, administration of a multimeric binding molecule as provided herein to a subject results in greater antiviral potency, e.g., as noted above, than administration of an equivalent amount of a monomeric binding polypeptide, such as an IgG, binding to the same binding partner. In certain embodiments the monomeric binding molecule includes identical antigen binding domains to the multimeric binding molecule as provided herein. By “an equivalent amount” is meant, e.g., an amount measured by molecular weight, e.g., in total milligrams, or alternative, a molar equivalent, e.g., where equivalent numbers of molecules are administered.

The subject can be any animal, e.g., a mammal, in need of treatment or prevention, in certain embodiments, the subject is a human subject.

In its simplest form, a preparation to be administered to a subject is multimeric binding molecule as provided herein administered in a conventional dosage form, which can be combined with a pharmaceutical excipient, carrier or diluent as described elsewhere herein.

A multimeric binding molecule of the disclosure can be administered by any suitable method, e.g., parenterally, intraventricularly, orally, by inhalation spray, intranasally, topically, rectally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques.

In certain embodiments the multimeric binding molecule is delivered intranasally, e.g., in an atomized form produced by a suitable spray delivery device, e.g., a MAD NASAL™ Intranasal Mucosal Atomization Device, produced by Teleflex. In certain embodiments, the multimeric binding molecule is delivered orally, e.g., in an atomized form produced by a suitable spray delivery device, e.g., a MAD NASAL™ Intranasal Mucosal Atomization Device, produced by Teleflex. In certain embodiments, the multimeric binding molecule is delivered intranasally and orally, e.g., in an atomized form produced by a suitable spray delivery device, e.g., a MAD NASAL™ Intranasal Mucosal Atomization Device, produced by Teleflex.

In certain embodiments the multimeric binding molecule is delivered via inhalation, e.g., in a nebulized form.

In certain embodiments the multimeric binding molecule is delivered intravenously.

Pharmaceutical Compositions and Administration Methods

The disclosure further provides a composition, e.g., a pharmaceutical composition, comprising a multimeric binding molecule, or two or more multimeric binding molecules, as provided herein. In certain embodiments the composition includes a cocktail of two or more different multimeric binding molecules as described here, that bind to different epitopes on the SARS-CoV-2 RBD. A composition as provided herein can further include a pharmaceutically acceptable carrier and/or excipient and can be formulated so as to be suitable for a desired mode of administration.

Methods of preparing and administering a multimeric binding molecule as provided herein to a subject in need thereof can be determined by a skilled person in view of this disclosure. The route of administration of can be, for example, oral, parenteral, intranasally, by inhalation, by aerosol, or topical. The term parenteral as used herein includes, e.g., intravenous, intraarterial, intraperitoneal, intramuscular, subcutaneous, rectal, or vaginal administration. While these forms of administration are contemplated as suitable forms, another example of a form for administration would be a solution for injection, in particular for intravenous, or intraarterial injection or drip. A suitable pharmaceutical composition can include a buffer (e.g., acetate, phosphate, or citrate buffer), a surfactant (e.g., polysorbate), optionally a stabilizer agent (e.g., human albumin), etc.

A multimeric binding molecule as provided herein can be administered in a pharmaceutically effective amount for the treatment of a subject in need thereof. In this regard, it will be appreciated that the disclosed multimeric binding molecule can be formulated so as to facilitate administration and promote stability of the active agent. Pharmaceutical compositions accordingly can include a pharmaceutically acceptable, non-toxic, sterile carrier such as physiological saline, non-toxic buffers, preservatives, and the like. A pharmaceutically effective amount of a multimeric binding molecule as provided herein means an amount sufficient to achieve effective binding to SARS-CoV-2 and to achieve a therapeutic benefit. Suitable formulations are described in Remington's Pharmaceutical Sciences (Mack Publishing Co.) 16th ed. (1980).

Certain pharmaceutical compositions provided herein can be orally administered in an acceptable dosage form including, e.g., capsules, tablets, aqueous suspensions, or solutions.

Certain pharmaceutical compositions also can be administered by nasal aerosol or inhalation. Such compositions can be prepared as solutions in saline, employing benzyl alcohol or other suitable preservatives, absorption promoters to enhance bioavailability, and/or other conventional solubilizing or dispersing agents. In some embodiments, the pharmaceutical composition is administered by nasal aerosol. In some embodiments, the pharmaceutical composition is for administration by nasal aerosol. In some embodiments, the pharmaceutical composition, such as a pharmaceutical composition for administration by nasal aerosol, comprises a pH adjuster, such as HCl; a buffer; an emulsifier, such as polysorbate or carbomer; sugar or mono- or polyol, such as a monosaccharide (e.g., glucose, dextrose, or fructose), disaccharide (e.g., sucrose, lactose, or maltose), ribose, glycerine, sorbitol, xylitol, inositol, propylene glycol, galactose, mannose, xylose, rhamnose, glutaraldehyde, ethanol, mannitol, polyethylene glycol, glycerol, chitosal, phenylethyl alcohol; a preservative; cellulose, such as microcrystalline cellulose or carboxymethylcellulose; or mixtures thereof.

In some embodiments, the pharmaceutical composition is administered by inhalation. In some embodiments, the pharmaceutical composition is for administration by inhalation. In some embodiments, the pharmaceutical composition, such as a pharmaceutical composition for administration by inhalation, is a dry powder, such as for a dry powder inhaler, or a liquid, such as for a nebulizer, such as an airjet-compressor nebulizer or a mesh-based nebulizer. In some embodiments, the pharmaceutical composition, such as a pharmaceutical composition for administration by inhalation, comprises sugar or mono- or polyol, such as lactose, trelose, mannitol, sorbitol; buffer, such as histidine, proline, or arginine buffer; saline; polysorbate; or mixtures thereof.

The amount of a multimeric binding molecule that can be combined with carrier materials to produce a single dosage form will vary depending, e.g., upon the subject treated and the particular mode of administration. The composition can be administered as a single dose, multiple doses or over an established period of time in an infusion. Dosage regimens also can be adjusted to provide the optimum desired response (e.g., a therapeutic or prophylactic response).

In keeping with the scope of the present disclosure, a multimeric binding molecule as provided herein can be administered to a subject in need of therapy in an amount sufficient to produce a therapeutic effect or a prophylactic effect. A multimeric binding molecule as provided herein can be administered to the subject in a conventional dosage form prepared by combining the multimeric binding molecule of the disclosure with a conventional pharmaceutically acceptable carrier or diluent according to known techniques. The form and character of the pharmaceutically acceptable carrier or diluent can be dictated by the amount of active ingredient with which it is to be combined, the route of administration and other well-known variables.

This disclosure also provides for the use of a multimeric binding molecule as provided herein in the manufacture of a medicament for treating, preventing, or managing COVID-19.

This disclosure employs, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Green and Sambrook, ed. (2012) Molecular Cloning A Laboratory Manual (4th ed.; Cold Spring Harbor Laboratory Press); Sambrook et al., ed. (1992) Molecular Cloning: A Laboratory Manual, (Cold Springs Harbor Laboratory, NY); D. N. Glover and B. D. Hames, eds., (1995) DNA Cloning 2d Edition (IRL Press), Volumes 1-4; Gait, ed. (1990) Oligonucleotide Synthesis (IRL Press); Mullis et al. U.S. Pat. No. 4,683,195; Hames and Higgins, eds. (1985) Nucleic Acid Hybridization (IRL Press); Hames and Higgins, eds. (1984) Transcription And Translation (IRL Press); Freshney (2016) Culture Of Animal Cells, 7th Edition (Wiley-Blackwell); Woodward, J., Immobilized Cells And Enzymes (IRL Press) (1985); Perbal (1988) A Practical Guide To Molecular Cloning; 2d Edition (Wiley-Interscience); Miller and Calos eds. (1987) Gene Transfer Vectors For Mammalian Cells, (Cold Spring Harbor Laboratory); S. C. Makrides (2003) Gene Transfer and Expression in Mammalian Cells (Elsevier Science); Methods in Enzymology, Vols. 151-155 (Academic Press, Inc., N.Y.); Mayer and Walker, eds. (1987) Immunochemical Methods in Cell and Molecular Biology (Academic Press, London); Weir and Blackwell, eds.; and in Ausubel et al. (1995) Current Protocols in Molecular Biology (John Wiley and Sons).

General principles of antibody engineering are set forth, e.g., in Strohl, W. R., and L. M. Strohl (2012), Therapeutic Antibody Engineering (Woodhead Publishing). General principles of protein engineering are set forth, e.g., in Park and Cochran, eds. (2009), Protein Engineering and Design (CDC Press). General principles of immunology are set forth, e.g., in: Abbas and Lichtman (2017) Cellular and Molecular Immunology 9th Edition (Elsevier). Additionally, standard methods in immunology known in the art can be followed, e.g., in Current Protocols in Immunology (Wiley Online Library); Wild, D. (2013), The Immunoassay Handbook 4th Edition (Elsevier Science); Greenfield, ed. (2013), Antibodies, a Laboratory Manual, 2d Edition (Cold Spring Harbor Press); and Ossipow and Fischer, eds., (2014), Monoclonal Antibodies: Methods and Protocols (Humana Press).

All of the references cited above, as well as all references cited herein, are incorporated herein by reference in their entireties.

### ADDITIONAL EMBODIMENTS

Embodiment 1. A multimeric binding molecule comprising two to six bivalent binding units or variants or fragments thereof, wherein each binding unit comprises two IgM or IgA heavy chain constant regions or multimerizing fragments or variants thereof, each associated with a binding domain, wherein three to twelve of the binding domains are identical immunoglobulin antigen binding domains that specifically bind to the SARS-CoV-2 spike (S) protein receptor binding domain (RBD); wherein each identical immunoglobulin antigen binding domain comprises a heavy chain variable region (VH) and a light chain variable region (VL) comprising six immunoglobulin complementarity determining regions HCDR1, HCDR2, HCDR3, LCDR1, LCDR2, and LCDR3, wherein the HCDR1, HCDR2, HCDR3, LCDR1, LCDR2, and LCDR3, comprise, respectively, the amino acid sequences SEQ ID NO: 15, SEQ ID NO: 16, SEQ ID NO: 17, SEQ ID NO 19, SEQ ID NO. 20, and SEQ ID NO: 21; SEQ ID NO: 23, SEQ ID NO: 24, SEQ ID NO: 25, SEQ ID NO 27, SEQ ID NO. 28, and SEQ ID NO: 29; SEQ ID NO: 31, SEQ ID NO: 32, SEQ ID NO: 33, SEQ ID NO 35, SEQ ID NO. 36, and SEQ ID NO: 37; SEQ ID NO: 39, SEQ ID NO: 40, SEQ ID NO: 41, SEQ ID NO 43, SEQ ID NO. 44, and SEQ ID NO: 45; SEQ ID NO: 47, SEQ ID NO: 48, SEQ ID NO: 49, SEQ ID NO 51, SEQ ID NO. 52, and SEQ ID NO: 53; or SEQ ID NO: 55, SEQ ID NO: 56, SEQ ID NO: 57, SEQ ID NO 59, SEQ ID NO. 60, and SEQ ID NO: 61; wherein the CDR regions are defined according to Kabat; and wherein the multimeric binding molecule has greater antiviral potency against SARS-CoV-2 than a bivalent reference IgG antibody comprising two of the binding domains that specifically bind to the SARS-CoV-2 S protein RBD.

Embodiment 2. A multimeric binding molecule comprising two to six bivalent binding units or variants or fragments thereof, wherein each binding unit comprises two IgM or IgA heavy chain constant regions or multimerizing fragments or variants thereof, each associated with a binding domain, wherein three to twelve of the binding domains are identical immunoglobulin antigen binding domains that specifically bind to the SARS-CoV-2 spike (S) protein receptor binding domain (RBD); wherein each identical immunoglobulin antigen binding domain comprises a heavy chain variable region (VH) and a light chain variable region (VL) comprising six immunoglobulin complementarity determining regions HCDR1, HCDR2, HCDR3, LCDR1, LCDR2, and LCDR3, wherein the HCDR1, HCDR2, HCDR3, LCDR1, LCDR2, and LCDR3, comprise, respectively, the amino acid sequences SEQ ID NO: 62, SEQ ID NO: 63, SEQ ID NO: 64, SEQ ID NO: 65, SEQ ID NO: 66, and SEQ ID NO: 67; SEQ ID NO: 68, SEQ ID NO: 69, SEQ ID NO: 70, SEQ ID NO: 71, SEQ ID NO: 72, and SEQ ID NO: 73; SEQ ID NO: 74, SEQ ID NO: 75, SEQ ID NO: 76, SEQ ID NO: 77, SEQ ID NO: 78, and SEQ ID NO: 79; SEQ ID NO: 80, SEQ ID NO: 81, SEQ ID NO: 82, SEQ ID NO: 83, SEQ ID NO: 84, and SEQ ID NO: 85; SEQ ID NO: 86, SEQ ID NO: 87, SEQ ID NO: 88, SEQ ID NO: 89, SEQ ID NO: 90, and SEQ ID NO: 91; or SEQ ID NO: 92, SEQ ID NO: 93, SEQ ID NO: 94, SEQ ID NO: 95, SEQ ID NO: 96, and SEQ ID NO: 97; wherein the CDR regions are defined according to IMGT; and wherein the multimeric binding molecule has greater antiviral potency against SARS-CoV-2 than a comprising two of the binding domains that specifically bind to the SARS-CoV-2 S protein RBD.

Embodiment 3. The multimeric binding molecule of embodiment 1 or embodiment 2, wherein the bivalent reference IgG antibody comprises two identical antigen binding domains comprising the VH and VL amino acid sequences SEQ ID NO: 14 and SEQ ID NO: 18, SEQ ID NO: 22 and SEQ ID NO: 26, SEQ ID NO: 30 and SEQ ID NO: 34, SEQ ID NO: 38 and SEQ ID NO: 42, SEQ ID NO: 46 and SEQ ID NO: 50, or SEQ ID NO: 54 and SEQ ID NO: 58, respectively.

Embodiment 4. The multimeric binding molecule of any one of embodiments 1 to 3, wherein the VH and VL comprise the amino acid sequences SEQ ID NO: 14 and SEQ ID NO: 18, SEQ ID NO: 22 and SEQ ID NO: 26, SEQ ID NO: 30 and SEQ ID NO: 34, SEQ ID NO: 38 and SEQ ID NO: 42, SEQ ID NO: 46 and SEQ ID NO: 50, or SEQ ID NO: 54 and SEQ ID NO: 58, respectively.

Embodiment 5. The multimeric binding molecule of any one of embodiments 1, 3, or 4, wherein the HCDR1, HCDR2, HCDR3, LCDR1, LCDR2, and LCDR3, comprise, respectively, the amino acid sequences SEQ ID NO: 15, SEQ ID NO: 16, SEQ ID NO: 17, SEQ ID NO 19, SEQ ID NO. 20, and SEQ ID NO: 21, or SEQ ID NO: 39, SEQ ID NO: 40, SEQ ID NO: 41, SEQ ID NO 43, SEQ ID NO. 44, and SEQ ID NO: 45; wherein the CDR regions are defined according to Kabat.

Embodiment 6. The multimeric binding molecule of any one of embodiments 2 to 4, wherein the HCDR1, HCDR2, HCDR3, LCDR1, LCDR2, and LCDR3, comprise, respectively, the amino acid sequences SEQ ID NO: 62, SEQ ID NO: 63, SEQ ID NO: 64, SEQ ID NO: 65, SEQ ID NO: 66, and SEQ ID NO: 67; or SEQ ID NO: 80, SEQ ID NO: 81, SEQ ID NO: 82, SEQ ID NO: 83, SEQ ID NO: 84, and SEQ ID NO: 85; wherein the CDR regions are defined according to IMGT.

Embodiment 7. The multimeric binding molecule of embodiment 5 or embodiment 6, wherein the VH and VL comprise the amino acid sequences SEQ ID NO: 14 and SEQ ID NO: 18 or SEQ ID NO: 38 and SEQ ID NO: 42, respectively.

Embodiment 8. The multimeric binding molecule of embodiment 7, wherein the VH and VL of the multimeric binding molecule comprise the amino acid sequences SEQ ID NO: 14 and SEQ ID NO: 18, respectively, and wherein the VH and VL of the bivalent reference IgG antibody comprise the amino acid sequences SEQ ID NO: 14 and SEQ ID NO: 18, respectively.

Embodiment 9. The multimeric binding molecule of embodiment 7, wherein the VH and VL of the multimeric binding molecule comprise the amino acid sequences SEQ ID NO: 38 and SEQ ID NO: 42, respectively, and wherein the VH and VL of the bivalent reference IgG antibody comprise the amino acid sequences SEQ ID NO: 38 and SEQ ID NO: 42, respectively.

Embodiment 10. The multimeric binding molecule of any one of embodiments 1 to 9, wherein the greater antiviral potency against SARS-CoV-2 comprises a) inhibition of binding of the SARS-CoV-2 spike protein to its receptor angiotensin-converting enzyme 2 (ACE2) at a lower 50% effective concentration (EC50) than the bivalent reference IgG antibody, b) inhibition of binding of the SARS-CoV-2 spike protein to ACE2 under conditions where the bivalent reference IgG antibody cannot inhibit binding, c) neutralization of SARS-CoV-2 infectivity at a lower EC50 than the bivalent reference IgG antibody, d) neutralization of SARS-CoV-2 infectivity under conditions where the bivalent reference IgG antibody cannot neutralize SARS-CoV-2 infectivity, e) protection against SARS-CoV-2 infection in a therapeutic animal model at a lower 50% effective dose (ED50) than the bivalent IgG antibody, f) protection against SARS-CoV-2 infection in the therapeutic animal model under conditions where the bivalent reference IgG antibody cannot protect, g) protection against SARS-CoV-2 infection in a prophylactic animal model at a lower ED50 than the bivalent IgG antibody, h) protection against SARS-CoV-2 infection in the prophylactic animal model under conditions where the bivalent reference IgG antibody cannot protect, or i) any combination thereof.

Embodiment 11. The multimeric binding molecule of embodiment 10, wherein the binding molecule can neutralize infectivity SARS-CoV-2 at a lower EC50 than the bivalent reference IgG antibody or can neutralize infectivity of SARS-CoV-2 under conditions where the bivalent reference IgG antibody cannot neutralize.

Embodiment 12. The multimeric binding molecule of embodiment 11, wherein the EC50 is at least two-fold, at least five-fold, at least ten-fold, at least fifty-fold, at least 100-fold, at least 500-fold, or at least 1000-fold lower than the EC50 of the bivalent IgG antibody.

Embodiment 13. The multimeric binding molecule of embodiment 11, wherein the conditions where the bivalent reference IgG antibody cannot neutralize comprises neutralization of an antibody-resistant variant of SARS-CoV-2.

Embodiment 14. The multimeric binding molecule of embodiment 13, wherein the antibody resistant variant of SARS-CoV-2 comprises an “escape mutant” of a SARS-CoV-2 virus that arose following contact with the bivalent reference IgG antibody.

Embodiment 15. The multimeric binding molecule of any one of embodiments 10 to 14, wherein the binding molecule can confer protection against SARS-CoV-2 infection in a therapeutic or prophylactic animal model at a lower 50% effective dose (ED50) than the bivalent reference IgG antibody, or wherein the binding molecule can confer protection against SARS-CoV-2 infection in a therapeutic or prophylactic animal model under conditions where the bivalent reference IgG antibody cannot protect.

Embodiment 16. The multimeric binding molecule of embodiment 15, wherein the conditions where the bivalent reference IgG antibody cannot protect against SARS-CoV-2 infection comprises a virus challenge with an antibody-resistant variant of SARS-CoV-2.

Embodiment 17. The multimeric binding molecule of embodiment 16, wherein the antibody resistant variant of SARS-CoV-2 comprises an “escape mutant” of a SARS-CoV-2 virus that arose following contact with the bivalent reference IgG antibody.

Embodiment 18. The multimeric binding molecule of any one of embodiments 10 to 17, wherein the multimeric binding molecule reduces, inhibits, or blocks the SARS-CoV-2 S protein from binding to ACE2 at a lower EC50 than the bivalent reference IgG antibody or reduces, inhibits, or blocks the SARS-CoV-2 S protein from binding to ACE2 under conditions where the bivalent reference IgG antibody cannot reduce, inhibit, or block the SARS-CoV-2 S protein from binding to ACE2.

Embodiment 19. The multimeric binding molecule of any one of embodiments 1 to 18, wherein the immunoglobulin antigen-binding domains are human immunoglobulin antigen-binding domains.

Embodiment 20. The multimeric binding molecule of embodiment any one of embodiments 1 to 19, wherein each binding unit comprises two heavy chains comprising the VH and two light chains comprising the VL.

Embodiment 21. The multimeric binding molecule of any one of embodiments 1 to 20, comprising two or four bivalent IgA or IgA-like binding units and a J chain or functional fragment or variant thereof, wherein each binding unit comprises two IgA heavy chain constant regions or multimerizing fragments or variants thereof, each comprising an IgA Cα3 domain and an IgA tailpiece domain.

Embodiment 22. The multimeric binding molecule of embodiment 21, which is a dimeric binding molecule comprising two bivalent IgA or IgA-like binding units.

Embodiment 23. The multimeric binding molecule of embodiment 21 or embodiment 22, wherein each IgA heavy chain constant region or multimerizing fragment or variant thereof further comprises a Cα1 domain, a Cα2 domain, an IgA hinge region, or any combination thereof.

Embodiment 24. The multimeric binding molecule of any one of embodiments 21 to 23, wherein the IgA heavy chain constant regions or multimerizing fragments or variants thereof are human IgA constant regions.

Embodiment 25. The multimeric binding molecule of any one of embodiments 21 to 24, wherein the human IgA constant regions comprise the human IgA1 constant region amino acid sequence of SEQ ID NO: 3, the human IgA2 constant region amino acid sequence of SEQ ID NO: 4, or a multimerizing fragment or variant of SEQ ID NO: 3 or SEQ ID NO: 4.

Embodiment 26. The multimeric binding molecule of any one of embodiments 21 to 24, wherein each binding unit comprises two IgA heavy chains each comprising a VH situated amino terminal to the IgA constant region or multimerizing fragment thereof, and two immunoglobulin light chains each comprising a VL situated amino terminal to an immunoglobulin light chain constant region.

Embodiment 27. The multimeric binding molecule of any one of embodiments 1 to 20, comprising five or six bivalent IgM or IgM-like binding units, wherein each binding unit comprises two IgM heavy chain constant regions or multimerizing fragments or variants thereof, each comprising an IgM Cμ4 and IgM tailpiece domain.

Embodiment 28. The multimeric binding molecule of embodiment 27, wherein each IgM heavy chain constant region or multimerizing fragment or variant thereof further comprises a Cμ1 domain, a Cμ2 domain, a Cμ3 domain, or any combination thereof.

Embodiment 29. The multimeric binding molecule of embodiment 27 or embodiment 28, wherein the IgM heavy chain constant regions or multimerizing fragments or variants thereof are human IgM constant regions.

Embodiment 30. The multimeric binding molecule of embodiment 29, wherein the IgM heavy chain constant regions each comprise the amino acid sequence SEQ ID NO: 1, SEQ ID NO: 2, or a multimerizing fragment or variant thereof.

Embodiment 31. The multimeric binding molecule of any one of embodiments 27 to 30, wherein each binding unit comprises two IgM heavy chains each comprising a VH situated amino terminal to the IgM constant region or multimerizing fragment or variant thereof, and two immunoglobulin light chains each comprising a VL situated amino terminal to an immunoglobulin light chain constant region.

Embodiment 32. The multimeric binding molecule of any one of embodiments 27 to 31, wherein the IgM constant regions each comprise one or more amino acid substitutions at positions corresponding to amino acids 310, 311, 313, and/or 315 of SEQ ID NO: 1 or SEQ ID NO: 2, and wherein the multimeric binding molecule exhibits reduced complement-dependent cytotoxicity (CDC) activity to cells in the presence of complement, relative to a reference binding molecule that is identical except for the one or more amino acid substitutions.

Embodiment 33. The multimeric binding molecule of any one of embodiments 27 to 32, wherein the IgM constant regions each comprise one or more substitutions at positions corresponding to N46, N209, N272, or N440 of SEQ ID NO: 1 or SEQ ID NO: 2, wherein the one or more amino acid substitutions prevent asparagine (N)-linked glycosylation.

Embodiment 34. The multimeric binding molecule of any one of embodiments 27 to 33 which is pentameric, and further comprises a J-chain or functional fragment or variant thereof.

Embodiment 35. The multimeric binding molecule of any one of embodiments 21 to 26 or 34, which can transport across vascular endothelial cells via J-chain binding to the polymeric Ig receptor (PIgR).

Embodiment 36. The multimeric binding molecule of any one of embodiments 21 to 26 or 34 or 35, further comprising a secretory component, or fragment or variant thereof.

Embodiment 37. The multimeric binding molecule of any one of embodiments 21 to 26 or 34 to 36, wherein the J-chain or functional fragment or variant thereof further comprises a heterologous polypeptide, wherein the heterologous polypeptide is directly or indirectly fused to the J-chain or functional fragment or variant thereof.

Embodiment 38. The multimeric binding molecule of embodiment 37, wherein the heterologous polypeptide is fused to the J-chain or fragment thereof via a peptide linker.

Embodiment 39. The multimeric binding molecule of embodiment 37 or embodiment 38, wherein a heterologous polypeptide is fused to the N-terminus of the J-chain or fragment or variant thereof, the C-terminus of the J-chain or fragment or variant thereof, or to both the N-terminus and C-terminus of the J-chain or fragment or variant thereof, wherein the heterologous polypeptides fused to both the N-terminus and C-terminus can be the same or different.

Embodiment 40. The multimeric binding molecule of any one of embodiments 37 to 39, wherein the heterologous polypeptide can influence the absorption, distribution, metabolism and/or excretion (ADME) of the multimeric binding molecule.

Embodiment 41. The multimeric binding molecule of embodiment 40, wherein the heterologous polypeptide comprises an albumin or an albumin binding domain.

Embodiment 42. The multimeric binding molecule of embodiment 41, wherein the heterologous polypeptide comprises human serum albumin.

Embodiment 43. A composition comprising the multimeric binding molecule of any one of embodiments 1 to 42.

Embodiment 44. A composition comprising two or more nonidentical multimeric binding molecules according to any one of embodiments 1 to 42, wherein the two or more multimeric binding molecules bind to different epitopes of the SARS-CoV-2 spike (S) protein receptor binding domain (RBD).

Embodiment 45. A polynucleotide comprising a nucleic acid sequence that encodes a polypeptide subunit of the binding molecule of any one of embodiments 1 to 42.

Embodiment 46. A vector comprising the polynucleotide of embodiment 45.

Embodiment 47. A host cell comprising the polynucleotide of embodiment 45, or the vector of embodiment 46, wherein the host cell can express the multimeric binding molecule of any one of embodiments 1 to 42, or a subunit thereof.

Embodiment 48. A method of producing the multimeric binding molecule of any one of embodiments 1 to 42, comprising culturing the host cell of embodiment 47, and recovering the multimeric binding molecule.

Embodiment 49. The method of embodiment 48, further comprising contacting the multimeric binding molecule with a secretory component, or fragment or variant thereof.

Embodiment 50. A method for treating SARS-CoV-2 infection in a subject comprising administering to a subject in need of treatment an effective amount of the multimeric binding molecule of any one of embodiments 1 to 42, wherein the multimeric binding molecule has greater antiviral potency against SARS-CoV-2 than a bivalent reference IgG antibody comprising two of the binding domains that specifically bind to the SARS-CoV-2 S protein RBD.

Embodiment 51. A method for preventing SARS-CoV-2 infection in a subject, comprising administering to a subject susceptible to SARS-CoV-2 infection an effective amount of the multimeric binding molecule of any one of embodiments 1 to 42, wherein the multimeric binding molecule has greater antiviral potency against SARS-CoV-2 than a bivalent reference IgG antibody comprising two of the binding domains that specifically bind to the SARS-CoV-2 S protein RBD.

Embodiment 52. The method of embodiment 50 or embodiment 51, wherein the SARS-CoV-2 infection is coronavirus disease 2019 (COVID-19).

Embodiment 53. The method of any one of embodiments 50 to 52, wherein the subject is human.

Embodiment 54. The method of any one of embodiments 50 to 53, wherein the administering comprises intravenous, subcutaneous, intramuscular, intranasal, and/or inhalation administration.

Embodiment 55. The method of embodiment 54, wherein the administering comprises intranasal administration.

Embodiment 56. The method of embodiment 54 or embodiment 55, wherein the administering comprises inhalation administration.

Embodiment 57. The method of any one of embodiments 54 to 56, wherein the administering comprises intravenous infusion.

The following examples are offered by way of illustration and not by way of limitation.

### EXAMPLES

### Example 1: Multimeric Anti-SARS-CoV-2 Antibody Generation

The VH and VL regions of six human anti-SARS-CoV-2 antibodies, anti-SARS-CoV2-06 (“CoV2-06,” SEQ ID NO: 14 and SEQ ID NO: 18, respectively), anti-SARS-CoV2-09 (“CoV2-09,” SEQ ID NO: 22 and SEQ ID NO: 26, respectively), anti-SARS-CoV2-12 (“CoV2-12,” SEQ ID NO: 30 and SEQ ID NO: 34, respectively), anti-SARS-CoV2-14 (“CoV2-14,” SEQ ID NO: 38 and SEQ ID NO: 42, respectively), anti-SARS-CoV2-16 (“CoV2-16,” SEQ ID NO: 46 and SEQ ID NO: 50, respectively), and anti-SARS-CoV2-26 (“CoV2-26,” SEQ ID NO: 54 and SEQ ID NO: 58, respectively) (U.S. application 63/015,257), were incorporated into IgM, IgA1, and IgA2m2 formats (each with an exemplary human J-chain, SEQ ID NO: 7) and IgG format according to standard cloning protocols. Control antibody binding domains from anti SARS-CoV antibodies CR3022 (VH: SEQ ID NO: 98, VL (kappa): SEQ ID NO: 99) and CR3014 (VH: SEQ ID NO: 100, VL: SEQ ID NO: 101), were likewise incorporated into IgM, IgA1, and IgA2m2 formats (each with an exemplary human J-chain, SEQ ID NO: 7) and IgG format according to standard cloning protocols. CR3022 IgG binds to SARS-CoV-2 while CR3014 IgG does not (Tian et al. Emerging Microbes & Infections, 2020, doi: 10.1080/22221751.2020.1729069).

The IgM, IgA, and IgG antibody constructs were expressed in in Expi293 or CHO cells. The IgM antibodies were purified according to methods described in Keyt, B., et al. Antibodies: 9:53, doi: 10.3390/antib9040053 (2020). The IgA and IgG antibodies were purified by affinity chromatography. The IgM antibodies assembled as pentamers with a J-chain, except for Anti-SARS-CoV2-26, and the IgA antibodies assembled as dimers with a J-chain (data not shown). The light chains of the original isolated CoV2-06, CoV2-09, CoV2-14, CoV2-16, and CoV2-26 IgG antibodies were lambda light chains (Ku, Z, et al., Nature Comm. 12:469 doi:10.1038/s41467-020-20789-7, 2021). For the assays described in Examples 2-4 and Example 6, FIG. 4A-F, the light chains for these antibodies were constructed as lambda-VL-kappa-CL hybrid light chains. The CoV2-06, CoV2-09, CoV2-14, CoV2-16, and CoV2-26 antibodies used in the remaining Examples, however, contained full lambda light chains. The heavy chain of CoV2-14 IgM comprises the amino acid sequence SEQ ID NO: 105, the light chain of CoV2-14 IgM comprises the amino acid sequence SEQ ID NO: 106, and the J-chain of CoV2-14 IgM comprises the amino acid sequence SEQ ID NO: 7.

### Example 2: Anti-SARS-CoV-2 Binding Measured by ELISA

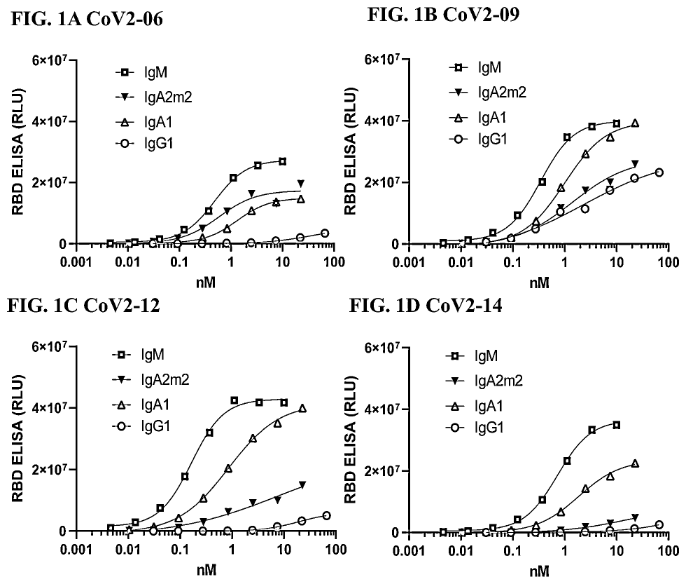

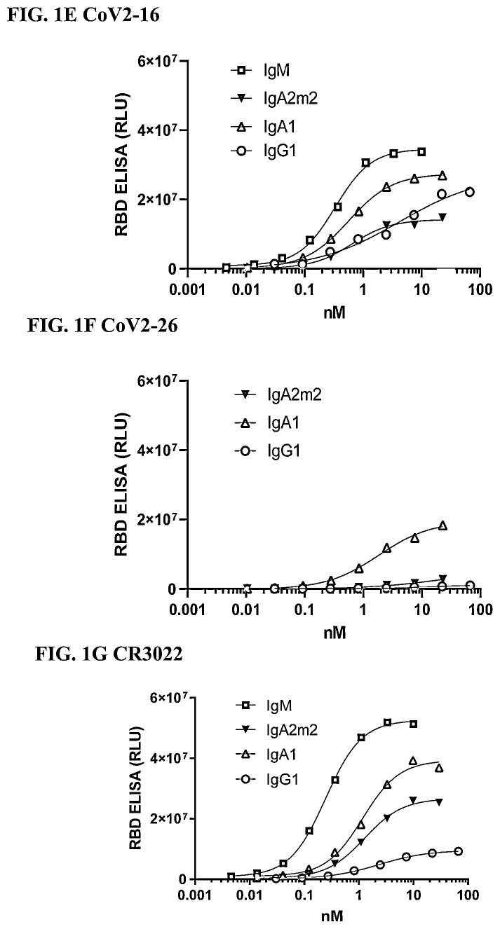

Binding of CoV2-06, CoV2-09, CoV2-12, CoV2-14, CoV2-16, CoV2-26, and CR3022 constructs described in Example 1 to the receptor-binding domain (RBD) of the SARS-CoV-2 spike protein was measured in ELISA assays as follows. Ninety-six-well white polystyrene ELISA plates (Pierce 15042) were coated with 100 μL per well of 0.5 μg/mL recombinant SARS-CoV-2 Receptor Binding Domain (RBD) with a his tag (ATUM, Cat. 65639.1.a) overnight at 4° C. Plates were then washed 5 times with 0.05% PBS-Tween and blocked with 2% BSA-PBS. After blocking, 100 μL of serial dilutions of CoV2-06, CoV2-09, CoV2-12, CoV2-14, CoV2-16, and CR3022 IgM, IgA1, IgA2m2, or IgG and CoV2-26 IgA1, IgA2m2, or IgG; standards; or isotype controls were added to the wells and incubated at room temperature for 2 hours. The plates were then washed 10 times and incubated with HRP conjugated mouse anti-human kappa (Southern Biotech, 9230-05. 1:6000 diluted in 2% BSA-PBS) for 30 min. After 10 final washes using 0.05% PBS-Tween, the signals were generated using SuperSignal chemiluminescent substrate (ThermoFisher, 37070). Luminescent data were collected on an EnVision plate reader (Perkin-Elmer) and analyzed with GraphPad Prism using a 4-parameter logistic model. Binding to SARS-CoV-2 RBD by the CoV2-06, CoV2-09, CoV2-12, CoV2-14, CoV2-16, CoV2-26, and CR3022 antibodies is shown in FIGS. 1A-1G, respectively. The EC50 values (in nM) for binding are provided in Table 2.

TABLE 2
RBD Binding

| | EC₅₀ Values for Binding to the RBD by ELISA (nM) | | | | | | |
|---|---|---|---|---|---|---|---|
| | CoV2-06 | CoV2-09 | CoV2-12 | CoV2-14 | CoV2-26 | CoV2-26 | CR3022 |
| IgG1 | 41.0 | 2.3 | 19.0 | >60 | 4.2 | 13.0 | 2.3 |
| IgA1 | 1.3 | 1.0 | 0.9 | 1.9 | 0.6 | 1.8 | 1.2 |
| IgA2m2 | 0.6 | 1.4 | 8.1 | 14.0 | 0.6 | 38.0 | 1.3 |
| IgM | 0.5 | 0.3 | 0.2 | 0.7 | 0.3 | NA | 0.2 |

### Example 3: SARS-CoV-2 Neutralization

A fluorescent SARS-CoV2 neutralization assay was used to determine how the IgM, IgA1, IgA2m2, and IgG1 formats of CoV2-06, CoV2-09, CoV2-12, CoV2-14, and CoV2-16, and IgA1, IgA2m2, and IgG1 formats of CoV2-26 affect neutralization of SARS-CoV-2.

The neutralization assay was performed using the mNeonGreen SARS-CoV-2 live virus (SARS-CoV-2-mNG) generally as described in Muruato et al. (Nat Commun 11, 4059 doi: 10.1038/s41467-020-17892-0 (2020)).

First, initial screens of all antibodies were carried out on all antibodies at 1 μg/mL or 0.1 μg/mL. Controls included IgM, IgA2, and IgG versions of an anti-CD20 antibody, and IgM, IgA1, IgA2, and IgG versions of anti-SARS-CoV antibodies CR3014 (van den Brink, E. N. et al., J. Virol. 2005 February; 79(3):1635-1644. doi: 10.1128/JVI.79.3.1635-1644.2005) and CR3022 (ter Meulen, J, et al., PLoS Med. 2006 July; 3(7):e237. doi: 10.1371/journal.pmed.0030237). CR3022 IgG was recently demonstrated to also bind SARS-CoV-2, but CR3014 did not bind to SARS-CoV-2 (Tian et al. Emerging Microbes & Infections, 2020, doi: 10.1080/22221751.2020.1729069). For these initial screens, a total of 1.5×10⁴ Vero cells were plated into each well of a black transparent flat-bottom 96-well plate (Greiner Bio-One; Cat #655090). The next day, the antibodies were mixed with an equal volume of SARS-CoV-2-mNeonGreen (mNG) virus (MOI=0.5) to achieve final dilutions of 1 μg/mL or 0.1 μg/mL. After 1 h incubation at 37° C., the antibody-virus complexes were inoculated onto the Vero cells. At 20 h post-infection, nuclei were stained by the addition of Hoechst 33342 (Thermo Fisher Scientific) to a final concentration of 10 nM. Fluorescent images were acquired using a Cytation 7 multimode reader (BioTek). Total cells (in blue) and mNG-positive cells (in green) were counted, and the infection rate was calculated. The relative infection rates were calculated by normalizing the infection rate of each well to that of control wells (no antibody treatment).

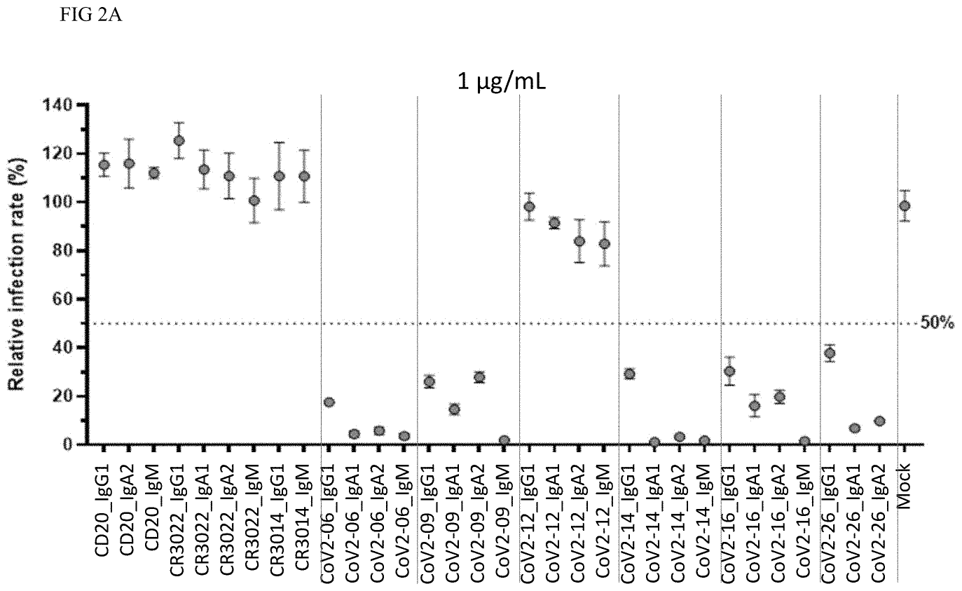

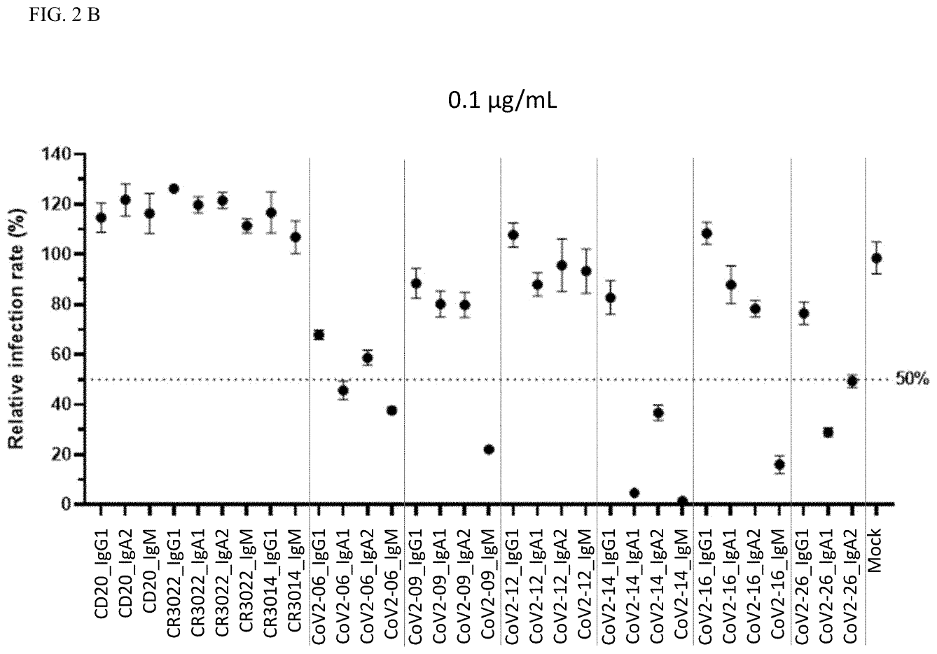

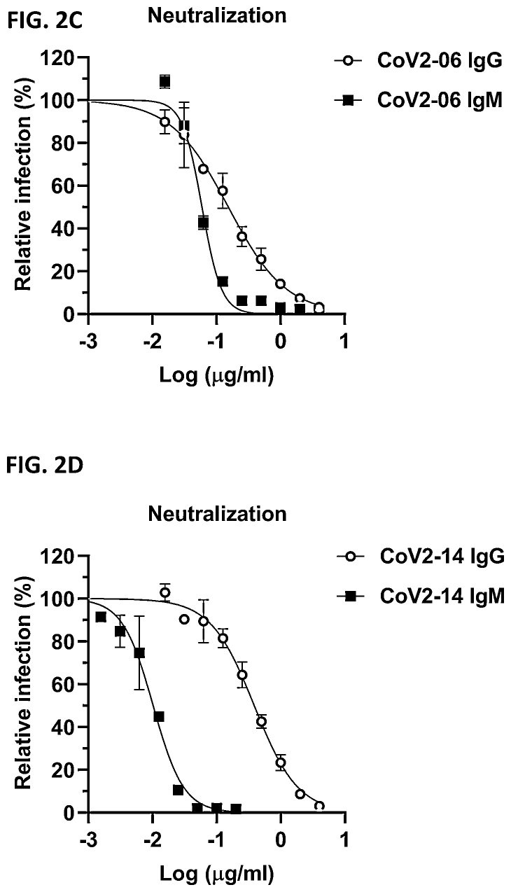

The relative infection rates of SARS-CoV-2-mNG at 1 μg/mL and 0.1 μg/mL are shown in FIGS. 2A and 2B, respectively. All formats of anti-CD20, CR3022, and CR3014 did not neutralize SARS-CoV-2. In the neutralization of live SARS-CoV-2, all IgM and IgA1 mAbs exhibited stronger activities than the parental IgG1 mAbs at 1 μg/ml and 0.1 μg/ml.

In a second assay, neutralization titrations of IgM and IgG versions of CoV2-06 and CoV2-14 were performed. A total of 1.5×10⁴ Vero cells were plated into each well of a black transparent flat-bottom 96-well plate (Greiner Bio-One; Cat #655090). On the next day, antibodies (serial dilutions) were mixed with an equal volume of SARS-CoV-2-mNG virus (MOI=0.5). After 1 h incubation at 37° C., the antibody-virus complexes were inoculated into Vero cells. At 20 h post-infection, nuclei were stained by the addition of Hoechst 33342 (Thermo Fisher Scientific) to a final concentration of 10 nM. Fluorescent images were acquired using a Cytation 7 multimode reader (BioTek). Total cells (in blue) and mNG-positive cells (in green) were counted, and the infection rate was calculated. The relative infection rates were calculated by normalizing the infection rate of each well to that of control wells (no antibody treatment).

The relative infection rates of the IgG and IgM antibodies are shown in FIG. 2C (CoV2-06) and FIG. 2D (CoV2-14). The EC₅₀ values for the CoV2-06 IgM and IgG antibodies and CoV2-14 IgM and IgG antibodies are shown in Table 3 and Table 4, respectively. The CoV2-06 IgM and CoV2-14 IgM antibodies showed stronger neutralization activity against live SARS-CoV-2 than the corresponding IgG antibodies, respectively.

### Example 4: Further Binding Comparisons of CoV2-06 and CoV2-14 IgM Antibodies

The IgM and IgG versions of CoV2-06 and CoV2-14 were further tested in an ELISA binding assay and an antibody avidity assay, as follows. For the ELISA assay, high binding ELISA plates were coated with recombinant Spike protein (1 μg/ml, Sino Biologicals Cat #40589-V08B1) at 4° C. overnight and blocked with 5% skim milk at 37° C. for 2 hours. Antibodies were serially diluted in 1% skim milk and added at a volume of 100 μl per well for incubation at 37° C. for 2h. The anti-human IgG Fab2 HRP-conjugated F(ab′)₂ fragment Goat Anti-Human IgA+IgG+IgM (H+L) antibody (Jackson ImmunoResearch, Cat #109-036-064) was diluted 1:5000 and added at a volume of 100 μl per well for incubation at 37° C. for 1 h. The plates were washed 3-5 times with PBST (0.05% Tween-20) between incubation steps. TMB substrate was added 100 μl per well for color development. The reaction was stopped by adding 50 μl per well 2M H₂SO₄. The OD450 nm was read by a SpectraMax microplate reader. The data points were plotted by GraphPad Prism8, and the EC₅₀ values were calculated using a three-parameter nonlinear model.

The avidity (apparent affinity) of the antibodies to the whole spike (S) protein was performed on the Pall ForteBio Octet RED96 system. The S protein (His-tagged, 15m/m1) was captured on the Ni+ NTA biosensor. Following 10 s of baseline in kinetics buffer, the sensors were dipped in three-fold serially diluted antibodies (0.12˜90 nM) for 200s to record association kinetics. Then, the sensors were dipped into kinetics buffer for 400s to record dissociation kinetics. ForteBio's data analysis software was used to fit the KD data using the global fitting method.

The ELISA and avidity results for the CoV2-06 IgM and IgG antibodies and CoV2-14 IgM and IgG antibodies are shown in Table 3 and Table 4, respectively. For the CoV2-06 IgM antibody, the change of KD was over 750-fold (<0.001 nM vs. 0.75 nM) over the CoV2-06 IgG antibody, and the change of EC₅₀ was 17.6-fold (0.10 nM vs. 1.76 nM). For the CoV2-14 IgM antibody, the change of KD was 13.6-fold (0.102 nM vs. 1.39 nM), and the change of EC₅₀ was 135-fold (0.046 nM vs. 6.22 nM). These results indicate that engineered IgMs have improved binding and neutralization compared to IgGs.

TABLE 3
Binding and Neutralization Characteristics of CoV2-06 Antibodies

| | | Avidity to spike | | | | |
|---|---|---|---|---|---|---|
| | ELISA | Kon | Kdis | KD | EC₅₀ | |
| | EC₅₀ (nM) | (1/Ms) | (1/s) | (nM) | μg/ml | nM |
| IgM-06 | 0.10 | 2.45E5 | <1E−7 | <0.001 | 0.06 | 0.067 |
| IgG-06 | 1.76 | 1.82E5 | 1.36E−4 | 0.75 | 0.15 | 1.0 |
| Fold | 17.6 | 1.35 | <7.35E−4 | >750 | 2.5 | 14.9 |
| change | | | | | | |

TABLE 4
Binding and Neutralization Characteristics of CoV2-14 Antibodies

| | | Avidity to spike | | | | |
|---|---|---|---|---|---|---|
| | ELISA | Kon | Kdis | KD | EC₅₀ | |
| | EC₅₀ (nM) | (1/Ms) | (1/s) | (nM) | μg/ml | nM |
| IgM-14 | 0.046 | 3.35E6 | 3.4E−4 | 0.102 | 0.01 | 0.011 |
| IgG-14 | 6.22 | 9.33E5 | 1.3E−3 | 1.39 | 0.39 | 2.6 |
| Fold | 135 | 3.59 | 0.26 | 13.6 | 39 | 236 |
| change | | | | | | |

### Example 5: CoV2-06 and CoV2-14 IgM Antibodies Show Enhanced Blocking of the ACE2-SARS-CoV2 RBD Interaction

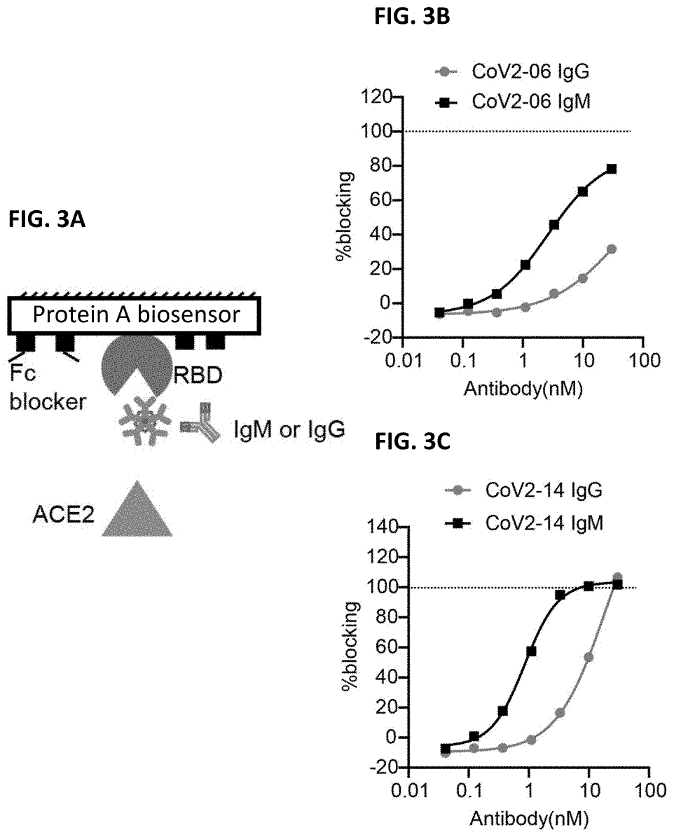

The ability of the disclosed antibodies to block the interaction between the SARS-CoV-2 RBD and the human ACE2 protein was performed using a bio-layer interferometry (BLI)-based competition assay, as follows. A schematic of the assay is shown in FIG. 3A. The wild type RBD protein (3 μg/ml), generated as previously described (Ku, Z, et al., Nature Comm. 12:469 doi:10.1038/s41467-020-20789-7, 2021), was captured on the protein A biosensor for 300s. Then, the sensors were blocked by a control Fc protein (100 μg/ml) for 200s to occupy the free protein A on the sensor. The serially diluted antibodies (0.041˜30 nM) were then incubated with the sensors for 200s to allow antibody and RBD binding. The irrelevant isotype antibodies (30 nM) were used as controls. After 10s of baseline in kinetics buffer, the sensors were dipped into the ACE2 solution (10 μg/ml) for 200s to record the response signal. For analysis of the half-maximal effective concentration (EC50) of the ACE2 blocking, the ACE2 response values were normalized to the starting points. The blocking percentages at each concentration were calculated as: ((normalized ACE2 response of isotype antibody −normalized ACE2 response of tested antibody)/normalized ACE2 response of isotype antibody)*100. The dose-blocking curves were plotted and the blocking EC₅₀ values were calculate by the GraphPad prism 8 Software.

The results are shown in FIG. 3B (CoV2-06 IgM and IgG) and FIG. 3C (CoV2-14 IgM and IgG). Both IgMs had improved blocking activities against RBD and ACE2 interactions compared with IgGs. CoV2-14 IgM showed full blocking, but neither CoV2-06 IgM nor CoV2-06 IgG fully blocked ACE2 binding, even at the highest concentrations tested.

### Example 6: Potency of Multimeric SARS-CoV-2 Antibodies Against SARS-CoV-2 Escape Mutants

K444R and E484A variants corresponding to SEQ ID NO: 102 were recently identified as neutralization-resistant RBD mutations associated with CoV2-06 IgG and CoV2-14 IgG, respectively (Ku, Z, et al., Nature Comm. 12:469 doi:10.1038/s41467-020-20789-7, 2021). Briefly, escape mutants more resistant to neutralization mediated by CoV2-06 IgG and CoV2-14 IgG were generated by incubating SARS-CoV-2 (Isolate: USA-WA1/2020) containing a fluorescence protein mNeonGreen (SARS-CoV-2 mNG) with sequentially increasing concentrations of CoV2-06 IgG or CoV2-14 IgG (10 to 200 μg/mL), followed by 3 rounds of replication selection on Vero E6 cells (1 round for 3-4 days and 2 rounds for 2-3 days). CoV2-06 IgM and IgG and CoV2-14 IgM and IgG were compared for their coverages of these neutralization-resistant RBD mutations, as follows. First, the mutations associated with viral escape isolated from CoV2-06 IgG (K444R mutation) and CoV2-14 IgG (E484 mutation) were characterized in neutralization assays. We constructed three mNeonGreen (mNG) SARS-CoV-2 viruses that had the K444R, E484A, or K444R+E484A mutations, respectively, by the methods described in Example 3 and as described in Muruato et al. (Nat Commun 11, 4059 doi: 10.1038/s41467-020-17892-0 (2020)). Both CoV2-06 IgM and IgG effectively neutralized the E484A virus (FIG. 4B), but not the K444R virus (FIG. 4A) or K444R+E484A virus (FIG. 4C). CoV2-14 IgG effectively neutralized the K444R virus (FIG. 4D) and only weakly neutralized the E484A virus and K444R+E484A viruses (FIGS. 4E and 4F). Importantly, CoV2-14 IgM effectively neutralized all three mutant viruses including the E484A virus and the K444R+E484A virus (FIGS. 4D-4F), which were relatively resistant to CoV2-14 IgG (FIGS. 4D-4F) and an CoV2-06 IgG+CoV2-14 IgG cocktail (data not shown). The neutralization EC₅₀s of CoV2-14 IgM against the E484A virus and the K444R+E484A virus were 0.064 μg/ml and 0.055 μg/ml, respectively, which were comparable to the neutralization EC₅₀ (0.015 μg/ml) against the wild type (WT) virus. We also used the K444R, E484A, and K444R+E484A RBD mutant proteins to compare the binding activities and the ACE2 blocking activities of CoV2-06 IgM and IgG and CoV2-14 IgM and IgG, generally as described in Examples 4 and 5. Consistently, both IgMs showed stronger RBD binding and ACE2 blocking activities than IgGs. These data, along with the neutralization data are summarized in Table 5.

TABLE 5
Binding and Neutralization Characteristics of CoV2-06 IgM and IgG and CoV2-14 IgM
and IgG against Escape Mutants

| | | | CoV2-06 | CoV2-14 | CoV2-14 |
|---|---|---|---|---|---|
| | RBD mutations | CoV2-06 G | IgM | IgG | IgM |
| Neutralization | WT | 0.22 | 0.059 | 0.39 | 0.015 |
| EC₅₀ (μg/ml) | K444R | >200 | >200 | 0.95 | 0.012 |
| | E484A | 0.78 | 0.046 | 150 | 0.064 |
| | K444R + E484A | >200 | >16.5 | 107.2 | 0.055 |
| Binding | WT | <0.001 | <0.001 | 0.82 | <0.001 |
| KD (nM) | K444R | 1.65 | 0.176 | ND | ND |
| | E484A | ND | ND | 1.35 | 0.036 |
| | K444R + E484A | 40 | 0.33 | 1.42 | 0.076 |
| ACE2 | WT | 44.5 | 2.63 | 18.58 | 0.86 |
| blocking | K444R | 13.4 | 2.49 | ND | ND |
| EC50 (nM) | E484A | ND | ND | 48.98 | 2.21 |
| | K444R + E484A | >30 | 9.36 | 55.46 | 2.51 |

When the same approach was used in an effort to isolate escape mutants to CoV2-14 IgM, no escape mutants were recovered. An alternative approach was therefore explored where SARS-CoV-2-mNG was initially incubated with a lower concentration of CoV2-14 IgM (0.5 μg/mL, 50×EC₅₀), and the first round of selection was extended to 6 days. Supernatant from this culture was then successively weaned by incubation with increasing concentrations of CoV2-14 IgM (up to 50 μg/mL, or 5,000×EC₅₀) over multiple rounds of selection lasting 5 days for the three earlier rounds of selection and 2 days for the late rounds of selection.

After 7 rounds of selection under these conditions, three CoV2-14 IgM escape mutant viruses were recovered from four independent selections (one selection failed to recover viruses and was terminated after the second round). Two of these viruses had a single G476D mutation and one had a single F486S mutation (mutations relative to SEQ ID NO: 102). The latter also included mixed residues at position 261 corresponding to SEQ ID NO: 102 (wild-type and G261R), but this position is well outside the RBD region. Both the G476D and F486S escape mutants are located adjacent to residues critical for CoV2-14 IgM binding and have EC₅₀ values >50 μg/mL (data not shown).

Since the above mutations map to the RBD:hACE2 binding interface, the relative fitness and resistance of SARS-CoV-2 viruses containing these mutations will be evaluated via neutralization, binding, and ACE2 blocking studies.

In addition, CoV2-14 IgM and CoV2-14 IgG were tested for binding and ACE2 blocking against a panel of nineteen RBD mutants described in the literature to characterize the binding and ACE2 blocking activities. RBD proteins that contain amino acid mutations, including N439K, S477N, N501Y, E484K+N501Y, K417N+E484K+N501Y, E484K, F490S, Q493R, S494P, K417E, Y453F, L455F, G476S, F486V, Q493K, K444Q, V445A and G446V (corresponding to SEQ ID NO: 102), were generated by overlap PCR using specific primers. The N439K and S477N are prevalent RBD mutations in circulation and are associated with resistance to several neutralizing IgG mAbs (Thomson, E. C., et al. Cell 184(5):1171-1187.e20. doi: 10.1016/j.cell.2021.01.037 (2021); Liu, Z., et al. Cell Host Microb. 29:477-488 doi: 10.1016/j.chom.2021.01.014 (2021); Weisblum, Y., et al., eLife 9:e61312 doi: 10.7554/eLife.61312 (Oct. 28, 2020)).

Other RBD mutations are associated with resistance to three approved mAbs, Bamlanivimab (E484K, F490S, Q493R, S494P), Casirivimab (REGN-10933, K417E, Y45F, L455F, G476S, F486V, Q493K) and Imdevimab (REGN-10987, K444Q, V445A, G446V) (Fact Sheet for Health Care Providers Emergency Use Authorization (EUA) of Bamlanivimab, (2020); Fact Sheet for Health Care Providers Emergency Use Authorization (EUA) Of Casirivimab and Imdevimab, (2020), both available at fda.gov (visited Jan. 25, 2021). The K417N, E484K and N501Y mutations are associated with the recently emerged B.1.351 lineage in South Africa and severely compromise the neutralizing efficacy of multiple class I and class II mAbs, including several clinical-stage candidates (Wibmer, C K et al., Nature Med.:27:622-625, doi: 10.1038/s41591-021-01285-x (2021)). ACE2 blocking and ELISA binding assays were carried out generally as described in Examples 4 and 5. The results are shown in Table 6. For all these RBD mutants, CoV2-14 IgM showed superior binding (3.72-1,990 fold) and ACE2 blocking (5.9-24.1 fold) activities relative to CoV2-14 IgG. Notably, the ACE2 blocking EC50s of CoV2-14 IgM against the N501Y, E484K+N501Y and K417N+E484K+N501Y mutants were comparable to the WT virus (1.665 nM, 0.681 nM, and 0.368 nM vs. 0.86 nM).

TABLE 6
Activity of CoV2-14 IgM and IgG Against Mutant Spike Proteins

| | | Binding KD (nM) | | ACE2 Blocking EC₅₀ (nM) | |
|---|---|---|---|---|---|
| Mutant | RBD | CoV2-14 | CoV2-14 | CoV2-14 | CoV2-14 |
| Type | Mutations | IgM | IgG | IgM | IgG |
| CoV2-14 IgG | E484A | 0.036 | 1.35 | 2.21 | 48.98 |
| resistant | F486S | 0.16 | 7.01 | 0.89 | 14.98 |
| Natural | N439K | <0.001 | 1.7 | 1.44 | 20.84 |
| escape | S477N | <0.001 | 1.99 | 1.76 | 11.28 |
| variants | N501Y | <0.001 | 0.38 | 1.67 | 12.07 |
| | K417N | <0.001 | 0.17 | 0.85 | 25.01 |
| | E484K, | 0.037 | 0.72 | 0.68 | 292.70 |
| | N501Y | | | | |
| | K417N, | 0.10 | 0.60 | 0.37 | 46.41 |
| | E484K, | | | | |
| | N501Y | | | | |
| Bamlanivimab | E484K | 0.078 | 0.29 | 3.45 | >30 |
| resistant | F490S | <0.001 | 0.45 | 1.54 | 10.60 |
| | Q493R | <0.001 | 0.56 | 1.86 | 12.23 |
| | S494P | <0.001 | 0.46 | 1.68 | 11.78 |
| Casirivimab | K417E | <0.001 | 0.11 | 1.03 | 15.67 |
| resistant | Y453F | <0.001 | 0.60 | 1.56 | 31.66 |
| | L455F | <0.001 | 0.65 | 1.45 | 21.41 |
| | G476S | <0.001 | 0.69 | 1.46 | 33.87 |
| | F486V | <0.001 | 0.26 | 1.65 | 42.65 |
| | Q493K | <0.001 | 0.40 | 1.79 | 25.31 |
| Imdevimab | K444Q | <0.001 | 0.45 | 1.43 | 18.60 |
| resistant | V445A | <0.001 | 0.50 | 1.41 | 33.99 |
| | G446V | <0.001 | 0.16 | 0.85 | 4.99 |

### Example 7: Evaluation of CoV2-06 IgM and CoV2-14 IgM in Therapeutic and Prophylactic Animal Models

We evaluated the protective efficacy of CoV2-06 IgM and CoV2-14 IgM in a mouse model of SARS-CoV-2 infection as follows. The animal study was carried out in accordance with the recommendations for care and use of animals by the Office of Laboratory Animal Welfare, National Institutes of Health, and the protocol was approved by the Institutional Animal Care and Use Committee (IACUC) of University of Texas Medical Branch (UTMB). Ten- to twelve-week-old female BALB/c mice were purchased from Charles River Laboratories and maintained in SealsafeHEPA-filtered air in/out units. A previously described mouse infection model (Ku, Z, et al., Nature Comm. 12:469 doi:10.1038/s41467-020-20789-7, 2021) was used to evaluate antibody protections. For challenge, animals were anesthetized with isoflurane and infected intranasally (IN) with 10⁴ plaque-forming unit (pfu) of mouse-adapted SARS-CoV-2 (SARS-CoV-2 CMA-4, which has an N501Y mutation in the RBD) in 50 μl of phosphate-buffered saline (PBS). Antibodies were intranasally delivered at 6 hours before or 6 hours after viral infection. Two days after infection, lung samples of infected mice were harvested and homogenized in 1 ml PBS for analysis of infectious virus by plaque assay.

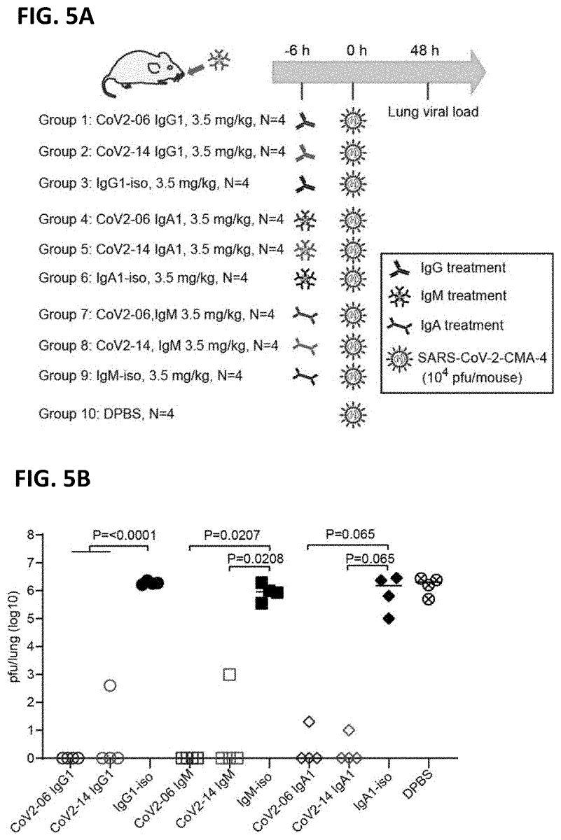

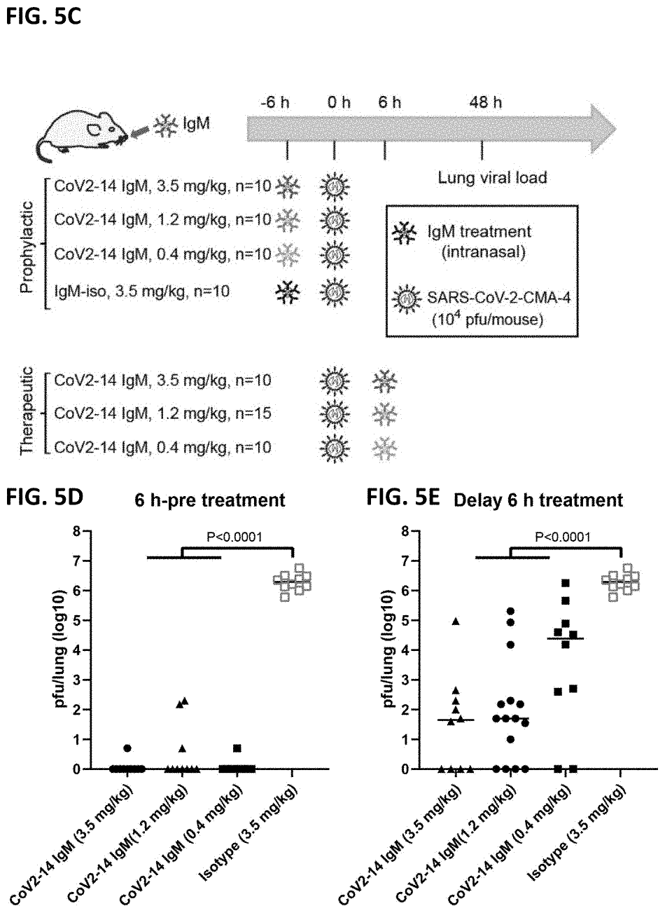

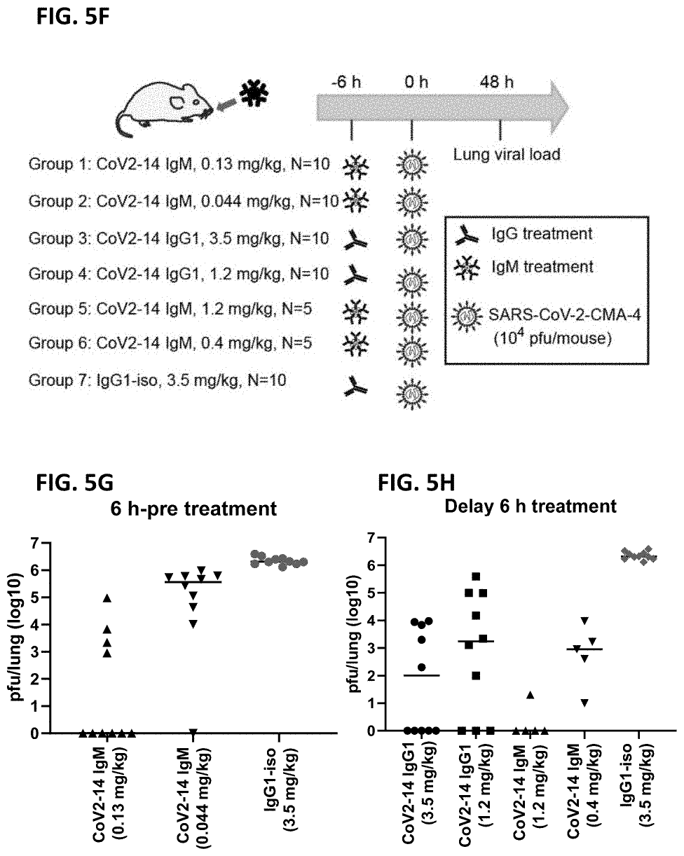

Intraperitoneal injections of CoV2-06 IgG and CoV2-14 IgG confer partial protections at the dose of 5 mg/kg (Ku, Z, et al., Nature Comm. 12:469 doi:10.1038/s41467-020-20789-7, 2021). The prophylactic effects of CoV2-06 IgM, IgA, and IgG and CoV2-14 IgM, IgA, and IgG by intranasal delivery of these antibodies at the dose of 3.5 mg/kg was tested according to the scheme shown in FIG. 5A. The results are shown in FIG. 5B. CoV2-06 IgG and CoV2-06 IgM treatment reduced lung viral loads to undetectable levels for all the mice. CoV2-06 IgA treatment reduced lung viral loads to undetectable levels in three of the four mice and to 1-2 log 10 pfu levels for the remaining one mouse. CoV2-14 IgG and CoV2-14 IgM treatment reduced lung viral loads to undetectable levels in three of the four mice and to 2-3 log 10 pfu levels for the remaining one mouse in each group. CoV2-14 IgA treatment reduced lung viral loads to undetectable levels in three of the four mice and to 1-2 log 10 pfu levels for the remaining one mouse.

In a second study, CoV2-14 IgM was evaluated for its prophylactic and therapeutic efficacies at three dose levels (3.5 mg/kg, 1.2 mg/kg and 0.4 mg/kg), according to the schematic shown in FIG. 5C. The results are shown in FIG. 5D (prophylactic treatment) and FIG. 5E (therapeutic treatment). In the prophylactic treatment model, all dose groups showed almost full reductions of lung viral loads, whereas high virus titers were observed in the animals treated with the isotype control antibody. In the therapeutic treatment model, the median lung viral loads were significantly reduced relative to the isotype control in a dose dependent manner for the groups dosed 3.5 mg/kg, 1.2 mg/kg, or 0.4 mg/kg of CoV2-14 IgM. These data indicate effective protection against SARS-CoV-2 infection both prophylactically and therapeutically by intranasal delivery of CoV2-14 IgM.

In a third study, prophylactic treatment with CoV2-14 IgM was tested according to the schematic shown in FIG. 5F, which includes lower doses than previously tested: 0.13 mg/kg, and 0.044 mg/kg, and in a therapeutic model comparing CoV2-14 IgM (1.2 mg/kg and 0.4 mg/kg) to CoV2-14 IgG (1.2 mg/kg and 0.4 mg/kg). The results are shown in FIG. 5G and FIG. 5H, respectively (the same IgG isotype control is shown in both panels). CoV2-14 IgM at 1.2 mg/kg showed at least 100-fold improvement in protection over CoV2-14 IgG at 1.2 mg/kg, where CoV2-14 IgM at 0.4 mg/kg showed comparable protection to CoV2-14 IgG at 1.2 mg/kg.

### Example 8: Biodistribution of CoV2-14 IgM Following Intranasal Delivery

The biodistribution of CoV2-14 IgM following intranasal (IN) administration was evaluated as follows. CoV2-14 IgM was labeled with Alexa Fluor 750 (AF750) dye for near-infrared fluorescence (NIRF) imaging. Briefly, CoV2-14 IgM (2 mg/ml) was reacted with Alexa Fluor 750 succinimidyl ester (in DMSO and 0.1 M sodium phosphate buffer, pH 8.3) using the molar ratios of 1:10 protein to fluorescent probe at room temperature for 1 h. Unreacted dyes were removed by dialysis and the labeled antibody was washed and concentrated with an Amicon ultra centrifugal filter unit. All procedures were done under dimmed light. CD-1 mice (6-8 weeks, female, Charles River Laboratories, Wilmington, Mass.) were anesthetized by inhalation of 2% isoflurane and placed in a supine position.

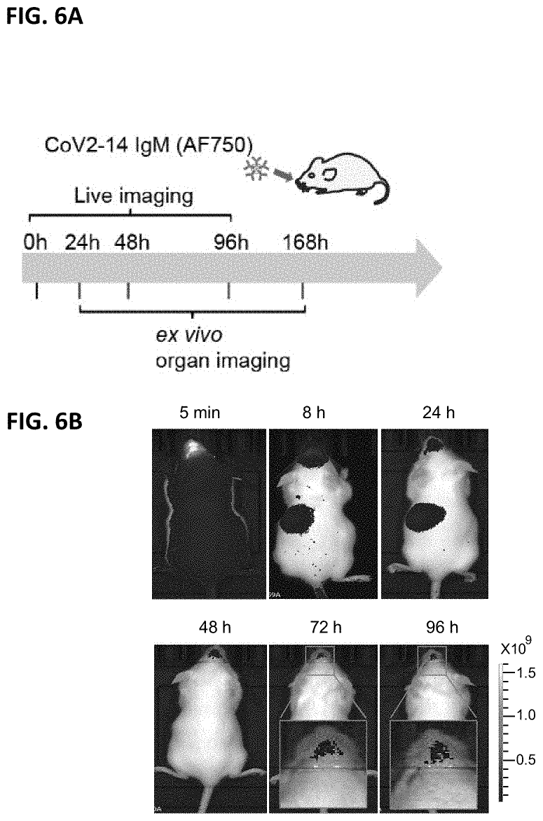

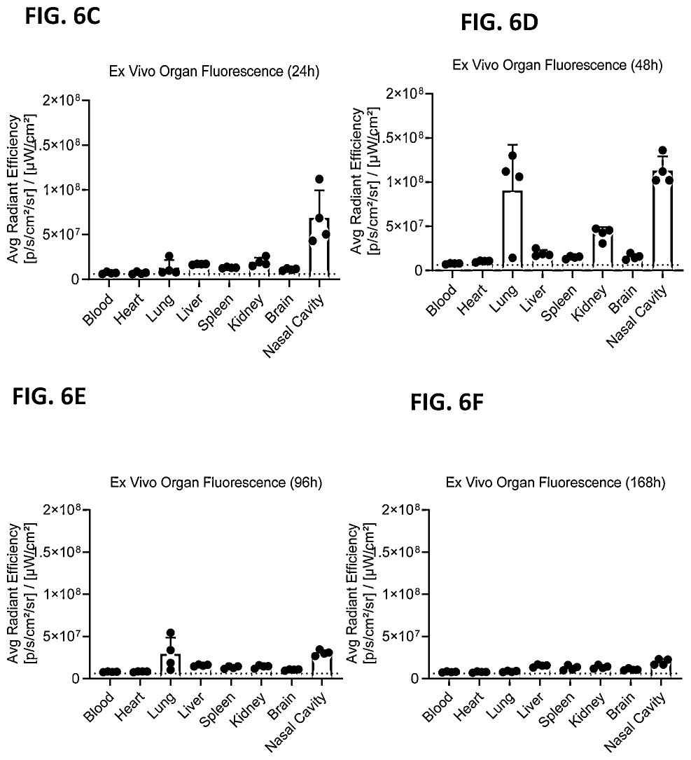

Imaging was carried out according to the scheme shown in FIG. 6A. Briefly, the Alexa Fluor 750-labeled CoV2-14 IgM antibody was administered intranasally to both nostrils of the mice using a fine pipet tip (40 μl total) to achieve the final antibody dose of 1.2 mg/kg. The mice were imaged at predetermined time points post administration (fluorescence Ex=740 nm, Em=780 nm, n=4 mice in each group) using an IVIS Lumina XRMS Imager (PerkinElmer, Waltham, Mass.). Images were processed using Living Image software (PerkinElmer) and the same fluorescence threshold were applied for group comparison. The mouse images at various time points are shown in FIG. 6B. After a single dose, the fluorescence signals enriched in nasal cavities and lasted for up to 96 hours, indicating long-term retention of CoV2-14 IgM in the murine nasal cavities. Ex vivo imaging of different organs showed the enrichment of IgM-14 in nasal cavities at 24h (FIG. 6C) and nasal cavities and lungs at 48h (FIG. 6D), 96h (FIG. 6E), and 168h (FIG. 6F) after antibody exposure. Nasal epithelium is the dominant initial site for SARS-CoV-2 respiratory tract infection, followed by aspiration of virus into the lung (Y. J. Hou et al., Cell 182, 429 (Jul. 23, 2020)). Therefore, intranasally administered IgM-14 can mask the airway to confer protection against SARS-CoV-2 respiratory infection.

### Example 9: CoV2-14 Neutralizes SARS-CoV-2 Viruses with Emerging Spike Mutations

SARS-CoV-2 spike proteins with mutations corresponding to the emerging variants B.1.1.7 or Alpha, B.1.351 or Beta, P.1 or Gamma, B.1.617.2 or Delta, Delta variant Ay.4.2, B.1.525 or Eta, B.1.526 or Iota, B.1.617.1 or Kappa, C37 or Lambda, B.1.621 or Mu, or B.1.1.529 or Omicron lineages were introduced into the SARS-CoV-2 clinical strain USA-WA1/2020 using a PCR-based mutagenesis approach according to the protocol as reported previously (Xie, X. et al., Nature Protocols doi: 10.1038/s41596-021-00491-8 (2021)). The spike protein mutations were prepared on the genetic background of an infectious cDNA clone derived from clinical strain USA-WA1/2020 (Xie, X., et al., Cell Host Microbe 27:841-8 (2020)). The full-length infectious cDNAs were ligated and used as templates to in vitro transcribe full-length viral RNAs. Viruses were recovered from Vero E6 cells electroporated with in vitro transcribed RNAs.

The ability of CoV2-14 IgG and IgM antibodies to neutralize of the SARS-CoV-2 variant viruses carrying the spike protein mutations noted above were tested one or more times using a plaque reduction neutralization test (PRNT), as follows. Briefly, the antibodies were serially diluted in culture medium and incubated with 100 plaque-forming units of WT or mutant viruses at 37° C. for 1 h, after which the antibody-virus mixtures were inoculated onto Vero E6 cell monolayer in six-well plates. After 1 h of infection at 37° C., 2 ml of 2% SeaPlaque agar (Lonza) in DMEM containing 2% FBS and 1% penicillin-streptomycin was added to the cells. After 2 d of incubation, 2 ml of 2% SeaPlaque agar in DMEM containing 2% FBS, 1% penicillin-streptomycin and 0.01% neutral red (Sigma-Aldrich) were added on top of the first layer. After another 16 h of incubation at 37° C., plaque numbers were counted. The antibody concentration and plaque reduction ratios were plotted and the EC₅₀ titers were calculated. All neutralization assays were conducted at the Biosafety Level-3 facility with the approval from the Institutional Biosafety Committee at the University of Texas Medical Branch. The results are shown in FIGS. 7C-7J and in Tables 7 and 8.

In another experiment the ability of CoV2-14 IgG and IgM antibodies to neutralize SARS-CoV-2 variant viruses carrying the Omicron spike protein were compared to neutralization potency of an IgG antibody carrying the VH and VL regions of Sotrovimab (S309, Pinto, D., et al., Nature 583:290-295 (2020)), one of the few antibodies currently approved for emergency use in the United States that neutralizes the Omicron variant. See Takashita, N., et al., New Eng. J. Med, DOI: 10.1056/NEJMc2119407 (2022). The comparative results are shown in Table 9. These results show that the potency of CoV2-14-IgM against the Omicron variant is as good or better than that of 5309.

TABLE 7
Neutralization of Emerging Spike Variants

| | Neutralization EC₅₀ (μg/ml) | | | |
|---|---|---|---|---|
| | | B.1.1.7 Alpha- | P.1 Gamma- | B.1.351 Beta- |
| | US-WA1 | Spike | Spike | Spike |
| CoV2-14 IgG1 | 0.51 | 0.27 | 12.59 | 11.06 |
| CoV2-14 IgM | 0.011 | 0.006 | 0.023 | 0.031 |
| Fold-Increase in | 46 | 45 | 547 | 356 |
| potency of IgM | | | | |
| over IgG | | | | |

The EC₅₀ values for CoV2-14 IgM remained relatively consistent between the wild-type and variant viruses, while CoV2-14 IgG lost more than two logs of potency against the Gamma and Beta variants.

TABLE 8
Neutralization of Emerging Spike Variants

| | | | Fold-Increase in |
|---|---|---|---|
| SARS-CoV-2 Spike | Neutralization EC₅₀ (μg/ml) | | potency of IgM |
| Protein | CoV2-14 IgG1 | CoV2-14 IgM | over IgG |
| US-WA1 (Wild-Type) | 0.32 | 0.0052 | 61 |
| B.1.1.7 Alpha | 0.27 | 0.0060 | 45 |
| B.1.351 Beta | 13 | 0.0078 | 1700 |
| P.1 Gamma | 13 | 0.023 | 560 |
| B.1.617.2 Delta | 0.57 | 0.0020 | 290 |
| C.37 Lambda | 0.26 | 0.0035 | 75 |
| B.1.621 Mu | 25 | 0.0028 | 8800 |
| B.1.1.529 Omicron | 270 | 0.38 | 710 |
| B.1.525 Eta | 7.0 | 0.011 | 650 |
| B.1.617.1 Kappa | 10 | 0.0047 | 2100 |
| B.1.526 Iota | 170 | 0.032 | 5300 |
| AY.4.2 (Delta | 0.44 | 0.0020 | 220 |
| subvariant) | | | |
| B.1.618 | 23 | 0.0043 | 5300 |

TABLE 9
Comparative Neutralization of Omicron Spike Variant

| | Neutralization EC₅₀ (μg/ml) | |
|---|---|---|
| | USA-WA1/2020- | |
| | Spike | Omicron-Spike |
| CoV2-14 IgG1 | 0.60 | 88.31 |
| CoV2-15 IgM | 0.0046 | 0.52 |
| S309 IgG1 | 0.17 | 0.73 |

### Example 10: Evaluation of Viral RNA Reduction by CoV2-14 IgM in Therapeutic and Prophylactic Animal Models

CoV2-14 IgM was evaluated for its prophylactic and therapeutic efficacies at three dose levels (3.5 mg/kg, 1.2 mg/kg and 0.4 mg/kg), as described in Example 7 and according to the schematic shown in FIG. 5C, except lung samples were not analyzed using plaque assay and were instead assayed to determine viral RNA load as described below, and the experiment included an additional control group of 5 uninfected mice to determine the lower limit of detection (LLOD).

Two days after infection, lung samples of infected mice were harvested and homogenized in 1 ml PBS. The homogenates were clarified by centrifugation at 15000 rpm for 5 min. The supernatants were collected for analysis. Quantitative RT-PCR assay was used for measuring viral RNA (Nucleocapsid gene) titers in the lung. Briefly, the clarified tissue homogenates were mixed with a five-fold excess of TRIzol LS Reagent (Thermo Fisher Scientific, Cat #10296010). Total RNA was extracted according to the manufacturer's instructions. The extracted RNA was finally dissolved in 40 μl nuclease-free water. Two microliters of RNA samples were used for quantitative RT-PCR assays using the iTaq SYBR Green one-step kit (Bio-Rad) on the QuantStudio Real-Time PCR systems with fast 96-well module (Thermo Fisher Scientific). The quantification of viral RNA was determined by a standard curve method using an RNA standard (in vitro transcribed 3,839 bp RNA at the nucleotide positions from 26,044 to 29,883 of SARS-CoV-2 genome) and the primers 2019-nCoV N2-F (5′-TTA CAA ACA TTG GCC GCA AA-3′) (SEQ ID NO: 103) and 2019-nCoV N2-R (5′-GCG CGA CAT TCC GAA GAA-3′) (SEQ ID NO: 104).

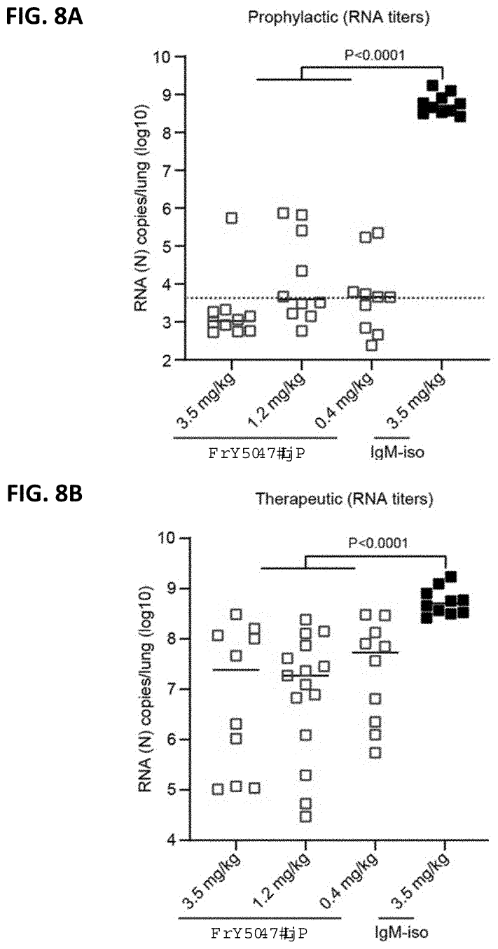

The results are shown in FIG. 8A (prophylactic treatment) and FIG. 8B (therapeutic treatment). The LLOD is shown by a dotted line on both figures. In the prophylactic treatment model, all dose groups showed almost full reductions of lung viral RNA loads, i.e., viral RNA loads at or near the LLOD, whereas high lung viral RNA loads were observed in the animals treated with the isotype control antibody. In the therapeutic treatment model, the median lung viral loads were significantly reduced relative to the isotype control in a dose dependent manner for the groups dosed with 3.5 mg/kg, 1.2 mg/kg, or 0.4 mg/kg of CoV2-14 IgM. These data, like the data discussed in Example 7, indicate effective protection against SARS-CoV-2 infection both prophylactically and therapeutically by intranasal delivery of CoV2-14 IgM using an alternative method to detect viral load.

### Example 11: Evaluation of CoV2-14 IgM in Therapeutic and Prophylactic Animal Models of SARS-CoV-2 Viruses with Emerging Spike Mutations

CoV2-14 IgM and CoV2-14 IgG were evaluated for their therapeutic efficacies against SARS-CoV-2 viruses with emerging spike mutations, specifically SARS-CoV-2 spike protein mutations corresponding to the emerging variants P.1 or Gamma and B.1.351 or Beta. CoV2-14 IgM and CoV2-14 IgG were evaluated at two dose levels (3.5 mg/kg and 1.2 mg/kg), as described in Example 7 and according to the therapeutic portion of schematic shown in FIG. 5C. Lung samples were analyzed using the plaque assay described in Example 7.

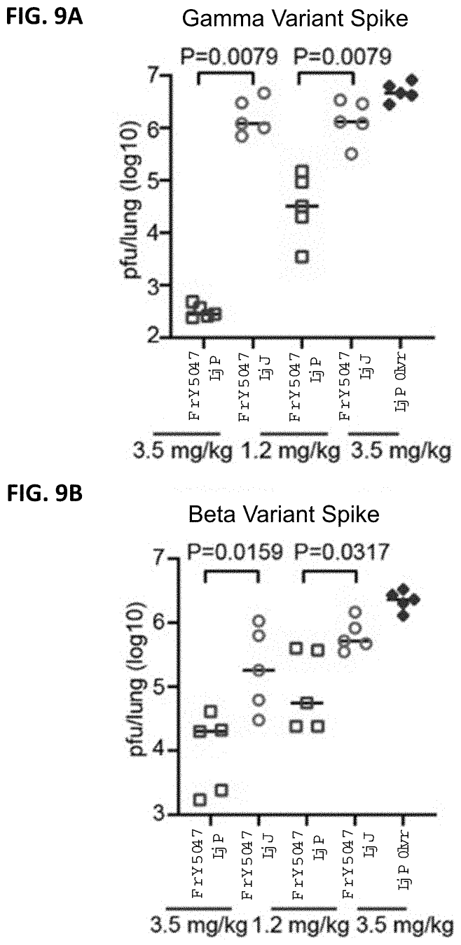

The results for the Gamma variant are shown in FIG. 9A, and the results for the Beta variant are shown in FIG. 9B. Administration of CoV2-14 IgM at either dose resulted in significantly reduced lung viral load of both the Gamma and Beta SARS-CoV-2 variants compared to administration of CoV2-14 IgG.

These data indicate effective protection against emerging variants of SARS-CoV-2 therapeutically by intranasal delivery of CoV2-14 IgM, even when the CoV2-14 IgG has minimal or no efficacy against said variants.

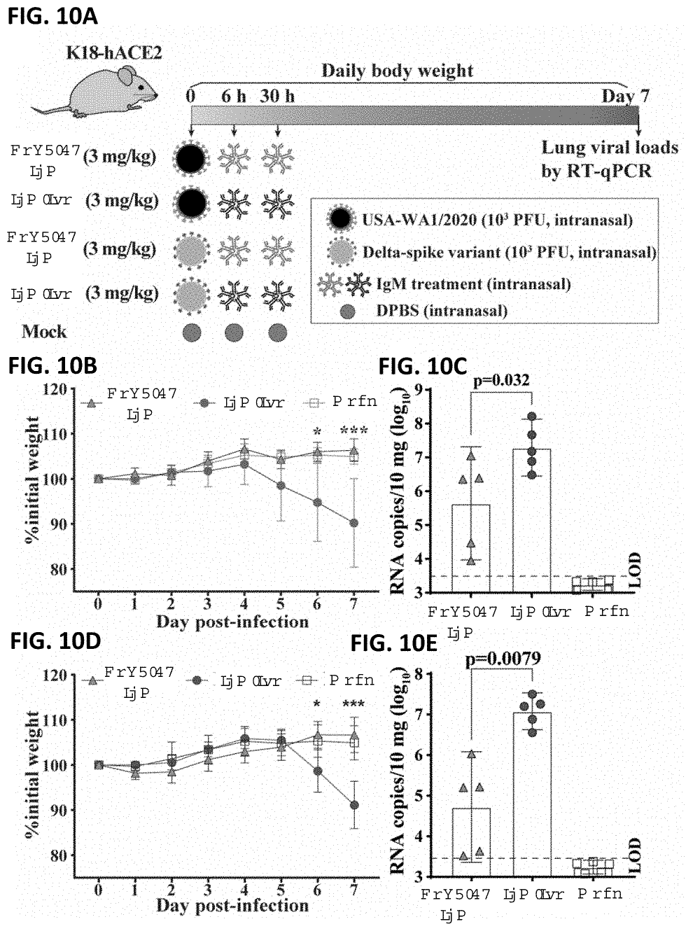

CoV2-14 IgM was further evaluated for its therapeutic efficacy against SARS-CoV-2 carrying the Delta spike mutations. CoV2-14 IgM (3 mg/kg) was evaluated in a therapeutic model using K18-hACE2 transgenic mice against wild type (USA-WA1/2020) SARS CoV-2 and recombinant USA-WA1/202 carrying the spike protein of the Delta variant, prepared as reported previously (Xie, X. et al., Nature Protocols doi: 10.1038/s41596-021-00491-8 (2021)), according to the schematic shown in FIG. 10A. Briefly, animals were inoculated intranasally with 10³ pfu of USA-WA1/2020, the recombinant virus carrying the Delta spike protein, or mock infected at day zero, and were then treated intranasally with CoV2-14 IgM or an isotype control (3 mg/kg) at 6 hours and 30 hours post-infection. Animals were evaluated for weight loss for seven days, and at day seven the animals were euthanized and lungs were harvested for quantitation of viral RNA as described in Example 10.

The results for wild-type SARS-CoV-2 are shown are shown in FIGS. 10B and 10C, and the results for the recombinant virus carrying the Delta variant spike protein are shown in FIGS. 10D and 10E. Administration of CoV2-14 IgM following both wild-type and recombinant Delta variant infection resulted in significantly reduced weight loss and significantly reduced lung viral load over the isotype control-treated animals.

### Example 12: Evaluation of Receptor-Independent Replication in Immune Cells

The potential for CoV2-14 IgM to mediate receptor-independent replication in immune cells (antibody-dependent enhancement, or ADE) was evaluated as follows. Peripheral blood mononuclear cells (PBMCs) were selected for these studies because several immune subsets express mu receptors whereas typical human immune cell lines like THP-1 cells do not (Bosshart, H., and M. Heinzelmann, Ann Transl Med 4:438 (2016)). PBMCs from 3 different donors were incubated with a replication competent reporter virus (SARS-CoV-2 containing a luciferase gene) at MOIs ranging from 5 to 10 without and with CoV2-14 IgM or CoV2-14 IgG at concentrations ranging from 0.005 to 10×EC50. Luciferase signals were then measured at two different time points post infection (24 h and 48 h). No luciferase signals were detected in these cultures, indicating that little or no virus replication had occurred.

TABLE 10
Antibody VH, VL Sequences (Kabat CDRs Underlined)

| | SEQ | | SEQ | |
|---|---|---|---|---|
| | ID NO | VH | ID NO | VL |
| CoV2-06 | 14 | QVELQESGPGLVKPSGTLSLTCAVSGGSISSNNWWT | 18 | QAVVTQPASVSGSPGQSITISCTGTSSDVGGYNYVSW |
| (Kabat CDRs | | WVRQPPGKGLEWIGEIHHSGGTNYNPSLKSRVTMS | | YQQHPGKAPKLMIYDVSNRPSGVSNRFSGSKSGNTAS |
| underlined) | | VDKSKNQFSLNLYSVTAADTAVYYCTRDRAGGTYS | | LTISGLQAEDEADYYCSSYTSSSTVVFGGGTKVTVL |
| | | GFDFWGQGTLVTVSS | | |
| CoV2-09 | 22 | QVQLHQWGAGLLKPSETLSRTCAVYGGSFSGYYW | 26 | QSALTQDPSVSVALGQTVRITCQGDSLRGSFASWYRQ |
| (Kabat CDRs | | TWIRQAPGKGLEWIGEINHGGSTRYNPSLESRVSISV | | KPGQAPVLVIFGINNRPSGVPDRFSGSSSGNTASLTITG |
| underlined) | | DTSKKQFSLELRSVTAADTAIYYCARGYDTNWYGD | | AQAEDEADYYCNSRESNSNRILFGGGTKVTVL |
| | | GYNWFDPWDRGTLVTVSS | | |
| CoV2-12 | 30 | QVQLVESGGGVVQPGRSLRLSCVASGFTFSSYAMQ | 34 | DIQMTQSPSSLSASVGDRVTITCRASQSISSYLNWYQQ |
| (Kabat CDRs | | WVRQAPGKGLEYVAVVSDDGNMKFYADSVKGRFT | | KPGKAPKLLIYAASSLQSGVPSRFSGSGSGTDFTLTISS |
| underlined) | | ISRDNSRNTVSLQMNSLGVEDTAVYYCARENYFWS | | LQPEDFATYYCQQSYSTPGYTFGQGTKLEIK |
| | | GSIGGLDYWGQGTLVSVSS | | |
| CoV2-14 | 38 | EVQLQQSGPGLVKPSQTLSLTCAISGDSVSSNSAAW | 42 | QAVVTQPASVSGSPGQSITISCTGTSSDIGAYNYISWY |
| (Kabat CDRs | | NWIRQSPSRGLEWLGRTYYRSKWYNDYAVSVKSRI | | QQHPGKAPKLIIYEVSNRPSGISYRFSGSKSGNTASLTI |
| underlined) | | TINPDTSKNQFSLQLNSVTPEDTAVYYCAREEQQLV | | SGLQAEDEANYYCSSYAGSISFGGGTKVTVL |
| | | HDYYYYGMDVWGQGTMVTVSS | | |
| CoV2-16 | 46 | QVQLHQWGAGLLKPSETLSRTCAVYGGSFSGYYW | 50 | QAVVTQPPSVSGTPGQRVTISCSGSSSSIGSNTVHWYQ |
| (Kabat CDRs | | TWIRQAPGKGLEWIGEINHGGSTRYNPSLESRVSISV | | HLPGTAPKLLMYNNNERPSGVPARFSGSKSGTSASLVI |
| underlined) | | DTSKKQFSLELRSVTAADTAIYYCARGYDTNWYGD | | TGLQADDEAEYYCQSYDTGLSGHVFGSGTELTVL |
| | | GYNWFDPWDRGTLVTVSS | | |
| CoV2-26 | 54 | QVQLVESGAEVKKPGASVKVSCKASGYTFTGYYM | 58 | QSALTQPPSASGTPGQRVTISCSGSSSNIGSNSVNWYQ |
| (Kabat CDRs | | HWVRQAPGQGLEWMGRINPNSGGTNYAQKFQGRV | | QVPGTAPKLLIYANDHRPSGVPDRFSGSKSGTSASLAI |
| underlined) | | TMTRDTSISTAYMELSRLRSDDTAVYYCARGYFDY | | SGLQSEDEADYYCAAWDNSLKGVVFGGGTKVTVL |
| | | WGQGTLVTVSS | | |
| CR3022 | 98 | QMQLVQSGTEVKKPGESLKISCKGSGYGFITYWIG | 99 | DIQLTQSPDSLAVSLGERATINCKSSQSVLYSSINKNYL |
| | | WVRQMPGKGLEWMGIIYPGDSETRYSPSFQGQVTIS | | AWYQQKPGQPPKLLIYWASTRESGVPDRFSGSGSGTD |
| | | ADKSINTAYLQWSSLKASDTAIYYCAGGSGISTPMD | | FTLTISSLQAEDVAVYYCQQYYSTPYTFGQGTKVEIK |
| | | VWGQGTTVTVSS | | |
| CR3014 | 100 | EVQLVESGGGLVQPGGSLRLSCAASGFTFSDHYMD | 101 | DIQMTQSPSSLSASVGDRVTITCRASQSISSYLNWYQQ |
| | | WVRQAPGKGLEWVGRTRNKANSYTTEYAASVKGR | | KPGKAPKLLIYAASSLQSGVPSRFSGSGSGTDFTLTISS |
| | | FTISRDDSKNSLYLQMNSLKTEDTAVYYCARGISPF | | LQPEDFATYYCQQSYSTPPTFGQGTKVEIK |
| | | YFDYWGQGTLVTVSS | | |

TABLE 11
Antibody CDR Sequences

| | CoV2-06 | CoV2-09 | CoV2-12 | CoV2-14 | CoV2-16 | CoV2-26 |
|---|---|---|---|---|---|---|
| Kabat HCDR1 | SNNWWT (15) | GYYWT (23) | SYAMQ (31) | SNSAAWN (39) | GYYWT (47) | GYYMH (55) |
| (SEQ ID NO) | | | | | | |
| Kabat HCDR2 | EIHHSGGTNYNP | EINHGGSTRYNP | VVSDDGNMKFYA | RTYYRSKWYNDY | EINHGGSTRYNP | RINPNSGGTNYA |
| (SEQ ID NO) | SLKS (16) | SLES (24) | DSVKG (32) | AVSVKS (40) | SLES (48) | QKFQG (56) |
| Kabat HCDR3 | DRAGGTYSGFDF | GYDTNWYGDGYN | ENYFWSGSIGGL | EEQQLVHDYYYY | GYDTNWYGDGYN | GYFDY (57) |
| (SEQ ID NO) | (17) | WFDP (25) | DY (33) | GMDV (41) | WFDP (49) | |
| Kabat LCDR1 | TGTSSDVGGYNY | QGDSLRGSFAS | RASQSISSYLN | TGTSSDIGAYNY | SGSSSSIGSNTV | SGSSSNIGSNSV |
| (SEQ ID NO) | VS (19) | (27) | (35) | IS (43) | H (51) | N (59) |
| Kabat LCDR2 | DVSNRPS (20) | GINNRPS (28) | AASSLQS (36) | EVSNRPS (44) | EVSNRPS (52) | ANDHRPS (60) |
| (SEQ ID NO) | | | | | | |
| Kabat LCDR3 | SSYTSSSTVV | NSRESNSNRIL | QQSYSTPGYT | SSYAGSIS | QSYDTGLSGHV | AAWDNSLKGVV |
| (SEQ ID NO) | (21) | (29) | (37) | (45) | (53) | (61) |
| IMGT HCDR1 | GGSISSNNW | GGSFSGYY | GFTFSSYA | GDSVSSNSAA | GGSFSGYY | GYTFTGYY |
| (SEQ ID NO) | (62) | (68) | (74) | (80) | (86) | (92) |
| IMGT HCDR2 | IHHSGGT (63) | INHGGST (69) | VSDDGNMK | TYYRSKWYN | INHGGST (87) | INPNSGGT |
| (SEQ ID NO) | | | (75) | (81) | | (93) |
| IMGT HCDR3 | TRDRAGGTYSGF | ARGYDTNWYGDG | ARENYFWSGSIG | AREEQQLVHDYY | ARGYDTNWYGDG | ARGYFDY (94) |
| (SEQ ID NO) | DF (64) | YNWFDP (70) | GLDY (76) | YYGMDV (82) | YNWFDP (88) | |
| IMGT LCDR1 | SSDVGGYNY | SLRGSF (71) | QSISSY (77) | SSDIGAYNY | SSSIGSNT | SSNIGSNS |
| (SEQ ID NO) | (65) | | | (83) | (89) | (95) |
| IMGT LCDR2 | DVS (66) | GIN (72) | AAS (78) | EVS (84) | NNN (90) | AND (96) |
| (SEQ ID NO) | | | | | | |
| IMGT LCDR3 | SSYTSSSTVV | NSRESNSNRIL | QQSYSTPGYT | SSYAGSIS | QSYDTGLSGHV | AAWDNSLKGVV |
| (SEQ ID NO) | (67) | (73) | (79) | (85) | (91) | (97) |

TABLE 12
Other Sequences in the Disclosure

| SEQ | | |
|---|---|---|
| ID | | |
| NO: | Nickname (source) | Sequence |
| 1 | Human IgM Constant | GSASAPTLFPLVSCENSPSDTSSVAVGCLAQDFLPDSITFSWKYKNNSDISSTRGFPSVLRGGKYAATSQVL |
| | region IMGT allele | LPSKDVMQGTDEHVVCKVQHPNGNKEKNVPLPVIAELPPKVSVFVPPRDGFFGNPRKSKLICQATGFSPRQI |
| | IGHM*03 (GenBank: | QVSWLREGKQVGSGVTTDQVQAEAKESGPTTYKVTSTLTIKESDWLSQSMFTCRVDHRGLTFQQNASSMCVP |
| | pir\|S37768\|) | DQDTAIRVFAIPPSFASIFLTKSTKLTCLVTDLTTYDSVTISWTRQNGEAVKTHTNISESHPNATFSAVGEA |
| | | SICEDDWNSGERFTCTVTHTDLPSPLKQTISRPKGVALHRPDVYLLPPAREQLNLRESATITCLVTGFSPAD |
| | | VFVQWMQRGQPLSPEKYVTSAPMPEPQAPGRYFAHSILTVSEEEWNTGETYTCVVAHEALPNRVTERTVDKS |
| | | TGKPTLYNVSLVMSDTAGTCY |
| 2 | Human IgM Constant | GSASAPTLFPLVSCENSPSDTSSVAVGCLAQDFLPDSITFSWKYKNNSDISSTRGFPSVLRGGKYAATSQVL |
| | region IMGT allele | LPSKDVMQGTDEHVVCKVQHPNGNKEKNVPLPVIAELPPKVSVFVPPRDGFFGNPRKSKLICQATGFSPRQI |
| | IGHM*04 (GenBank: | QVSWLREGKQVGSGVTTDQVQAEAKESGPTTYKVTSTLTIKESDWLGQSMFTCRVDHRGLTFQQNASSMCVP |
| | sp\|P01871.4\|) | DQDTAIRVFAIPPSFASIFLTKSTKLTCLVTDLTTYDSVTISWTRQNGEAVKTHTNISESHPNATFSAVGEA |
| | | SICEDDWNSGERFTCTVTHTDLPSPLKQTISRPKGVALHRPDVYLLPPAREQLNLRESATITCLVTGFSPAD |
| | | VFVQWMQRGQPLSPEKYVTSAPMPEPQAPGRYFAHSILTVSEEEWNTGETYTCVVAHEALPNRVTERTVDKS |
| | | TGKPTLYNVSLVMSDTAGTCY |
| 3 | Human IgA1 heavy chain | ASPTSPKVFPLSLCSTQPDGNVVIACLVQGFFPQEPLSVTWSESGQGVTARNFPPSQDASGDLYTTSSQLTL |
| | constant region, e.g., | PATQCLAGKSVTCHVKHYTNPSQDVTVPCPVPSTPPTPSPSTPPTPSPSCCHPRLSLHRPALEDLLLGSEAN |
| | amino acids 144 to 496 | LTCTLTGLRDASGVTFTWTPSSGKSAVQGPPERDLCGCYSVSSVLPGCAEPWNHGKTFTCTAAYPESKTPLT |
| | of GenBank AIC59035.1 | ATLSKSGNTFRPEVHLLPPPSEELALNELVTLTCLARGFSPKDVLVRWLQGSQELPREKYLTWASRQEPSQG |
| | | TTTFAVTSILRVAAEDWKKGDTFSCMVGHEALPLAFTQKTIDRLAGKPTHVNVSVVMAEVDGTCY |
| 4 | Human IgA2 heavy chain | ASPTSPKVFPLSLDSTPQDGNVVVACLVQGFFPQEPLSVTWSESGQNVTARNFPPSQDASGDLYTTSSQLTL |
| | constant region, e.g., | PATQCPDGKSVTCHVKHYTNSSQDVTVPCRVPPPPPCCHPRLSLHRPALEDLLLGSEANLTCTLTGLRDASG |
| | amino acids 1 to 340 of | ATFTWTPSSGKSAVQGPPERDLCGCYSVSSVLPGCAQPWNHGETFTCTAAHPELKTPLTANITKSGNTFRPE |
| | GenBank P01877.4 | VHLLPPPSEELALNELVTLTCLARGFSPKDVLVRWLQGSQELPREKYLTWASRQEPSQGTTTYAVTSILRVA |
| | | AEDWKKGETFSCMVGHEALPLAFTQKTIDRMAGKPTHINVSVVMAEADGTCY |
| 5 | Precursor Human | MLLFVLTCLLAVFPAISTKSPIFGPEEVNSVEGNSVSITCYYPPTSVNRHTRKYWCRQGARGGCITLISSEG |
| | Secretory Component | YVSSKYAGRANLTNFPENGTFVVNIAQLSQDDSGRYKCGLGINSRGLSFDVSLEVSQGPGLLNDTKVYTVDL |
| | | GRTVTINCPFKTENAQKRKSLYKQIGLYPVLVIDSSGYVNPNYTGRIRLDIQGTGQLLFSVVINQLRLSDAG |
| | | QYLCQAGDDSNSNKKNADLQVLKPEPELVYEDLRGSVTFHCALGPEVANVAKFLCRQSSGENCDVVVNTLGK |
| | | RAPAFEGRILLNPQDKDGSFSVVITGLRKEDAGRYLCGAHSDGQLQEGSPIQAWQLFVNEESTIPRSPTVVK |
| | | GVAGGSVAVLCPYNRKESKSIKYWCLWEGAQNGRCPLLVDSEGWVKAQYEGRLSLLEEPGNGTFTVILNQLT |
| | | SRDAGFYWCLTNGDTLWRTTVEIKIIEGEPNLKVPGNVTAVLGETLKVPCHFPCKFSSYEKYWCKWNNTGCQ |
| | | ALPSQDEGPSKAFVNCDENSRLVSLTLNLVTRADEGWYWCGVKQGHFYGETAAVYVAVEERKAAGSRDVSLA |
| | | KADAAPDEKVLDSGFREIENKAIQDPRLFAEEKAVADTRDQADGSRASVDSGSSEEQGGSSRALVSTLVPLG |
| | | LVLAVGAVAVGVARARHRKNVDRVSIRSYRTDISMSDFENSREFGANDNMGASSITQETSLGGKEEFVATTE |
| | | STTETKEPKKAKRSSKEEAEMAYKDFLLQSSTVAAEAQDGPQEA |
| 6 | Precursor Human J Chain | MKNHLLFWGVLAVFIKAVHVKAQEDERIVLVDNKCKCARITSRIIRSSEDPNEDIVERNIRIIVPLNNRENI |
| | | SDPTSPLRTRFVYHLSDLCKKCDPTEVELDNQIVTATQSNICDEDSATETCYTYDRNKCYTAVVPLVYGGET |
| | | KMVETALTPDACYPD |
| 7 | Mature Human J Chain | QEDERIVLVDNKCKCARITSRIIRSSEDPNEDIVERNIRIIVPLNNRENISDPTSPLRTRFVYHLSDLCKKC |
| | | DPTEVELDNQIVTATQSNICDEDSATETCYTYDRNKCYTAVVPLVYGGETKMVETALTPDACYPD |
| 8 | J Chain Y102A mutation | QEDERIVLVDNKCKCARITSRIIRSSEDPNEDIVERNIRIIVPLNNRENISDPTSPLRTRFVYHLSDLCKKC |
| | | DPTEVELDNQIVTATQSNICDEDSATETCATYDRNKCYTAVVPLVYGGETKMVETALTPDACYPD |
| 9 | “5” Peptide linker | GGGGS |
| 10 | “10” Peptide linker | GGGGSGGGGS |
| 11 | “15” Peptide linker | GGGGSGGGGSGGGGS |
| 12 | “20” Peptide linker | GGGGSGGGGSGGGGSGGGGS |
| 13 | “25” Peptide Linker | GGGGSGGGGSGGGGSGGGGSGGGGS |
| 102 | SARS-CoV-2 Spike (S) | >sp\|P0DTC2\|SPIKE_SARS2 Spike glycoprotein OS = Severe acute respiratory |
| | Protein, UniProt P0DTC2 | syndrome coronavirus 2 OX = 2697049 GN = S PE = 1 SV = 1 |
| | | MFVFLVLLPLVSSQCVNLTTRTQLPPAYTNSFTRGVYYPDKVERSSVLHSTQDLFLPFFSNVTWFHAIHVSG |
| | | TNGTKRFDNPVLPFNDGVYFASTEKSNIIRGWIFGTTLDSKTQSLLIVNNATNVVIKVCEFQFCNDPFLGVY |
| | | YHKNNKSWMESEFRVYSSANNCTFEYVSQPFLMDLEGKQGNFKNLREFVFKNIDGYFKIYSKHTPINLVRDL |
| | | PQGFSALEPLVDLPIGINITRFQTLLALHRSYLTPGDSSSGWTAGAAAYYVGYLQPRTFLLKYNENGTITDA |
| | | VDCALDPLSETKCTLKSFTVEKGIYQTSNFRVQPTESIVRFPNITNLCPFGEVFNATRFASVYAWNRKRISN |
| | | CVADYSVLYNSASFSTFKCYGVSPTKLNDLCFTNVYADSFVIRGDEVRQIAPGQTGKIADYNYKLPDDFTGC |
| | | VIAWNSNNLDSKVGGNYNYLYRLFRKSNLKPFERDISTEIYQAGSTPCNGVEGFNCYFPLQSYGFQPTNGVG |
| | | YQPYRVVVLSFELLHAPATVCGPKKSTNLVKNKCVNFNENGLTGTGVLTESNKKFLPFQQFGRDIADTTDAV |
| | | RDPQTLEILDITPCSFGGVSVITPGTNTSNQVAVLYQDVNCTEVPVAIHADQLTPTWRVYSTGSNVFQTRAG |
| | | CLIGAEHVNNSYECDIPIGAGICASYQTQTNSPRRARSVASQSITAYTMSLGAENSVAYSNNSIAIPTNFTI |
| | | SVTTEILPVSMTKTSVDCTMYICGDSTECSNLLLQYGSFCTQLNRALTGIAVEQDKNTQEVFAQVKQTYKTP |
| | | PIKDFGGFNFSQILPDPSKPSKRSFIEDLLFNKVTLADAGFIKQYGDCLGDIAARDLICAQKFNGLTVLPPL |
| | | LTDEMIAQYTSALLAGTITSGWTFGAGAALQIPFAMQMAYRFNGIGVTQNVLYENQKLIANQFNSAIGKIQD |
| | | SLSSTASALGKLQDVVNQNAQALNTLVKQLSSNFGAISSVLNDILSRLDKVEAEVQIDRLITGRLQSLQTYV |
| | | TQQLIRAAEIRASANLAATKMSECVLGQSKRVDFCGKGYHLMSFPQSAPHGVVFLHVTYVPAQEKNFTTAPA |
| | | ICHDGKAHFPREGVFVSNGTHWFVTQRNFYEPQIITTDNTFVSGNCDVVIGIVNNTVYDPLQPELDSFKEEL |
| | | DKYFKNHTSPDVDLGDISGINASVVNIQKEIDRLNEVAKNLNESLIDLQELGKYEQYIKWPWYIWLGFIAGL |
| | | IAIVMVTIMLCCMTSCCSCLKGCCSCGSCCKFDEDDSEPVLKGVKLHYT |
| 103 | 019-nCoV_N2-F | TTA CAA ACA TTG GCC GCA AA |
| 104 | 2019-nCoV_N2-R | GCG CGA CAT TCC GAA |
| 105 | CoV2-14 IgM Heavy Chain | EVQLQQSGPGLVKPSQTLSLTCAISGDSVSSNSAAWNWIRQSPSRGLEWLGRTYYRSKWYNDYAVSVKSRIT |
| | | INPDTSKNQFSLQLNSVTPEDTAVYYCAREEQQLVHDYYYYGMDVWGQGTMVTVSSGSASAPTLFPLVSCEN |
| | | SPSDTSSVAVGCLAQDFLPDSITFSWKYKNNSDISSTRGFPSVLRGGKYAATSQVLLPSKDVMQGTDEHVVC |
| | | KVQHPNGNKEKNVPLPVIAELPPKVSVFVPPRDGFFGNPRKSKLICQATGFSPRQIQVSWLREGKQVGSGVT |
| | | TDQVQAEAKESGPTTYKVTSTLTIKESDWLSQSMFTCRVDHRGLTFQQNASSMCVPDQDTAIRVFAIPPSFA |
| | | SIFLTKSTKLTCLVTDLTTYDSVTISWTRQNGEAVKTHTNISESHPNATFSAVGEASICEDDWNSGERFTCT |
| | | VTHTDLPSPLKQTISRPKGVALHRPDVYLLPPAREQLNLRESATITCLVTGFSPADVFVQWMQRGQPLSPEK |
| | | YVTSAPMPEPQAPGRYFAHSILTVSEEEWNTGETYTCVVAHEALPNRVTERTVDKSTGKPTLYNVSLVMSDT |
| | | AGTCY |
| 106 | CoV2-14 Lambda Light | QAVVTQPASVSGSPGQSITISCTGTSSDIGAYNYISWYQQHPGKAPKLIIYEVSNRPSGISYRFSGSKSGNT |
| | Chain | ASLTISGLQAEDEANYYCSSYAGSISFGGGTKVTVLGQPKAAPSVTLFPPSSEELQANKATLVCLISDFYPG |
| | | AVTVAWKADSSPVKAGVETTTPSKQSNNKYAASSYLSLTPEQWKSHRSYSCQVTHEGSTVEKTVAPTECS |

What is claimed is:

1. A multimeric binding molecule comprising two to six bivalent binding units, wherein each binding unit comprises two IgM or IgA heavy chain constant regions or multimerizing fragments or variants thereof each associated with a binding domain,
wherein three to twelve of the binding domains are identical immunoglobulin antigen binding domains that specifically bind to the SARS-CoV-2 spike (S) protein receptor binding domain (RBD);
wherein each identical immunoglobulin antigen binding domain comprises a heavy chain variable region (VH) and a light chain variable region (VL) comprising six immunoglobulin complementarity determining regions HCDR1, HCDR2, HCDR3, LCDR1, LCDR2, and LCDR3, wherein the HCDR1, HCDR2, HCDR3, LCDR1, LCDR2, and LCDR3, comprise, respectively, the amino acid sequences
SEQ ID NOs: 39, 40, 41, 43, 44, and 45;
SEQ ID NOs: 15, 16, 17, 19, 20, and 21;
SEQ ID NOs: 23, 24, 25, 27, 28, and 29;
SEQ ID NOs: 31, 32, 33, 35, 36, and 37;
SEQ ID NOs: 47, 48, 49, 51, 52, and 53; or
SEQ ID NOs: 55, 56, 57, 59, 60, and 61;
wherein the CDR regions are defined according to Kabat; and wherein the multimeric binding molecule has greater antiviral potency against SARS-CoV-2 than a bivalent reference IgG antibody comprising two of the binding domains that specifically bind to the SARS-CoV-2 S protein RBD.

2. The multimeric binding molecule of claim 1, wherein the HCDR1, HCDR2, HCDR3, LCDR1, LCDR2, and LCDR3, comprise, respectively, the amino acid sequences SEQ ID NOs: 39, 40, 41, 43, 44, and 45, or SEQ ID NOs: 15, 16, 17, 19, 20, and 21.

3. The multimeric binding molecule of claim 2, wherein:
the VH and VL of the multimeric binding molecule comprise the amino acid sequences SEQ ID NO: 38 and 42, respectively, and the VH and VL of the bivalent reference IgG antibody comprise the amino acid sequences SEQ ID NOs: 38 and 42, respectively; or
the VH and VL of the multimeric binding molecule comprise the amino acid sequences SEQ ID NOs: 14 and 18, respectively, and the VH and VL of the bivalent reference IgG antibody comprise the amino acid sequences SEQ ID NOs: 14 and 18, respectively.

4. The multimeric binding molecule of claim 1, wherein the greater antiviral potency against SARS-CoV-2 comprises a) inhibition of binding of the SARS-CoV-2 spike protein to its receptor angiotensin-converting enzyme 2 (ACE2) at a lower 50% effective concentration (EC50) than the bivalent reference IgG antibody, b) inhibition of binding of the SARS-CoV-2 spike protein to ACE2 under conditions where the bivalent reference IgG antibody cannot inhibit binding, c) neutralization of SARS-CoV-2 infectivity at a lower EC50 than the bivalent reference IgG antibody, d) neutralization of SARS-CoV-2 infectivity under conditions where the bivalent reference IgG antibody cannot neutralize SARS-CoV-2 infectivity, e) protection against SARS-CoV-2 infection in a therapeutic animal model at a lower 50% effective dose (ED50) than the bivalent IgG antibody, f) protection against SARS-CoV-2 infection in the therapeutic animal model under conditions where the bivalent reference IgG antibody cannot protect, g) protection against SARS-CoV-2 infection in a prophylactic animal model at a lower ED50 than the bivalent IgG antibody, h) protection against SARS-CoV-2 infection in the prophylactic animal model under conditions where the bivalent reference IgG antibody cannot protect, or i) any combination thereof.

5. The multimeric binding molecule of claim 4, wherein the binding molecule can neutralize infectivity SARS-CoV-2 at a lower EC₅₀ than the bivalent reference IgG antibody or can neutralize infectivity of SARS-CoV-2 under conditions where the bivalent reference IgG antibody cannot neutralize.

6. The multimeric binding molecule of claim 5, wherein the EC₅₀ is at least ten-fold lower than the EC₅₀ of the bivalent IgG antibody.

7. The multimeric binding molecule of claim 4, wherein the multimeric binding molecule reduces, inhibits, or blocks the SARS-CoV-2 S protein from binding to ACE2 at a lower EC₅₀ than the bivalent reference IgG antibody or reduces, inhibits, or blocks the SARS-CoV-2 S protein from binding to ACE2 under conditions where the bivalent reference IgG antibody cannot reduce, inhibit, or block the SARS-CoV-2 S protein from binding to ACE2.

8. The multimeric binding molecule of claim 1, wherein the immunoglobulin antigen-binding domains are human immunoglobulin antigen-binding domains.

9. The multimeric binding molecule of claim 1, wherein each binding unit comprises two heavy chains comprising the VH and two light chains comprising the VL.

10. The multimeric binding molecule of claim 9, comprising five or six bivalent IgM or IgM-like binding units, wherein each binding unit comprises two IgM heavy chain constant regions or multimerizing fragments or variants thereof, each comprising an IgM Cμ4 domain and an IgM tailpiece domain.

11. The multimeric binding molecule of claim 10, wherein each IgM heavy chain constant region or multimerizing fragment or variant thereof further comprises a Cμ1 domain, a Cμ2 domain, a Cμ3 domain, or any combination thereof.

12. The multimeric binding molecule of claim 10, wherein the IgM heavy chain constant regions or multimerizing fragments or variants thereof are human IgM constant regions.

13. The multimeric binding molecule of claim 10, wherein the IgM heavy chain constant regions each comprise the amino acid sequence SEQ ID NO: 1, SEQ ID NO: 2, or a multimerizing fragment or variant thereof.

14. The multimeric binding molecule of claim 10, which is pentameric, and further comprises a J-chain or functional fragment or variant thereof.

15. The multimeric binding molecule of claim 14, wherein the heavy chains each comprise the amino acid sequence SEQ ID NO: 105, the light chains each comprise the amino acid sequence SEQ ID NO: 106, and the J-chain comprises the amino acid sequence SEQ ID NO: 7.

16. The multimeric binding molecule of claim 14, which can transport across vascular endothelial cells via J-chain binding to the polymeric Ig receptor (PIgR).

17. The multimeric binding molecule of claim 1, comprising two or four bivalent IgA or IgA-like binding units and a J chain or functional fragment or variant thereof, wherein each binding unit comprises two IgA heavy chain constant regions or multimerizing fragments or variants thereof, each comprising an IgA Cα3 domain and an IgA tailpiece domain.

18. The multimeric binding molecule of claim 17, wherein each IgA heavy chain constant region or multimerizing fragment or variant thereof further comprises a Cα1 domain, a Cα2 domain, an IgA hinge region, or any combination thereof.

19. The multimeric binding molecule of claim 17, wherein the IgA heavy chain constant regions or multimerizing fragments or variants thereof are human IgA constant regions.

20. The multimeric binding molecule of claim 17, wherein each binding unit comprises two IgA heavy chains each comprising a VH situated amino terminal to the IgA constant region or multimerizing fragment or variant thereof, and two immunoglobulin light chains each comprising a VL situated amino terminal to an immunoglobulin light chain constant region.

21. A composition comprising the multimeric binding molecule of claim 1.

22. A composition comprising two or more nonidentical multimeric binding molecules according claim 1, wherein the two or more multimeric binding molecules bind to different epitopes of the SARS-CoV-2 spike (S) protein receptor binding domain (RBD).

23. A polynucleotide comprising a nucleic acid sequence that encodes a polypeptide subunit of the binding molecule of claim 1.

24. A host cell comprising the polynucleotide of claim 23, wherein the host cell can express the multimeric binding molecule or a subunit thereof.

25. A method of producing a multimeric binding molecule, the method comprising culturing the host cell of claim 24 and recovering the multimeric binding molecule.

26. A method for treating or preventing coronavirus disease 2019 (COVID-19) disease in a subject, the method comprising administering to a subject in need of treatment an effective amount of the multimeric binding molecule of claim 1.

27. The method of claim 26, wherein the subject is human.

28. The method of claim 26, wherein the administering comprises intravenous, subcutaneous, intramuscular, intranasal, and/or inhalation administration.

29. The method of claim 28, wherein the administering comprises intranasal administration.

30. The method of claim 28, wherein the administering comprises inhalation administration.


SEQUENCE LISTING (106 sequences)

SEQ ID NO: 1 - protein, 453 aa, Artificial sequence [sequence omitted]

SEQ ID NO: 2 - protein, 453 aa, Artificial sequence
  Synthetic amino acid
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 3 - protein, 353 aa, Artificial sequence
  Synthetic amino acid
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 4 - protein, 340 aa, Artificial sequence
  Synthetic amino acid
ASPTSPKVFPLSLDSTPQDGNVVVACLVQGFFPQEPLSVTWSESGQNVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTYAVTSILRVAAEDWKKGETFSCMVGHEALPLAFTQKTIDRMAGKPTHINVSVVMAEADGTCY

SEQ ID NO: 5 - protein, 764 aa, Artificial sequence
  Synthetic amino acid
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRALVSTLVPLGLVLAVGAVAVGVARARHRKNVDRVSIRSYRTDISMSDFENSREFGANDNMGASSITQETSLGGKEEFVATTESTTETKEPKKAKRSSKEEAEMAYKDFLLQSSTVAAEAQDGPQEA

SEQ ID NO: 6 - protein, 159 aa, Artificial sequence
  Synthetic amino acid
MKNHLLFWGVLAVFIKAVHVKAQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 7 - protein, 137 aa, Artificial sequence
  Synthetic amino acid
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 8 - protein, 137 aa, Artificial sequence
  Synthetic amino acid
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 9 - protein, 5 aa, Artificial sequence
  Synthetic linker
GGGGS

SEQ ID NO: 10 - protein, 10 aa, Artificial sequence
  Synthetic linker
GGGGSGGGGS

SEQ ID NO: 11 - protein, 15 aa, Artificial sequence
  Synthetic linker
GGGGSGGGGSGGGGS

SEQ ID NO: 12 - protein, 20 aa, Artificial sequence
  Synthetic linker
GGGGSGGGGSGGGGSGGGGS

SEQ ID NO: 13 - protein, 25 aa, Artificial sequence
  Synthetic linker
GGGGSGGGGSGGGGSGGGGSGGGGS

SEQ ID NO: 14 - protein, 121 aa, Artificial sequence
  Synthetic antibody
QVELQESGPGLVKPSGTLSLTCAVSGGSISSNNWWTWVRQPPGKGLEWIGEIHHSGGTNYNPSLKSRVTMSVDKSKNQFSLNLYSVTAADTAVYYCTRDRAGGTYSGFDFWGQGTLVTVSS

SEQ ID NO: 15 - protein, 6 aa, Artificial sequence
  Synthetic antibody
SNNWWT

SEQ ID NO: 16 - protein, 16 aa, Artificial sequence
  Synthetic antibody
EIHHSGGTNYNPSLKS

SEQ ID NO: 17 - protein, 12 aa, Artificial sequence
  Synthetic antibody
DRAGGTYSGFDF

SEQ ID NO: 18 - protein, 110 aa, Artificial sequence
  Synthetic antibody
QAVVTQPASVSGSPGQSITISCTGTSSDVGGYNYVSWYQQHPGKAPKLMIYDVSNRPSGVSNRFSGSKSGNTASLTISGLQAEDEADYYCSSYTSSSTVVFGGGTKVTVL

SEQ ID NO: 19 - protein, 14 aa, Artificial sequence
  Synthetic antibody
TGTSSDVGGYNYVS

SEQ ID NO: 20 - protein, 7 aa, Artificial sequence
  Synthetic antibody
DVSNRPS

SEQ ID NO: 21 - protein, 10 aa, Artificial sequence
  Synthetic antibody
SSYTSSSTVV

SEQ ID NO: 22 - protein, 124 aa, Artificial sequence
  Synthetic antibody
QVQLHQWGAGLLKPSETLSRTCAVYGGSFSGYYWTWIRQAPGKGLEWIGEINHGGSTRYNPSLESRVSISVDTSKKQFSLELRSVTAADTAIYYCARGYDTNWYGDGYNWFDPWDRGTLVTVSS

SEQ ID NO: 23 - protein, 5 aa, Artificial sequence
  Synthetic antibody
GYYWT

SEQ ID NO: 24 - protein, 16 aa, Artificial sequence
  Synthetic antibody
EINHGGSTRYNPSLES

SEQ ID NO: 25 - protein, 16 aa, Artificial sequence
  Synthetic antibody
GYDTNWYGDGYNWFDP

SEQ ID NO: 26 - protein, 108 aa, Artificial sequence
  Synthetic antibody
QSALTQDPSVSVALGQTVRITCQGDSLRGSFASWYRQKPGQAPVLVIFGINNRPSGVPDRFSGSSSGNTASLTITGAQAEDEADYYCNSRESNSNRILFGGGTKVTVL

SEQ ID NO: 27 - protein, 11 aa, Artificial sequence
  Synthetic antibody
QGDSLRGSFAS

SEQ ID NO: 28 - protein, 7 aa, Artificial sequence
  Synthetic antibody
GINNRPS

SEQ ID NO: 29 - protein, 11 aa, Artificial sequence
  Synthetic antibody
NSRESNSNRIL

SEQ ID NO: 30 - protein, 123 aa, Artificial sequence
  Synthetic antibody
QVQLVESGGGVVQPGRSLRLSCVASGFTFSSYAMQWVRQAPGKGLEYVAVVSDDGNMKFYADSVKGRFTISRDNSRNTVSLQMNSLGVEDTAVYYCARENYFWSGSIGGLDYWGQGTLVSVSS

SEQ ID NO: 31 - protein, 5 aa, Artificial sequence
  Synthetic antibody
SYAMQ

SEQ ID NO: 32 - protein, 17 aa, Artificial sequence
  Synthetic antibody
VVSDDGNMKFYADSVKG

SEQ ID NO: 33 - protein, 14 aa, Artificial sequence
  Synthetic antibody
ENYFWSGSIGGLDY

SEQ ID NO: 34 - protein, 14 aa, Artificial sequence
  Synthetic antibody
ENYFWSGSIGGLDY

SEQ ID NO: 35 - protein, 11 aa, Artificial sequence
  Synthetic antibody
RASQSISSYLN

SEQ ID NO: 36 - protein, 7 aa, Artificial sequence
  Synthetic antibody
AASSLQS

SEQ ID NO: 37 - protein, 10 aa, Artificial sequence
  Synthetic antibody
QQSYSTPGYT

SEQ ID NO: 38 - protein, 128 aa, Artificial sequence
  Synthetic antibody
EVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREEQQLVHDYYYYGMDVWGQGTMVTVSS

SEQ ID NO: 39 - protein, 7 aa, Artificial sequence
  Synthetic antibody
SNSAAWN

SEQ ID NO: 40 - protein, 18 aa, Artificial sequence
  Synthetic antibody
RTYYRSKWYNDYAVSVKS

SEQ ID NO: 41 - protein, 16 aa, Artificial sequence
  Synthetic antibody
EEQQLVHDYYYYGMDV

SEQ ID NO: 42 - protein, 108 aa, Artificial sequence
  Synthetic antibody
QAVVTQPASVSGSPGQSITISCTGTSSDIGAYNYISWYQQHPGKAPKLIIYEVSNRPSGISYRFSGSKSGNTASLTISGLQAEDEANYYCSSYAGSISFGGGTKVTVL

SEQ ID NO: 43 - protein, 14 aa, Artificial sequence
  Synthetic antibody
TGTSSDIGAYNYIS

SEQ ID NO: 44 - protein, 7 aa, Artificial sequence
  Synthetic antibody
EVSNRPS

SEQ ID NO: 45 - protein, 8 aa, Artificial sequence
  Synthetic antibody
SSYAGSIS

SEQ ID NO: 46 - protein, 124 aa, Artificial sequence; identical to SEQ ID NO: 22
  Synthetic antibody

SEQ ID NO: 47 - protein, 5 aa, Artificial sequence
  Synthetic antibody
GYYWT

SEQ ID NO: 48 - protein, 16 aa, Artificial sequence
  Synthetic antibody
EINHGGSTRYNPSLES

SEQ ID NO: 49 - protein, 16 aa, Artificial sequence
  Synthetic antibody
GYDTNWYGDGYNWFDP

SEQ ID NO: 50 - protein, 110 aa, Artificial sequence
  Synthetic antibody
QAVVTQPPSVSGTPGQRVTISCSGSSSSIGSNTVHWYQHLPGTAPKLLMYNNNERPSGVPARFSGSKSGTSASLVITGLQADDEAEYYCQSYDTGLSGHVFGSGTELTVL

SEQ ID NO: 51 - protein, 13 aa, Artificial sequence
  Synthetic antibody
SGSSSSIGSNTVH

SEQ ID NO: 52 - protein, 7 aa, Artificial sequence
  Synthetic antibody
EVSNRPS

SEQ ID NO: 53 - protein, 11 aa, Artificial sequence
  Synthetic antibody
QSYDTGLSGHV

SEQ ID NO: 54 - protein, 114 aa, Artificial sequence
  Synthetic antibody
QVQLVESGAEVKKPGASVKVSCKASGYTFTGYYMHWVRQAPGQGLEWMGRINPNSGGTNYAQKFQGRVTMTRDTSISTAYMELSRLRSDDTAVYYCARGYFDYWGQGTLVTVSS

SEQ ID NO: 55 - protein, 5 aa, Artificial sequence
  Synthetic antibody
GYYMH

SEQ ID NO: 56 - protein, 17 aa, Artificial sequence
  Synthetic antibody
RINPNSGGTNYAQKFQG

SEQ ID NO: 57 - protein, 5 aa, Artificial sequence
  Synthetic antibody
GYFDY

SEQ ID NO: 58 - protein, 110 aa, Artificial sequence
  Synthetic antibody
QSALTQPPSASGTPGQRVTISCSGSSSNIGSNSVNWYQQVPGTAPKLLIYANDHRPSGVPDRFSGSKSGTSASLAISGLQSEDEADYYCAAWDNSLKGVVFGGGTKVTVL

SEQ ID NO: 59 - protein, 13 aa, Artificial sequence
  Synthetic antibody
SGSSSNIGSNSVN

SEQ ID NO: 60 - protein, 7 aa, Artificial sequence
  Synthetic antibody
ANDHRPS

SEQ ID NO: 61 - protein, 11 aa, Artificial sequence
  Synthetic antibody
AAWDNSLKGVV

SEQ ID NO: 62 - protein, 9 aa, Artificial sequence
  Synthetic antibody
GGSISSNNW

SEQ ID NO: 63 - protein, 7 aa, Artificial sequence
  Synthetic antibody
IHHSGGT

SEQ ID NO: 64 - protein, 14 aa, Artificial sequence
  Synthetic antibody
TRDRAGGTYSGFDF

SEQ ID NO: 65 - protein, 9 aa, Artificial sequence
  Synthetic antibody
SSDVGGYNY

SEQ ID NO: 66 - protein, 3 aa, Artificial sequence
  Synthetic antibody
DVS

SEQ ID NO: 67 - protein, 10 aa, Artificial sequence
  Synthetic antibody
SSYTSSSTVV

SEQ ID NO: 68 - protein, 8 aa, Artificial sequence
  Synthetic antibody
GGSFSGYY

SEQ ID NO: 69 - protein, 7 aa, Artificial sequence
  Synthetic antibody
INHGGST

SEQ ID NO: 70 - protein, 18 aa, Artificial sequence
  Synthetic antibody
ARGYDTNWYGDGYNWFDP

SEQ ID NO: 71 - protein, 6 aa, Artificial sequence
  Synthetic antibody
SLRGSF

SEQ ID NO: 72 - protein, 3 aa, Artificial sequence
  Synthetic antibody
GIN

SEQ ID NO: 73 - protein, 11 aa, Artificial sequence
  Synthetic antibody
NSRESNSNRIL

SEQ ID NO: 74 - protein, 8 aa, Artificial sequence
  Synthetic antibody
GFTFSSYA

SEQ ID NO: 75 - protein, 8 aa, Artificial sequence
  Synthetic antibody
VSDDGNMK

SEQ ID NO: 76 - protein, 16 aa, Artificial sequence
  Synthetic antibody
ARENYFWSGSIGGLDY

SEQ ID NO: 77 - protein, 6 aa, Artificial sequence
  Synthetic antibody
QSISSY

SEQ ID NO: 78 - protein, 3 aa, Artificial sequence
  Synthetic antibody
AAS

SEQ ID NO: 79 - protein, 10 aa, Artificial sequence
  Synthetic antibody
QQSYSTPGYT

SEQ ID NO: 80 - protein, 10 aa, Artificial sequence
  Synthetic antibody
GDSVSSNSAA

SEQ ID NO: 81 - protein, 9 aa, Artificial sequence
  Synthetic antibody
TYYRSKWYN

SEQ ID NO: 82 - protein, 18 aa, Artificial sequence
  Synthetic antibody
AREEQQLVHDYYYYGMDV

SEQ ID NO: 83 - protein, 9 aa, Artificial sequence
  Synthetic antibody
SSDIGAYNY

SEQ ID NO: 84 - protein, 3 aa, Artificial sequence
  Synthetic antibody
EVS

SEQ ID NO: 85 - protein, 8 aa, Artificial sequence
  Synthetic antibody
SSYAGSIS

SEQ ID NO: 86 - protein, 8 aa, Artificial sequence
  Synthetic antibody
GGSFSGYY

SEQ ID NO: 87 - protein, 7 aa, Artificial sequence
  Synthetic antibody
INHGGST

SEQ ID NO: 88 - protein, 18 aa, Artificial sequence
  Synthetic antibody
ARGYDTNWYGDGYNWFDP

SEQ ID NO: 89 - protein, 8 aa, Artificial sequence
  Synthetic antibody
SSSIGSNT

SEQ ID NO: 90 - protein, 3 aa, Artificial sequence
  Synthetic antibody
NNN

SEQ ID NO: 91 - protein, 11 aa, Artificial sequence
  Synthetic antibody
QSYDTGLSGHV

SEQ ID NO: 92 - protein, 8 aa, Artificial sequence
  Synthetic antibody
GYTFTGYY

SEQ ID NO: 93 - protein, 8 aa, Artificial sequence
  Synthetic antibody
INPNSGGT

SEQ ID NO: 94 - protein, 7 aa, Artificial sequence
  Synthetic antibody
ARGYFDY

SEQ ID NO: 95 - protein, 8 aa, Artificial sequence
  Synthetic antibody
SSNIGSNS

SEQ ID NO: 96 - protein, 3 aa, Artificial sequence
  Synthetic antibody
AND

SEQ ID NO: 97 - protein, 11 aa, Artificial sequence
  Synthetic antibody
AAWDNSLKGVV

SEQ ID NO: 98 - protein, 119 aa, Artificial sequence
  Synthetic antibody
QMQLVQSGTEVKKPGESLKISCKGSGYGFITYWIGWVRQMPGKGLEWMGIIYPGDSETRYSPSFQGQVTISADKSINTAYLQWSSLKASDTAIYYCAGGSGISTPMDVWGQGTTVTVSS

SEQ ID NO: 99 - protein, 113 aa, Artificial sequence
  Synthetic antibody
DIQLTQSPDSLAVSLGERATINCKSSQSVLYSSINKNYLAWYQQKPGQPPKLLIYWASTRESGVPDRFSGSGSGTDFTLTISSLQAEDVAVYYCQQYYSTPYTFGQGTKVEIK

SEQ ID NO: 100 - protein, 120 aa, Artificial sequence
  Synthetic antibody
EVQLVESGGGLVQPGGSLRLSCAASGFTFSDHYMDWVRQAPGKGLEWVGRTRNKANSYTTEYAASVKGRFTISRDDSKNSLYLQMNSLKTEDTAVYYCARGISPFYFDYWGQGTLVTVSS

SEQ ID NO: 101 - protein, 107 aa, Artificial sequence
  Synthetic antibody
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIK

SEQ ID NO: 102 - protein, 1273 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 103 - DNA, 20 bp, Artificial sequence
  Synthetic primer
ttacaaacattggccgcaaa

SEQ ID NO: 104 - DNA, 18 bp, Artificial sequence
  Synthetic primer
gcgcgacattccgaagaa

SEQ ID NO: 105 - protein, 581 aa, Artificial sequence
  Synthetic amino acid
EVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREEQQLVHDYYYYGMDVWGQGTMVTVSSGSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 106 - protein, 214 aa, Artificial sequence
  Synthetic amino acid
QAVVTQPASVSGSPGQSITISCTGTSSDIGAYNYISWYQQHPGKAPKLIIYEVSNRPSGISYRFSGSKSGNTASLTISGLQAEDEANYYCSSYAGSISFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS